(19) United States
(12) Patent Application Publication
(10) Pub. No.: US 2020/0179336 A1
(43) Pub. Date: Jun. 11, 2020

(54) METHODS OF TREATING NEUROLOGICAL AND PSYCHIATRIC DISORDERS

(71) Applicant: Sunovion Pharmaceuticals Inc., Marlborough, MA (US)

(72) Inventor: Seth Cabot HOPKINS, Northborough, MA (US)

(73) Assignee: Sunovion Pharmaceuticals Inc., Marlborough, MA (US)

(21) Appl. No.: 16/704,202

(22) Filed: Dec. 5, 2019

Related U.S. Application Data
(60) Provisional application No. 62/829,796, filed on Apr. 5, 2019.
(60) Provisional application No. 62/776,247, filed on Dec. 6, 2018.

(51) Int. Cl. A61K 31/381; A61P 25/18

(52) U.S. Cl. CPC A61K 31/381; A61P 25/18

(57) ABSTRACT

The present disclosure relates to methods of treating neurological or psychiatric diseases or disorders, such as schizophrenia. Compound 1, or a pharmaceutically acceptable salt thereof, is an antipsychotic agent with a non-D2 mechanism of action. Adverse events associated with antipsychotic agents that target the D2 dopamine receptor can be reduced by treating disorders with Compound 1, or a pharmaceutically acceptable salt thereof.

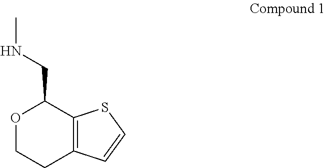

### CROSS REFERENCE TO RELATED APPLICATIONS

This application claims priority from U.S. provisional applications 62/776,247, filed Dec. 6, 2018, and 62/829,796, filed Apr. 5, 2019, the entire disclosures of which are hereby incorporated herein by reference.

### TECHNICAL FIELD

The present disclosure relates to methods of treating neurological and psychiatric diseases and disorders.

### BACKGROUND

The D2 dopamine receptor is a primary target for both typical and atypical antipsychotic agents. Wang et al. NATURE 555, 269-273 (2018). However, many drugs that target the D2 dopamine receptor cause serious or potentially life-threatening side effects. Wang et al. NATURE 555, 269-273 (2018). Despite decades of research on non-D2 mechanisms of action, developing non-D2 antipsychotic therapies that are both safe and effective has been challenging. Girgis et al., J. PSYCHIATRIC RES. (2018), https://doi.org/10.1016/j.jpsychires.2018.07.006. In particular, after performing a comprehensive review of literature relating to experimental treatments for schizophrenia, including 250 studies between 1970 to 2017 with glutamatergic, serotonergic, cholinergic, neuropeptidergic, hormone-based, dopaminergic, metabolic, vitamin/naturopathic, histaminergic, infection/inflammation-based, and otherwise miscellaneous mechanisms for treating schizophrenia, Girgis states, “Despite there being several promising [non-D2] targets, such as allosteric modulation of the NMDA and α7 nicotinic receptors, we cannot confidently state that any of the mechanistically novel experimental treatments covered in this review are definitely effective for the treatment of schizophrenia and ready for clinical use.” Accordingly, there is a need for therapeutic agents having efficacy in treating neurological and psychiatric diseases and disorders (e.g., schizophrenia) with lower incidence of adverse events.

As disclosed herein, Compound 1 has received Breakthrough Therapy Designation from the United States Food and Drug Administration (FDA) as a novel agent for the treatment of people with schizophrenia. Breakthrough Therapy Designation is intended to expedite the development and review of drugs for serious or life-threatening conditions when preliminary clinical evidence indicates that the drug may demonstrate substantial improvement over available therapy on one or more clinically significant endpoints. The FDA granted Breakthrough Therapy Designation for Compound 1 based on pivotal, Phase 2 data from clinical trials disclosed herein.

### SUMMARY

The present disclosure relates to methods of treating neurological and psychiatric diseases and disorders.

In some embodiments, provided is a method of treating a patient having a neurological or psychiatric disease or disorder, comprising orally administering to the patient Compound 1

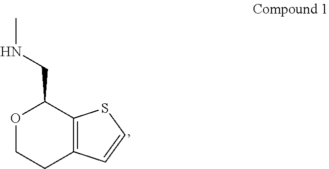

or a pharmaceutically acceptable salt thereof.

In some embodiments, provided is a method of treating a neurological or psychiatric disease or disorder in a patient, comprising administering to the patient a therapeutically effective amount of

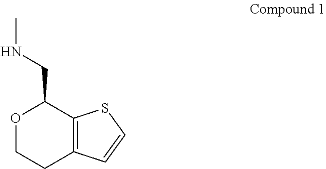

or a pharmaceutically acceptable salt thereof,
wherein the method minimizes adverse events in the patient. In some embodiments, the method minimizes adverse events associated with antipsychotic agents with affinity to dopamine D2 receptors.

In some embodiments, provided is a method of treating a neurological or psychiatric disease or disorder in a patient, comprising administering to the patient a therapeutically effective amount of Compound 1, or a pharmaceutically acceptable salt thereof, wherein the method is substantially devoid of adverse events. In some embodiments, a risk of adverse events in the patient is about the same as or similar to placebo.

In some embodiments, provided is a method of treating a neurological or psychiatric disease or disorder in a patient, wherein the method is substantially devoid of adverse events of an antipsychotic agent having affinity to dopamine D2 receptors, comprising administering to the patient a therapeutically effective amount of an antipsychotic agent with no direct affinity to dopamine D2 receptors selected from Compound 1, or a pharmaceutically acceptable salt thereof.

In some embodiments, provided is a method of minimizing adverse events in a patient in need of treatment for a neurological or psychiatric disease or disorder, the method comprising administering to the patient a therapeutically effective amount of an antipsychotic agent with no direct affinity to dopamine D2 receptors, wherein the antipsychotic agent is Compound 1, or a pharmaceutically acceptable salt thereof, and wherein the method minimizes adverse events associated with antipsychotic agents with affinity to dopamine D2 receptors.

In some embodiments, provided is a method of treating a neurological or psychiatric disease or disorder in a patient without subjecting the patient to a clinically significant risk of adverse events, the method comprising administering to the patient a therapeutically effective amount of Compound 1, or a pharmaceutically acceptable salt thereof, wherein the risk of adverse events are associated with antipsychotic agents with affinity to dopamine D2 receptors. In some embodiments, the disease or disorder is schizophrenia.

In some embodiments, provided is a method of administering an antipsychotic agent to a patient in need thereof without causing a clinically significant risk of adverse events, comprising administering to the patient a therapeutically effective amount of Compound 1, or a pharmaceutically acceptable salt thereof, wherein the patient does not experience a clinically significant adverse event.

In some embodiments, provided is a method of treating a patient having a neurological or psychiatric disease or disorder without causing a clinically significant risk of adverse events, comprising administering to the patient a therapeutically effective amount of Compound 1, or a pharmaceutically acceptable salt thereof. In some embodiments, the patient has schizophrenia.

In some embodiments, adverse events refers to one or more of the following: cardiovascular adverse events (e.g., atrial tachycardia, bradycardia, cardiovascular insufficiency, palpitations, postural tachycardia syndrome, increased blood pressure, hypertension, hypotension, hot flush, QT prolongation, orthostatic hypotension, or orthostatic tachycardia), extrapyramidal adverse events (e.g., akathisia, restlessness, joint stiffness, musculoskeletal stiffness, nuchal rigidity, postural tremor, or tremor), hyperprolactinemia, insomnia, anxiety, headaches, schizophrenia, somnolence, agitation, nausea, diarrhea, and dyspepsia.

In some embodiments, the method is efficacious for the treatment of the neurological or psychiatric disease or disorder in the patient. In some examples, the method results in improvement in one or more of Positive and Negative Symptom Scale (PANSS) total score, PANSS subscores (negative, positive, general psychopathology), Clinical Global Impressions-Severity (CGI-S) score, Brief Negative Symptom Scale (BNSS) total score, and Montgomery-Asberg Depression Rating Scale (MADRS) total score.

In some embodiments, provided is a method of treating a neurological or psychiatric disease or disorder in a patient, comprising administering to the patient a therapeutically effective amount of an antipsychotic agent with no direct affinity to dopamine D2 receptors, wherein the method is substantially devoid of adverse events in the patient, wherein the adverse events are associated with antipsychotic agents with affinity to dopamine D2.

In some embodiments, Compound 1, or a pharmaceutically acceptable salt thereof is Compound 1 hydrochloride of crystalline Form A.

### BRIEF DESCRIPTION OF THE DRAWINGS

FIG. 1 shows the MMRM analysis of change from baseline in PANSS total score for the study of Example 1.

FIG. 2 shows the MMRM analysis of change from baseline in PANSS positive subscale score for the study of Example 1.

FIG. 3 shows the MMRM analysis of change from baseline in PANSS negative subscale score for the study of Example 1.

FIG. 4 shows the MMRM analysis of change from baseline in PANSS general psychopathology subscale score for the study of Example 1.

FIG. 5 shows the MMRM analysis of change from baseline in CGI-S score for the study of Example 1.

FIG. 6 shows the MMRM analysis of change from baseline in BNSS total score for the study of Example 1.

FIG. 7 shows the MMRM analysis of change from baseline in MADRS total score for the study of Example 1.

FIG. 8 shows the median change from baseline to week 4 in prolactin levels for the study of Example 1.

FIG. 9 shows the observed PANSS total score during double-blind treatment (Example 1) and open-label extension study (Example 2).

FIG. 10 shows the observed PANSS positive subscore during double-blind treatment (Example 1) and open-label extension study (Example 2).

FIG. 11 shows the observed PANSS negative subscore during double-blind treatment (Example 1) and open-label extension study (Example 2).

FIG. 12 shows the observed PANSS general psychopathology subscore during double-blind treatment (Example 1) and open-label extension study (Example 2).

FIG. 13 shows the observed CGI-S score during double-blind treatment (Example 1) and open-label extension study (Example 2).

FIG. 14 shows the observed BNSS total score during double-blind treatment (Example 1) and open-label extension study (Example 2).

FIG. 15 shows the observed MADRS total score during double-blind treatment (Example 1) and open-label extension study (Example 2).

FIG. 16 shows the change from open-label baseline at week 26 in prolactin levels.

FIGS. 17A and 17B show the change from open-label baseline at week 26 in weight (FIG. 17A) and body mass index (BMI) (FIG. 17B).

FIGS. 18A to 18D show the change from open-label baseline at week 26 in lipids: total cholesterol (overall) (FIG. 18A), triglycerides (overall) (FIG. 18B), HDL (overall) (FIG. 18C), and LDL (overall) (FIG. 18D).

FIGS. 19A and 19B show the change from open-label baseline at week 26 in glycemic measures: glucose (overall) (FIG. 19A), and HbA1c (FIG. 19B).

FIGS. 20A and 20B show the time to all causes of discontinuation in the study of Example 2 (FIG. 20A) and comparative data for other drugs (FIG. 20B).

FIG. 21 and FIG. 22 present XRPD patterns for Compound 1 hydrochloride of crystalline Form A; FIG. 21 is the XRPD measured in transmission mode and FIG. 22 in reflection mode.

FIG. 23 is a DSC thermogram for Compound 1 hydrochloride of crystalline Form A.

### DETAILED DESCRIPTION

All published documents cited herein are hereby incorporated herein by reference in their entirety.

Unless defined otherwise, all technical and scientific terms used herein have the same meaning as commonly understood by one of ordinary skill in the art to which this disclosure belongs. Accordingly, the following terms are intended to have the following meanings:

As used herein, the singular forms “a”, “an” and “the” are intended to include the plural forms as well, unless the context clearly indicates otherwise.

Unless otherwise specified, the word “includes” (or any variation thereon, e.g., “include”, “including”, etc.) is intended to be open-ended. For example, “A includes 1, 2 and 3” means that A includes but is not limited to 1, 2 and 3.

As used herein, the terms “treatment,” “treat,” and “treating” refer to reversing, alleviating, delaying the onset of, or inhibiting the progress of a disease or disorder, or one or more symptoms thereof, including but not limited to therapeutic benefit. In some embodiments, treatment is administered after one or more symptoms have developed, for example, acute exacerbation of symptoms. In some embodiments, treatment may be administered in the absence of symptoms. For example, treatment may be administered to a subject prior to the onset of symptoms (e.g., in light of a history of symptoms and/or in light of genetic or other susceptibility factors). Treatment may also be continued after symptoms have resolved, for example to prevent or delay their recurrence.

Therapeutic benefit includes eradication and/or amelioration of the underlying disorder being treated; it also includes the eradication and/or amelioration of one or more of the symptoms associated with the underlying disorder such that an improvement is observed in the subject, notwithstanding that the subject may still be afflicted with the underlying disorder. In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life.)

As used herein, “administering” or “administration” of Compound 1, or a pharmaceutically acceptable salt thereof, encompasses the delivery to a subject of Compound 1, or a pharmaceutically acceptable salt thereof, or a prodrug or other pharmaceutically acceptable derivative thereof, using any suitable formulation or route of administration, e.g., as described herein.

As used herein, the term “therapeutically effective amount” or “effective amount” refers to an amount that is effective to elicit the desired biological or medical response, including the amount of a compound that, when administered to a subject for treating a disorder, is sufficient to effect such treatment of the disorder. The effective amount will vary depending on the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

As used herein, “delaying” development of a disorder mean to defer, hinder, slow, stabilize, and/or postpone development of the disorder. Delay can be of varying lengths of time, depending on the history of the disease and/or the individual being treated.

As used herein, “prevention” or “preventing” refers to a regimen that protects against the onset of the disorder such that the clinical symptoms of the disorder do not develop. Accordingly, “prevention” relates to administration of a therapy to a subject before signs of the diseases are detectable in the subject (for example, administration of a therapy in the absence of a detectable syndrome of the disorder). The subject may be an individual at risk of developing the disorder.

As used herein, an “at risk” individual is an individual who is at risk of developing a disorder to be treated. This may be shown, for example, by one or more risk factors, which are measurable parameters that correlate with development of a disorder and are known in the art.

As used herein, “subject” or “patient” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of Compound 1 include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Although pharmaceutically acceptable counter ions will be preferred for preparing pharmaceutical formulations, other anions are quite acceptable as synthetic intermediates. Thus X may be pharmaceutically undesirable anions, such as iodide, oxalate, trifluoromethanesulfonate and the like, when such salts are chemical intermediates.

As used herein, the term “pharmaceutically acceptable excipient” includes, without limitation, any binder, filler, adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, anti-caking agent, flavor, desiccants, plasticizers, disintegrants, lubricant, polymer matrix system, and polishing agents, which has been approved by the United States Food and Drug Administration as being acceptable for use in humans or domestic animals.

As used herein, a “clinically significant” risk of an adverse event refers to a risk that is greater than placebo by a statistically significant margin. When the risk of adverse events or a particular adverse event is less than, the same as, or about the same as placebo, the risk is not clinically significant.

As used herein, a “clinically meaningful” risk of an adverse event refers to a risk that is less than, but not necessarily by a statistically significant margin, the risk of the same adverse event in an antipsychotic agent with affinity to dopamine D2 receptors. When the risk of adverse events or a particular adverse event is less than an antipsychotic agent with affinity to dopamine D2 receptors, the risk is not clinically meaningful. In some embodiments, the risk of a clinically meaningful adverse event can be determined by one having ordinary skill in the art of treating and/or prescribing an antipsychotic agent to a patient in need. In some embodiments, the risk of a clinically meaningful adverse event can be determined comparative calculations across a patient population.

As used herein a method that is “substantially devoid” of adverse events refers to a method with an incidence of adverse events that is less than, the same as, or about the same as placebo.

As used herein “minimizing” adverse events refers to a statistically significant reduction in the incidence of adverse events in a patient population compared to the paradigmatic incidence of adverse events in a patient population treated with antipsychotic agents that have affinity to the D2 dopamine receptor. Such antipsychotic agents (e.g., as defined herein) that have affinity to the D2 dopamine receptor would have therapeutic affinity to the D2 dopamine receptor, such that one of skill in the art could propose direct targeting of the D2 dopamine receptor as a primary (either alone or in combination with another receptor) mechanism of action. The corresponding risk of adverse events in a single patient is reduced accordingly. In some embodiments, the incidence of an adverse event refers to the frequency or percentage of a specific adverse event over a patient population. In some embodiments, the incidence of an adverse event refers to the total number of adverse events experienced by an individual subject.

As used herein, “antipsychotic agents” are a class of medication specifically used to treat, prevent, or manage psychosis, for example in schizophrenia or bipolar disorder, and more broadly for treatment of various neurological and psychiatric disorders. First generation antipsychotic agents are known as “typical antipsychotics,” which include chlorpromazine, chlorprothixene, levomepromazine, mesoridazine, periciazine, promazine, thioridazine, loxapine, molindone, perphenazine, thiothixene, droperidol, flupentixol, fluphenazine, haloperidol, pimozide, prochlorperazine, thioproperazine, trifluoperazine and zuclopenthixol. Second generation antipsychotic agents are known as “atypical antipsychotics,” which include aripiprazole, asenapine maleate, clozapine, iloperidone, lurasidone, olanzapine, olanzapine/fluoxetine, paliperidone, quetiapine, risperidone, and ziprasidone. Both typical and atypical antipsychotics target and have affinity to D2 dopamine receptors.

“Adverse events associated with antipsychotic agents with affinity to dopamine D2 receptors” are understood by a person of ordinary skill in the art as adverse events that are paradigmatic of D2 antipsychotic therapy. In some embodiments, the adverse events associated with antipsychotic agents with affinity to dopamine D2 receptors is any one or more of class effects of antipsychotics agents. In some embodiments, the adverse events associated with antipsychotic agents with affinity to dopamine D2 receptors is any one or more of class effects of typical antipsychotics. In some embodiments, the adverse events associated with antipsychotic agents with affinity to dopamine D2 receptors is any one or more of class effects of atypical antipsychotics. In some embodiments, the adverse events associated with antipsychotic agents with affinity to dopamine D2 receptors are cardiovascular adverse events or extrapyramidal adverse events. In some embodiments, the adverse events associated with antipsychotic agents with affinity to dopamine D2 receptors include cardiovascular adverse events (e.g., atrial tachycardia, bradycardia, cardiovascular insufficiency, palpitations, postural tachycardia syndrome, increased blood pressure, hypertension, hypotension, hot flush, QT prolongation, orthostatic hypotension, or orthostatic tachycardia), extrapyramidal adverse events (e.g., akathisia, restlessness, joint stiffness, musculoskeletal stiffness, nuchal rigidity, postural tremor, or tremor), hyperprolactinemia, insomnia, anxiety, headaches, schizophrenia, somnolence, agitation, nausea, diarrhea, and dyspepsia.

The present disclosure describes various embodiments. A person of ordinary skill in the art reviewing the disclosure will readily recognize that various embodiments can be combined in any variation. For example, embodiments of the disclosure include treatment of various disorders, patient populations, administrations of dosage forms, at various dosages, minimization of various adverse events, and improvements in various efficacy measures, etc. Any combinations of various embodiments are within the scope of the disclosure.

Compound 1, as referred to herein for use in the methods of the present disclosure, has the following structure:

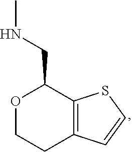

or a pharmaceutically acceptable salt thereof. Unless stated otherwise, or unless context requires otherwise, for purposes of this disclosure, the term “Compound 1” also includes pharmaceutically acceptable salts of:

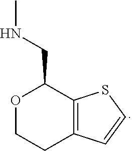

The chemical name for Compound 1 is (S)-(4,5-dihydro-7H-thieno[2,3-c]pyran-7-yl)-N-methylmethanamine (which may be abbreviated as “(S)-TPMA”), or a pharmaceutically acceptable salt thereof. One having ordinary skill in the art would appreciate the variety of nomenclature for compounds. Accordingly, Compound 1 may also be identified as (S)-1-(4,7-dihydro-5H-thieno[2,3-c]pyran-7-yl)-N-methylmethanamine, (S)-1-(5,7-dihydro-4H-thieno[2,3-c]pyran-7-yl)-N-methylmethanamine, or others, or a pharmaceutically acceptable salt thereof. For example, Compound 1, or a pharmaceutically acceptable salt thereof, has been identified as SEP-0363856 or SEP-856, and has received a Breakthrough Therapy Designation from the United States Food and Drug Administration (FDA) as a novel agent for the treatment of people with schizophrenia. Breakthrough Therapy Designation is intended to expedite the development and review of drugs for serious or life-threatening conditions when preliminary clinical evidence indicates that the drug may demonstrate substantial improvement over available therapy on one or more clinically significant endpoints. The FDA granted Breakthrough Therapy Designation for Compound 1, or a pharmaceutically acceptable salt thereof, based on pivotal, Phase 2 data from clinical trials disclosed herein. Compound 1, or a pharmaceutically acceptable salt thereof, is an antipsychotic agent with a non-direct-D2 mechanism of action, which shows broad efficacy in animal models of psychosis and depression. The molecular targets responsible for the antipsychotic and antidepressant efficacy of Compound 1, or a pharmaceutically acceptable salt thereof, are understood to be agonist activity at both trace amine associated receptor-1 (TAAR1) and 5HT_{1A} receptors. For example, as disclosed in Dedic et al. THE JOURNAL OF PHARMACOLOGY AND EXPERIMENTAL THERAPEUTICS 371, 1-14 (2019), Compound 1 was tested against several panels of known molecular targets (ion channels, G protein-coupled receptors (GPCRs), and enzymes, and, at 10 μM, Compound 1 showed >50% inhibition of specific binding at α_{2A}, α_{2B}, D₂, 5-HT_{1A}, 5-HT_{1B}, 5-HT_{1D}, 5-HT_{2A}, 5-HT_{2B}, 5-HT_{2C}, and 5-HT₇ receptors. Further receptor panel screening and follow-up functional testing showed that Compound 1 exhibited a range of activities at several receptors. Agonism at the human TAAR1 receptor (EC₅₀ of 0.14±0.062 μM, maximum efficacy (Eₘₐₓ)=101.3%±1.3%) and the 5-HT_{IA} receptor (EC₅₀=2.3 μM with values ranging from 0.1 to 3 μM, Eₘₐₓ=74.7%±19.6%). In D₂ receptor functional assays, Compound 1 exhibited weak partial agonism with EC₅₀ values of 10.44±4 μM (cAMP, Eₘₐₓ=23.9%±7.6%) and 8 μM (β-arrestin recruitment, Eₘₐₓ=27.1%). Without being bound to a particular mechanism of action, Compound 1 is theorized also to act as a pre-synaptic dopamine modulator.

Compound 1 can be used in the methods described herein as the free base or in the form of a pharmaceutically acceptable salt. In some embodiments, a hydrochloric acid (HCl) salt of Compound 1 is used in the methods described herein.

Compound 1, or a pharmaceutically acceptable salt thereof, can be obtained according to the production methods described in PCT Patent Publication No. WO2011/069063 (U.S. Pat. No. 8,710,245, issued Apr. 29, 2014) or PCT Patent Publication No. WO2019/161238, which are incorporated herein by reference in entirety and for all purposes, or a method analogous thereto.

Compound 1, or a pharmaceutically acceptable salt thereof, may be in amorphous or crystalline form. In some embodiments, a crystalline form of Compound 1, or a pharmaceutically acceptable salt thereof, is used in the methods described herein. In some embodiments, crystalline Form A of the HCl salt of Compound 1 is used in the methods described herein.

In some embodiments, crystalline Form A of the HCl salt of Compound 1 is characterized by a powder x-ray diffraction pattern comprising peaks, in terms of 2-theta, at 9.6±0.2°, 14.9±0.2°, 20.5±0.2°, and 25.1±0.2°, in some embodiments further comprising peaks at 20.2±0.2° and 20.8±0.2°, and in some embodiments further comprising peaks at 20.2±0.2° and 20.8±0.2° and a prominent peak at two or more of 17.9±0.2°, 24.8±0.2° and 27.1±0.2°. An example method of preparing crystalline Form A of the HCl salt of Compound 1 is provided in Example 2.

In some embodiments, Compound 1, or a pharmaceutically acceptable salt thereof, is substantially enantiomerically pure. In some examples, a composition comprising Compound 1, or a pharmaceutically acceptable salt thereof, comprises greater than or equal to about 90%, 95%, 97%, 99%, 99.5%, 99.7% or 99.9% of Compound 1, relative to the total amount of Compound 1 and its (R)-enantiomer in the composition. In some embodiments, a substantially enantiomerically pure crystalline Form A of the HCl salt of Compound 1 is used in the methods described herein.

Also provided herein are pharmaceutical compositions and dosage forms, comprising Compound 1, or a pharmaceutically acceptable salt thereof, and one or more pharmaceutically acceptable excipients. Compositions and dosage forms provided herein may further comprise one or more additional active ingredients. Compound 1, or a pharmaceutically acceptable salt thereof, may be administered as part of a pharmaceutical composition as described herein.

Selecting an appropriate pharmaceutical drug to treat a neurological or psychiatric disease or disorder involves finding a pharmaceutical drug which causes few or no adverse events in the patient. As disclosed herein, doctors wishing to avoid the trial and error approach to treating a neurological or psychiatric disease or disorder can select, from available antipsychotic agents, Compound 1 to treat the patient without a clinically significant risk of an adverse event. For example, for patients at risk for QT prolongation this can be important since QT prolongation is a serious adverse event which can lead to death. As disclosed herein, Compound 1, unlike some antipsychotics agents with affinity to dopamine D2 receptors, does not cause a clinically significant risk of QT prolongation, and may allow for safer dosing in patients who are at an elevated risk of QT prolongation. The risk of QT prolongation in patients who have not been previously treated with a neurological or psychiatric drug may be unknown. However, in some embodiments provided herein, Compound 1 can be administered without a clinically significant risk of QT prolongation. Other adverse events such as other cardiovascular events may take some time to manifest themselves, such as weight gain, a modification of the blood lipids or blood glucose levels. Over time, such events can cause cardiovascular issues in the patient. With the administration of Compound 1, the adverse events can be avoided or greatly reduced.

The present disclosure relates to methods of treating neurological and psychiatric diseases and disorders, such as schizophrenia.

In some embodiments, provided is a method of treating a patient having a neurological or psychiatric disease or disorder, comprising orally administering to the patient Compound 1

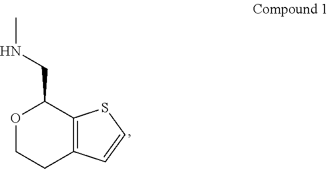

or a pharmaceutically acceptable salt thereof.

In some embodiments, provided is a method of treating a neurological or psychiatric disease or disorder in a patient, comprising administering to the patient a therapeutically effective amount of Compound 1, or a pharmaceutically acceptable salt thereof,

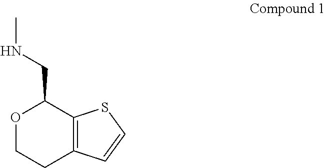

wherein the method minimizes adverse events in the patient. In some embodiments, the method minimizes adverse events associated with antipsychotic agents with affinity to dopamine D2 receptors.

In some embodiments, provided is a method of treating a neurological or psychiatric disease or disorder in a patient, comprising administering to the patient a therapeutically effective amount of Compound 1, or a pharmaceutically acceptable salt thereof, wherein the method is substantially devoid of adverse events. In some embodiments, the method produces a risk of adverse events in the patient that is about the same as or similar to placebo.

In some embodiments, provided is a method of treating a neurological or psychiatric disease or disorder in a patient, wherein the method is substantially devoid of adverse events of an antipsychotic agent having affinity to dopamine D2 receptors, comprising administering to the patient a therapeutically effective amount of an antipsychotic agent with no direct affinity to dopamine D2 receptors selected from Compound 1, or a pharmaceutically acceptable salt thereof.

In some embodiments, provided is a method of minimizing adverse events in a patient in need of treatment for a neurological or psychiatric disease or disorder, the method comprising administering to the patient a therapeutically effective amount of an antipsychotic agent with no direct affinity to dopamine D2 receptors, wherein the antipsychotic agent is Compound 1, or a pharmaceutically acceptable salt thereof, and wherein the method minimizes adverse events associated with antipsychotic agents with affinity to dopamine D2 receptors. In some embodiments, the method has reduced incidence of such adverse events compared to treatment with antipsychotic agents with affinity to dopamine D2 receptors.

In some embodiments, provided is a method of treating a neurological or psychiatric disease or disorder in a patient without subjecting the patient to a clinically significant risk of adverse events, the method comprising administering to the patient a therapeutically effective amount of Compound 1, or a pharmaceutically acceptable salt thereof, wherein the risk of adverse events are associated with antipsychotic agents with affinity to dopamine D2 receptors.

In some embodiments, provided is a method of administering an antipsychotic agent to a patient in need thereof without causing a clinically significant risk of adverse events, comprising administering to the patient a therapeutically effective amount of Compound 1, or a pharmaceutically acceptable salt thereof, wherein the patient does not experience a clinically significant adverse event. In some embodiments, the method treats a neurological or psychiatric disease or disorder (e.g., schizophrenia) in the patient.

In some embodiments, provided is a method of treating a neurological or psychiatric disease or disorder in a patient, comprising administering to the patient a therapeutically effective amount of an antipsychotic agent with no direct affinity to dopamine D2 receptors, wherein the method is substantially devoid of adverse events in the patient, wherein the adverse events are associated with antipsychotic agents with affinity to dopamine D2.

In some embodiments, provided is the use of Compound 1, or a pharmaceutically acceptable salt thereof, in the treatment of a neurological or psychiatric disease or disorder as generally described herein, e.g., with minimized adverse events. Also provided is Compound 1, or a pharmaceutically acceptable salt thereof, for use in the treatment of a neurological or psychiatric disease or disorder as generally described herein, e.g., with minimized adverse events. Also provided is use of Compound 1, or a pharmaceutically acceptable salt thereof, in the manufacture of a medicament for the treatment of a neurological or psychiatric disease or disorder as generally described herein, e.g., with minimized adverse events.

In some embodiments, Compound 1, or a pharmaceutically acceptable salt thereof, is administered daily for a 29-day treatment period. In some embodiments, Compound 1, or a pharmaceutically acceptable salt thereof, is administered daily for a 26-week or 30-week treatment period.

In some embodiments, adverse events refers to any one or more of the following: cardiovascular adverse events (atrial tachycardia, bradycardia, cardiovascular insufficiency, palpitations, postural tachycardia syndrome, increased blood pressure, hypertension, hypotension, hot flush, QT prolongation, orthostatic hypotension, orthostatic tachycardia), extrapyramidal adverse events (akathisia, restlessness, joint stiffness, musculoskeletal stiffness, nuchal rigidity, postural tremor, tremor), hyperprolactinemia, insomnia, anxiety, headaches, schizophrenia, somnolence, agitation, nausea, diarrhea, and dyspepsia.

In some embodiments, the methods of the present disclosure are efficacious for the treatment of the neurological or psychiatric disease or disorder in the patient. In some examples, the method results in improvement in one or more of Positive and Negative Symptom Scale (PANSS) total score, PANSS subscores (negative, positive, general psychopathology), Clinical Global Impressions-Severity (CGI-S) score, Brief Negative Symptom Scale (BNSS) total score, and Montgomery-Asberg Depression Rating Scale (MADRS) total score.

In some embodiments, the method results in one or more of:
a reduction from baseline in PANSS total score, e.g., a reduction from baseline in PANSS total score of at least 1, 2, 3, 4, 5, 7, 10, 15, or 17 (e.g., at least 17.2) or a reduction from baseline in PANSS total score of at least 1%, 2%, 3%, 4%, 5%, 7%, 10%, 15%, or 20% or a PANSS total score effect size of at least 0.1, 0.2, 0.3, or 0.4 (e.g., at least 0.45) or a PANSS responder rate with statistically significant improvement over placebo (e.g., at least 1%, 2%, 3%, 4%, 5%, 7%, 10%, 15% or 20% better responder rate than placebo);
a reduction from baseline in PANSS negative subscale score, e.g., a reduction from baseline in PANSS negative subscale score of at least 0.25. 0.5, 0.75, 1, 1.5, 2, 2.5 or 3 (e.g., at least 3.1) or a reduction from baseline in PANSS negative subscale score of at least 1%, 2%, 3%, 4%, 5%, 7%, 10%, 15%, or 20% or a PANSS negative subscale score effect size of at least 0.1, 0.2, 0.3 or 0.35 (e.g., at least 0.37);
a reduction from baseline in PANSS positive subscale score, e.g., a reduction from baseline in PANSS positive subscale score of at least 1, 2, 3, 4, or 5 (e.g., at least 5.5) or a reduction from baseline in PANSS positive subscale score of at least 1%, 2%, 3%, 4%, 5%, 7%, 10%, 15%, or 20% or a PANSS positive subscale score effect size of at least 0.1, 0.2 or 0.3 (e.g., at least 0.32);
a reduction from baseline in PANSS general psychopathology subscale score, e.g., a reduction from baseline in PANSS general psychopathology subscale score of at least 1, 2, 3, 4, 5, 7 or 9 (e.g., at least 9) or a reduction from baseline in PANSS general psychopathology subscale score of at least 1%, 2%, 3%, 4%, 5%, 7%, 10%, 15%, or 20% or a PANSS general psychopathology subscale score effect size of at least 0.1, 0.2, 0.3, 0.4 or 0.5 (e.g., at least 0.51);
a reduction from baseline in CGI-S score, e.g., a reduction from baseline in CGI-S score of at least 0.2, 0.4, 0.6, 0.8 or 1 (e.g., at least 1) or a reduction from baseline in CGI-S score of at least 1%, 2%, 3%, 4%, 5%, 7%, 10%, 15%, or 20% or a CGI-S score effect size of at least 0.1, 0.2, 0.3, 0.4 or 0.5 (e.g., at least 0.52);
a reduction from baseline in BNSS total score, e.g., a reduction from baseline in BNSS total score of at least 1, 2, 3, 4, 5, 6 or 7 (e.g., at least 7.1) or a reduction from baseline in BNSS total score of at least 1%, 2%, 3%, 4%, 5%, 7%, 10%, 15%, or 20% or a BNSS total score effect size of at least 0.1, 0.2, 0.3, 0.4, or 0.45 (e.g., at least 0.48); and
a reduction from baseline in MADRS total score, e.g., a reduction from baseline in MADRS total score of at least 0.5, 1, 1.5, 2, 2.5 or 3 (e.g., at least 3.3) or a reduction from baseline in MADRS total score of at least 1%, 2%, 3%, 4%, 5%, 7%, 10%, 15%, or 20% or a MADRS total score effect size of at least 0.1, 0.2, or 0.3 (e.g., at least 0.32).

In some embodiments, the reduction in PANSS (total or subscore), CGI-S, BNSS, or MADRS score is measured after a 29-day treatment period.

In some embodiments, the reduction in score is measured after a 30-week treatment period. In some embodiments, the methods of the present disclosure result in (i) a reduction from baseline in PANSS total score of at least about 30 after 30 weeks of treatment; (ii) a reduction from baseline in PANSS positive subscale score of at least about 10 after 30 weeks of treatment; (iii) a reduction from baseline in PANSS negative subscale score of at least about 5 after 30 weeks of treatment; (iv) a reduction from baseline in PANSS general psychopathology subscale score of at least about 15 after 30 weeks of treatment; (v) a reduction from baseline in CGI-S score of at least about 1.5 after 30 weeks of treatment, (vi) a reduction from baseline in BNSS total score of at least about 10 after 30 weeks of treatment; and/or (vii) a reduction from baseline in MADRS total score of at least about 5 after 30 weeks of treatment.

In some embodiments, the methods of the present disclosure result in a reduced number of adverse events leading to discontinuation during a treatment period, e.g., 29 days, 26 weeks, or 30 weeks. For example, in some embodiments, the method results in less than 50%, less than 40% or less than 35% of patients discontinuing treatment due to adverse events over a 26-week or 30-week treatment period.

In some embodiments, the neurological or psychiatric disease or disorder is schizophrenia. In some embodiments, the patient has acute exacerbation of schizophrenia. In some embodiments, treating schizophrenia comprises ameliorating a symptom of schizophrenia. In some embodiments, treating schizophrenia comprises treating negative symptoms of schizophrenia.

In some embodiments, the neurological or psychiatric disease or disorder is Parkinson's disease psychosis.

In some embodiments, the neurological or psychiatric disease or disorder is schizophrenia spectrum disorder, schizophrenia negative symptoms, attenuated psychosis syndrome, prodromal schizophrenia, delusional disorder, psychosis, psychotic disorder, delirium, Tourette's syndrome, post-traumatic stress disorder, behavior disorder, affective disorder, depression, bipolar disorder, major depressive disorder, dysthymia, manic disorder, seasonal affective disorder, obsessive-compulsive disorder, narcolepsy, REM behavior disorder, substance abuse or dependency, Lesch-Nyhan disease, Wilson's disease, autism, Alzheimer's disease agitation and psychosis, or Huntington's chorea.

In some embodiments, the neurological or psychiatric disease or disorder is selected from schizophrenia, attenuated psychosis syndrome, prodromal schizophrenia, schizoid personality disorder, and schizotypal personality disorder.

In some embodiments, the neurological or psychiatric disease or disorder is Alzheimer's disease agitation and psychosis. In some embodiments, the patient has dementia. In some embodiments, the neurological or psychiatric disease or disorder is dementia-related psychosis.

In some embodiments, the psychosis is selected from organic psychosis, drug-induced psychosis, Parkinson's disease psychosis, and excitative psychosis.

In some embodiments, the neurological or psychiatric disease or disorder is a bipolar disorder selected from bipolar disorder and bipolar depression.

In some embodiments, the patient fails to respond adequately to antipsychotic agents which are at least one typical antipsychotic agent or at least one atypical antipsychotic agent. In some embodiments, the patient fails to respond adequately to antipsychotic agents wherein the antipsychotic agents are typical antipsychotics or atypical antipsychotics. In some embodiments, the patient fails to respond adequately to antipsychotic agents wherein the antipsychotic agents are typical antipsychotics (e.g., chlorpromazine, chlorprothixene, levomepromazine, mesoridazine, periciazine, promazine, thioridazine, loxapine, molindone, perphenazine, thiothixene, droperidol, flupentixol, fluphenazine, haloperidol, pimozide, prochlorperazine, thioproperazine, trifluoperazine, zuclopenthixol) or atypical antipsychotics (e.g., aripiprazole, asenapine maleate, clozapine, iloperidone, lurasidone, olanzapine, olanzapine/fluoxetine, paliperidone, quetiapine, risperidone, ziprasidone)

In some embodiments, the patient is geriatric.

In some embodiments, treating a neurological or psychiatric disease or disorder comprises ameliorating a symptom of the neurological or psychiatric disease or disorder.

In some embodiments, the patient is characterized by one or more of:
the patient is an adult;
the patient has been diagnosed with schizophrenia for at least 6 months;
the patient has been experiencing acute exacerbation of psychotic symptoms for at least 2 months;
the patient has had no more than 2 prior hospitalizations for treatment of acute exacerbation of schizophrenia;
the patient has a baseline PANSS total score of at least 80;
the patient has a baseline PANSS score of at least 4 on two or more of: delusions, conceptual disorganization, hallucinatory behavior, and unusual thought content; and
the patient has a baseline CGI-S score of at least 4.

In some embodiments, adverse events refers to one or more of the following:
cardiovascular adverse events (e.g., atrial tachycardia, bradycardia, cardiovascular insufficiency, palpitations, postural tachycardia syndrome, increased blood pressure, hypertension, hypotension, hot flush, QT prolongation, orthostatic hypotension, or orthostatic tachycardia), extrapyramidal adverse events (e.g., akathisia, restlessness, joint stiffness, musculoskeletal stiffness, nuchal rigidity, postural tremor, or tremor), hyperprolactinemia, insomnia, anxiety, headaches, schizophrenia, somnolence, agitation, nausea, diarrhea, and dyspepsia.

The D2 dopamine receptor is the primary target for both typical and atypical antipsychotic agents. Wang et al. NATURE 555, 269-273 (2018). Unfortunately, many drugs that target the D2 dopamine receptor cause serious and potentially life-threatening side effects due to promiscuous activities against related receptors. Wang et al. NATURE 555, 269-273 (2018). Currently available antipsychotic agents that have affinity to the D2 dopamine receptor include typical antipsychotics, such as chlorpromazine, chlorprothixene, levomepromazine, mesoridazine, periciazine, promazine, thioridazine, loxapine, molindone, perphenazine, thiothixene, droperidol, flupentixol, fluphenazine, haloperidol, pimozide, prochlorperazine, thioproperazine, trifluoperazine and zuclopenthixol and atypical antipsychotics, such as aripiprazole, asenapine maleate, clozapine, iloperidone, lurasidone, olanzapine, olanzapine/fluoxetine, paliperidone, quetiapine, risperidone, and ziprasidone. Adverse events associated with typical and atypical antipsychotics include cardiovascular adverse events (e.g., atrial tachycardia, bradycardia, cardiovascular insufficiency, palpitations, postural tachycardia syndrome, increased blood pressure, hypertension, hypotension, hot flush, QT prolongation, orthostatic hypotension, or orthostatic tachycardia), extrapyramidal adverse events (e.g., akathisia, restlessness, joint stiffness, musculoskeletal stiffness, nuchal rigidity, postural tremor, or tremor), and hyperprolactinemia, insomnia, anxiety, headaches, schizophrenia, somnolence, agitation, nausea, diarrhea, and dyspepsia.

In some embodiments, the adverse events associated with antipsychotics are any one or more of class-effect adverse events as defined by EBGM rankings using FAERS. In some embodiments, the adverse events associated with antipsychotics are any one or more of: Hyperprolactinaemia, Blood prolactin abnormal, Blood prolactin increased, Galactorrhoea, Cogwheel rigidity, Obesity, Metabolic syndrome, Akathisia, Oromandibular dystonia, Parkinsonism, Drooling, Oculogyric crisis, Obsessive-compulsive disorder, Muscle rigidity, Type 2 diabetes mellitus, Diabetes mellitus, Overweight, Parkinsonian gait, Tongue spasm, Tardive dyskinesia, Bradykinesia, Tic, Psychomotor retardation, Extrapyramidal disorder, Enuresis, Glucose tolerance impaired, Salivary hypersecretion, Dystonia, Glycosuria, Restlessness, Torticollis, Impaired fasting glucose, Dermatillomania, Body mass index increased, Hyperkinesia, Hepatitis viral, Dyskinesia, Blood triglycerides increased, Electrocardiogram QT prolonged, Dyssomnia, Orthostatic hypertension, Bruxism, Increased appetite, Excessive eye blinking, Pancreatitis chronic, Weight increased, Dyslipidaemia, Restless legs syndrome, Tongue biting, or Nuchal rigidity.

In some embodiments, Compound 1 does not cause a clinically significant increase in the risk of an adverse event of any one or more of Hyperprolactinaemia, Blood prolactin abnormal, Blood prolactin increased, Galactorrhoea, Cogwheel rigidity, Obesity, Metabolic syndrome, Akathisia, Oromandibular dystonia, Parkinsonism, Drooling, Oculogyric crisis, Obsessive-compulsive disorder, Muscle rigidity, Type 2 diabetes mellitus, Diabetes mellitus, Overweight, Parkinsonian gait, Tongue spasm, Tardive dyskinesia, Bradykinesia, Tic, Psychomotor retardation, Extrapyramidal disorder, Enuresis, Glucose tolerance impaired, Salivary hypersecretion, Dystonia, Glycosuria, Restlessness, Torticollis, Impaired fasting glucose, Dermatillomania, Body mass index increased, Hyperkinesia, Hepatitis viral, Dyskinesia, Blood triglycerides increased, Electrocardiogram QT prolonged, Dyssomnia, Orthostatic hypertension, Bruxism, Increased appetite, Excessive eye blinking, Pancreatitis chronic, Weight increased, Dyslipidaemia, Restless legs syndrome, Tongue biting, or Nuchal rigidity.

Compound 1, or a pharmaceutically acceptable salt thereof, does not have direct affinity for the D2 dopamine receptor. As described herein (e.g., in the Example below), Compound 1, or a pharmaceutically acceptable salt thereof, when administered to patients, did not cause the high incidence of adverse events and serious adverse events associated with typical or atypical antipsychotic agents that target the D2 dopamine receptor. Surprisingly, as described in the Examples herein (e.g., Example 1, Example 2), Compound 1 had robust efficacy, yet with an adverse event profile similar to placebo. In particular, the incidence of cardiovascular adverse events (including QT prolongation, orthostatic hypotension, orthostatic tachycardia), extrapyramidal adverse events, hyperprolactinemia, insomnia, anxiety, and headaches, experienced by patients was not clinically significant (i.e., less than, the same as, or about the same as or similar to placebo).

In some embodiments, the methods of the present disclosure minimize cardiovascular adverse events. In some embodiments, the method is substantially devoid of cardiovascular adverse events. In some embodiments, the risk of cardiovascular adverse events in the patient is about the same as or similar to placebo. In some embodiments, the method results in a cardiovascular event in less than or equal to 5% of patients. In some embodiments, the method results in a cardiovascular adverse event in less than or equal to 4.2% of patients. In some embodiments, the method results in a cardiovascular adverse event in less than or equal to 5% (e.g., less than or equal to 4.2%) of patients during a 29-day treatment period. In some embodiments, the method results in a cardiovascular event in less than or equal to 6% (e.g., less than or equal to 5.8%) of patients during a 26-week treatment period. In some embodiments, the method results in a cardiovascular adverse event in a percentage of patients that is no more than 1% greater than placebo.

In some embodiments, the patient has an elevated risk of a cardiovascular adverse event from administration of an antipsychotic agent that has direct affinity to dopamine D2 receptors. In some embodiments, the patient has a history of cardiovascular disease. In some embodiments, the patient has a history of a cardiovascular adverse event from a prior antipsychotic therapy. In some embodiments, the patient is susceptible to a cardiovascular adverse event from an antipsychotic agent that has direct affinity to dopamine D2 receptors.

In some embodiments, the patient is not actively monitored for cardiovascular adverse events during a treatment period. In some embodiments, the patient is not monitored by electrocardiography monitoring during a treatment period. In some embodiments, the patient is not warned about cardiovascular adverse events. In some embodiments, the patient is not concurrently treated for cardiovascular adverse events.

In some embodiments, a cardiovascular adverse event is characterized as atrial tachycardia, bradycardia, cardiovascular insufficiency, palpitations, postural tachycardia syndrome, increased blood pressure, hypertension, hypotension, or hot flush. In some embodiments, a cardiovascular adverse event is characterized as atrial tachycardia, bradycardia, cardiovascular insufficiency, palpitations, postural tachycardia syndrome, increased blood pressure, hypertension, hypotension, hot flush, QT prolongation, orthostatic hypotension, or orthostatic tachycardia.

In some embodiments, the methods of the present disclosure minimize extrapyramidal adverse events. In some embodiments, the method is substantially devoid of extrapyramidal adverse events. In some embodiments, the risk of extrapyramidal adverse events in the patient is about the same as or similar to placebo. In some embodiments, the method results in an extrapyramidal adverse event in less than or equal to 5% of patients. In some embodiments, the method results in an extrapyramidal adverse event in less than or equal to 3.3% of patients. In some embodiments, the method results in an extrapyramidal adverse event in less than or equal to 5% of patients during a 29-day treatment period. In some embodiments, the method results in an extrapyramidal adverse even in less than or equal to 5% (e.g., less than or equal to 3.2%) of patients during a 26-week treatment period. In some embodiments, the method results in an extrapyramidal adverse event in a percentage of patients that is no more than placebo.

In some embodiments, the patient has an elevated risk of an extrapyramidal adverse event from administration of an antipsychotic agent that has direct affinity to dopamine D2 receptors. In some embodiments, the patient has a history of an extrapyramidal adverse event from a prior antipsychotic therapy. In some embodiments, the patient is susceptible to an extrapyramidal adverse event from an antipsychotic agent that has direct affinity to dopamine D2 receptors.

In some embodiments, the patient is not warned about extrapyramidal adverse events.

In some embodiments, an extrapyramidal adverse event is characterized as akathisia, restlessness, joint stiffness, musculoskeletal stiffness, nuchal rigidity, postural tremor, or tremor.

In some embodiments, methods of the present disclosure minimize QT prolongation. In some embodiments, the method is substantially devoid of QT prolongation. In some embodiments, the risk of QT prolongation in the patient is about the same as or similar to placebo. In some embodiments, the method results in QT prolongation in less than or equal to 5% of patients. In some embodiments, the method results in QT prolongation in less than or equal to 1% of patients. In some embodiments, the method is substantially devoid of QT prolongation during a 29-day treatment period. In some embodiments, the method results in QT prolongation in a percentage of patients that is no more than placebo.

In some embodiments, the patient has an elevated risk of QT prolongation from administration of an antipsychotic agent. In some embodiments, the patient has a history of QT prolongation from a prior antipsychotic therapy. In some embodiments, the patient is susceptible to QT prolongation from an antipsychotic agent that has direct affinity to dopamine D2 receptors. In some embodiments, the patient has hypokalemia, Hepatitis C, HIV, T-wave abnormalities on electrocardiogram, is female, is geriatric, or is taking a second active agent known to increase risk of QT prolongation.

In some embodiments, the patient is not actively monitored for QT prolongation. In some embodiments, the patient is not warned about QT prolongation. In some embodiments, the patient is not concurrently treated for QT prolongation.

In some embodiments, QT prolongation is characterized as one or both of:
a QTcF interval in the patient of greater than 450 msec at any time point not present at baseline; and
an increase in QTcF interval from baseline of greater than or equal to 30 msec for at least one post-baseline measurement.

In some embodiments, QT prolongation is characterized as one or both of:
a QTcF interval in the patient of greater than 450 msec at any time point not present at baseline if the patient is a male or greater than 470 msec at any time point not present at baseline if the patient is a female; and
an increase in QTcF interval from baseline of greater than or equal to 30 msec for at least one post-baseline measurement.

In some embodiments, QT prolongation is characterized as one or both of:
a QTcF interval in the patient of greater than 450 msec at any time point not present at baseline if the patient is a male or greater than 470 msec at any time point not present at baseline if the patient is a female; and
an increase in QTcF interval from baseline of greater than or equal to 60 msec for at least one post-baseline measurement.

Prolongation of the QTc interval of the electrocardiogram (ECG) may be associated with the development of torsade de pointes, a ventricular arrhythmia that can cause syncope and may progress to ventricular fibrillation and sudden death. The average QTc interval in healthy adults is approximately 400 msec. A QTc interval of 500 msec or greater is considered to be a substantial risk factor for torsade de pointes.

In some embodiments, the methods of the present disclosure minimize hyperprolactinemia. In some embodiments, the method is substantially devoid of hyperprolactinemia. In some embodiments, the risk of hyperprolactinemia in the patient is about the same as or similar to placebo. In some embodiments, the method does not have a clinically significant risk of hyperprolactinemia. In some embodiments, the method is substantially devoid of hyperprolactinemia during a 29-day treatment period. In some embodiments, the method is substantially devoid of hyperprolactinemia during a 26-week treatment period. In some embodiments, the method results in hyperprolactinemia in a percentage of patients that is no more than placebo.

In some embodiments, the patient has an elevated risk of hyperprolactinemia from administration of an antipsychotic agent that has direct affinity to dopamine D2 receptors. In some embodiments, the patient has a history of hyperprolactinemia from a prior antipsychotic therapy. In some embodiments, the patient is susceptible to hyperprolactinemia from an antipsychotic agent that has direct affinity to dopamine D2 receptors.

In some embodiments, the patient is not actively monitored for hyperprolactinemia. In some embodiments, the patient is not warned about hyperprolactinemia. In some embodiments, the patient is not concurrently treated for hyperprolactinemia.

Hyperprolactinemia refers to significantly elevated levels of prolactin and is known to occur during administration of certain antipsychotic agents.

In some embodiments, the metabolic effects of the method are the same or similar to placebo, e.g., total cholesterol, HDL cholesterol, LDL cholesterol, triglycerides, and/or glucose levels in the patient are the same or similar to placebo. In some embodiments, the method does not result in clinically significant weight gain.

In some embodiments, the methods of the present disclosure minimize orthostatic hypotension. In some embodiments, the method is substantially devoid of orthostatic hypotension. In some embodiments, the risk of orthostatic hypotension in the patient is about the same as or similar to placebo. In some embodiments, the method results in orthostatic hypotension in less than or equal to 5% of patients. In some embodiments, the method results in orthostatic hypotension in less than or equal to 4.2% of patients. In some embodiments, the method results in orthostatic hypotension in less than or equal to 5% of patients during a 29-day treatment period. In some embodiments, the method results in orthostatic hypotension in a percentage of patients that is no more than placebo.

In some embodiments, the patient has an elevated risk of orthostatic hypotension from administration of an antipsychotic agent. In some embodiments, the patient has a history of orthostatic hypotension from a prior antipsychotic therapy. In some embodiments, the patient is susceptible to orthostatic hypotension from an antipsychotic agent that has direct affinity to dopamine D2 receptors.

In some embodiments, the patient is not actively monitored for orthostatic hypotension. In some embodiments, the patient is not warned about orthostatic hypotension. In some embodiments, the patient is not concurrently treated for orthostatic hypotension.

In some embodiments, the methods of the present disclosure minimize orthostatic tachycardia. In some embodiments, the method is substantially devoid of orthostatic tachycardia. In some embodiments, the risk of orthostatic tachycardia in the patient is about the same as or similar to placebo. In some embodiments, the method results in orthostatic tachycardia in less than or equal to 5% of patients. In some embodiments, the method results in orthostatic tachycardia in less than or equal to 4.2% of patients. In some embodiments, the method results in orthostatic tachycardia in less than or equal to 5% of patients during a 29-day treatment period. In some embodiments, the method results in orthostatic tachycardia in a percentage of patients that is no more than 2% greater than placebo.

In some embodiments, the patient has an elevated risk of orthostatic tachycardia from administration of an antipsychotic agent. In some embodiments, the patient has a history of orthostatic tachycardia from a prior antipsychotic therapy. In some embodiments, the patient is susceptible to orthostatic tachycardia from an antipsychotic agent that has direct affinity to dopamine D2 receptors.

In some embodiments, the patient is not actively monitored for orthostatic tachycardia. In some embodiments, the patient is not warned about orthostatic tachycardia. In some embodiments, the patient is not concurrently treated for orthostatic tachycardia.

Compound 1 is an antipsychotic agent with a non-D2 mechanism of action, which shows broad efficacy in animal models of psychosis and depression. As described in the Examples below, Compound 1 shows efficacy in the treatment of schizophrenia. Specifically, the efficacy measures of Positive and Negative Symptom Scale (PANSS) total score, PANSS subscores (negative, positive, general psychopathology), Clinical Global Impressions-Severity (CGI-S) score, Brief Negative Symptom Scale (BNSS) total score, and Montgomery-Asberg Depression Rating Scale (MADRS) total score each showed an improvement (e.g., compared to placebo) in patients suffering from an acute exacerbation of schizophrenia treated with Compound 1.

Accordingly, in some embodiments, the methods of the present disclosure result in one or more of:

a reduction from baseline in PANSS total score of at least 17.2;

an effect size in PANSS total score of at least 0.45;

a reduction from baseline in PANSS positive subscale score of at least 5.5;

an effect size in PANSS positive subscale score of at least 0.32;

a reduction from baseline in PANSS negative subscale score of at least 3.1;

an effect size in PANSS negative subscale score of at least 0.37;

a reduction from baseline in PANSS general psychopathology subscale score of at least 9;

an effect size in PANSS general psychopathology subscale score of at least 0.51;

a reduction from baseline in CGI-S score of at least 1;

an effect size in CGI-S score of at least 0.52;

a reduction from baseline in BNSS total score of at least 7.1;

an effect size in BNSS total score of at least 0.48;

a reduction from baseline in MADRS total score of at least 3.3; and/or

an effect size in MADRS total score of at least 0.32.

In some embodiments, a method described herein further comprises treating a symptom of insomnia, anxiety, or headache in the patient. In some embodiments, the risk of insomnia, anxiety, headache, or any combination thereof in the patient is less than placebo. In some embodiments, the method minimizes insomnia, anxiety, headache or any combination thereof.

In some embodiments, the symptom is insomnia. In some embodiments, the symptom is anxiety. In some embodiments, the symptom is headache. In some embodiments, a method described herein further comprises treating dizziness in the patient. In some embodiments, the risk of insomnia, anxiety, headaches, schizophrenia, somnolence, agitation, nausea, diarrhea and dyspepsia, individually and as a group, is not clinically significant (i.e., is less than, the same as, or about the same as or similar to placebo).

In some embodiments, administering Compound 1, or a pharmaceutically acceptable salt thereof, comprises a titration period and a treatment period. In some examples, a first dose of Compound 1, or a pharmaceutically acceptable salt thereof, is administered during a titration period, followed by a therapeutic dose of Compound 1, or a pharmaceutically acceptable salt thereof, administered during a therapeutic period. The titration dose is less than the therapeutic dose. In some examples, the titration dose is 50 mg per day and the therapeutic dose is 75 mg per day. In some embodiments, the titration period is 3 days, followed by a therapeutic period (e.g., beginning on day 4 and continuing, e.g., through day 29). In some embodiments, 50 mg of Compound 1, or a pharmaceutically acceptable salt thereof, is administered on days 1-3 and 75 mg of Compound 1, or a pharmaceutically acceptable salt thereof, is administered on days 4-29.

In some embodiments, the therapeutic dose can be down-titrated to a reduced dose. In some examples a 75 mg dose can be reduced to a 50 mg dose. In some embodiments, Compound 1, or a pharmaceutically acceptable salt thereof, is administered as a flexible dose of 50 mg daily or 75 mg daily.

In some embodiments, provided is a method of treating schizophrenia in a patient, comprising administering to the patient Compound 1, or a pharmaceutically acceptable salt thereof, daily at a first dose for 1 to 3 days, followed by administering to the patient Compound 1, or a pharmaceutically acceptable salt thereof, daily at a therapeutic dose, wherein the first dose is less than the therapeutic dose. In some embodiments, Compound 1, or a pharmaceutically acceptable salt thereof, is administered daily at the first dose on days 1-3, and Compound 1, or a pharmaceutically acceptable salt thereof, is administered daily at the therapeutic dose on days 4-29. In some embodiments, the first dose is 50 mg and the therapeutic dose is 75 mg. In some embodiments, Compound 1, or a pharmaceutically acceptable salt thereof, is administered once daily. In some embodiments, Compound 1, or a pharmaceutically acceptable salt thereof, is administered orally.

In some embodiments, provided is a method of treating schizophrenia in a patient, comprising administering to the patient 50 mg daily of Compound 1, or a pharmaceutically acceptable salt thereof. In some embodiments, Compound 1, or a pharmaceutically acceptable salt thereof, is administered once daily. In some embodiments, Compound 1, or a pharmaceutically acceptable salt thereof, is administered daily for a 29-day treatment period. In some embodiments, Compound 1, or a pharmaceutically acceptable salt thereof, is administered orally.

In some embodiments, provided is a method of treating schizophrenia in a patient, comprising:
orally administering or having administered to the patient 75 mg daily of Compound 1, or a pharmaceutically acceptable salt thereof, during a treatment period;
determining or having determined if the patient has experienced an adverse event during the treatment period; and
reducing or having reduced administration to 50 mg daily of Compound 1, or a pharmaceutically acceptable salt thereof, if the patient experiences an adverse event during the treatment period.

In some embodiments, the method further comprises monitoring the patient for an adverse event during the treatment period.

In some embodiments, provided is a method of treating schizophrenia in a patient, comprising orally administering to the patient a therapeutically effective amount of Compound 1, or a pharmaceutically acceptable salt thereof, to achieve a maximum plasma concentration of Compound 1, or a pharmaceutically acceptable salt thereof, in the patient at 1-4 hours after a single dose and at 2-4 hours after multiple doses, wherein the therapeutically effective amount is 50 mg or 75 mg daily.

In some embodiments, provided is a method of treating schizophrenia in a patient, comprising orally administering to the patient a therapeutically effective amount of Compound 1, or a pharmaceutically acceptable salt thereof, to achieve a steady-state plasma concentration of Compound 1, or a pharmaceutically acceptable salt thereof, in a patient within 7 days, wherein the therapeutically effective amount is 50 mg or 75 mg daily.

In some embodiments, provided is a method of treating a symptom of insomnia, anxiety, or headache, in a patient having schizophrenia, comprising administering to the patient a therapeutically effective amount of Compound 1, or a pharmaceutically acceptable salt thereof. In some embodiments, the symptom is insomnia. In some embodiments, the symptom is anxiety. In some embodiments, the symptom is headache.

In some embodiments, provided is a method of treating insomnia, anxiety, or headache, associated with schizophrenia in a patient, comprising administering to the patient a therapeutically effective amount of Compound 1, or a pharmaceutically acceptable salt thereof.

In some embodiments, provided is a method of treating schizophrenia in a patient, comprising administering to the patient a therapeutically effective amount of Compound 1, or a pharmaceutically acceptable salt thereof, wherein the incidence of insomnia, anxiety, or headache, or any combination thereof in patients is less than placebo.

In some embodiments, provided is a method of reducing PANSS total score in a patient suffering from schizophrenia, comprising administering to the patient a therapeutically effective amount of Compound 1, or a pharmaceutically acceptable salt thereof, wherein the method results in (i) a reduction from baseline in PANSS total score of at least 17.2 or (ii) an effect size in PANSS total score of at least 0.45.

In some embodiments, provided is a method of reducing CGI-S score in a patient suffering from schizophrenia, comprising administering to the patient a therapeutically effective amount of Compound 1, or a pharmaceutically acceptable salt thereof, which produces (i) a reduction from baseline in CGI-S score of at least 1 or (ii) an effect size in CGI-S score of at least 0.52.

In some embodiments, provided is a method of reducing BNSS total score in a patient suffering from schizophrenia, comprising administering to the patient a therapeutically effective amount of Compound 1, or a pharmaceutically acceptable salt thereof, which produces (i) a reduction from baseline in BNSS total score of at least 7.1 or (ii) an effect size in BNSS total score of at least 0.48.

In some embodiments, provided is a method of reducing MADRS total score in a patient suffering from schizophrenia, comprising administering to the patient a therapeutically effective amount of Compound 1, or a pharmaceutically acceptable salt thereof, which produces (i) a reduction from baseline in MADRS total score of at least 3.3 or (ii) an effect size in MADRS total score of at least 0.32.

In some embodiments, Compound 1, or a pharmaceutically acceptable salt thereof, may be administered as part of a pharmaceutical composition. Pharmaceutical compositions of the present disclosure may be administered orally, parenterally, by inhalation, topically, rectally, nasally, buccally, sublingually, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

In some embodiments, the compositions are administered orally, intraperitoneally or intravenously. Sterile injectable forms of the compositions of the present disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, such as, for example, as a solution in 1,3-butanediol.

In some embodiments, pharmaceutically acceptable compositions of this disclosure may be orally administered in any orally acceptable dosage form including capsules, tablets, aqueous suspensions or solutions.

In some embodiments, the pharmaceutical compositions of the present disclosure comprise one or more pharmaceutically acceptable excipients, including one or more binders, bulking agents, buffers, stabilizing agents, surfactants, wetting agents, lubricating agents, diluents, disintegrants, viscosity enhancing or reducing agents, emulsifiers, suspending agents, preservatives, antioxidants, opaquing agents, glidants, processing aids, colorants, sweeteners, taste-masking agents, perfuming agents, flavoring agents, diluents, polishing agents, polymer matrix systems, plasticizers and other known additives to provide an elegant presentation of the drug or aid in the manufacturing of a medicament or pharmaceutical product comprising a composition of the present disclosure. Examples of carriers and excipients well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005.

In some embodiments, non-limiting examples of excipients include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), hydroxypropyl cellulose, titanium dioxide, talc, calcium carbonate (e.g., granules or powder), microcrystalline cellulose, powdered cellulose, dextrates, kaolin, silicic acid, sorbitol, starch, pre-gelatinized starch, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), colorants and mixtures thereof.

In some embodiments, pharmaceutical compositions are formulated with one or more pharmaceutically acceptable excipients in accordance with known and established practice. Thus, in some embodiments the compositions are formulated as, for example, a liquid, powder, elixir, injectable solution, or suspension.

In some embodiments, formulations for oral use may be provided as tablets, caplets, or capsules, wherein the pharmacologically active ingredients are mixed with an inert solid diluent.

In some embodiments, the oral dosage form is a solid oral dosage form. In some embodiments the solid oral dosage form comprises a tablet, and in some embodiments the solid oral dosage form comprises a capsule. Tablets may also include granulating and disintegrating agents, and may be coated or uncoated.

In some embodiments, formulations for topical use may be provided, for example as topical solutions, lotions, creams, ointments, gels, foams, patches, powders, solids, sponges, tapes, vapors, pastes or tinctures.

In some embodiments, a suitable daily dose of Compound 1, or a pharmaceutically acceptable salt thereof, will be that amount of the compound which is the lowest dose effective to produce a therapeutic effect. Such an effective dose will generally depend upon the factors described herein, or as understood by one having ordinary skill in the art. Generally, oral, intravenous and subcutaneous doses of Compound 1, or a pharmaceutically acceptable salt thereof, for a patient will range from about 0.005 mg per kilogram to about 5 mg per kilogram of body weight per day. In some embodiments, the oral dose of Compound 1, or a pharmaceutically acceptable salt thereof, will range from about 0.125 mg per kilogram of body weight to about 2.5 mg per kilogram of body weight per day. In some embodiments, the oral dose of Compound 1, or a pharmaceutically acceptable salt thereof, will range from about 0.25 mg per kilogram of body weight to about 2.5 mg per kilogram of body weight per day. In some embodiments, the oral dose of Compound 1, or a pharmaceutically acceptable salt thereof, will range from about 0.125 mg per kilogram of body weight to about 1.125 mg per kilogram of body weight per day. In some embodiments, the oral dose of Compound 1, or a pharmaceutically acceptable salt thereof, will range from about 10 mg to about 300 mg per day. In another embodiment, the oral dose of Compound 1, or a pharmaceutically acceptable salt thereof, will range from about 20 mg to about 250 mg per day. In another embodiment, the oral dose of Compound 1, or a pharmaceutically acceptable salt thereof, will range from about 100 mg to about 300 mg per day. In another embodiment, the oral dose of Compound 1, or a pharmaceutically acceptable salt thereof, will range from about 10 mg to about 100 mg per day. In another embodiment, the oral dose of Compound 1, or a pharmaceutically acceptable salt thereof, will range from about 50 mg to about 75 mg per day. In another embodiment, the oral dose of Compound 1, or a pharmaceutically acceptable salt thereof, will range from about 50 mg to about 200 mg per day. Each of the above-recited dosage ranges may be formulated as a single or multiple unit dosage formulations.

In some embodiments, Compound 1, or a pharmaceutically acceptable salt thereof, is administered orally. In some embodiments, Compound 1, or a pharmaceutically acceptable salt thereof, is administered daily. In some embodiments, Compound 1, or a pharmaceutically acceptable salt thereof, is administered at about 50 mg or about 75 mg per day.

In some embodiments, the method achieves a maximum plasma concentration of Compound 1, or a pharmaceutically acceptable salt thereof, in the patient at 1-4 hours after a single oral dose and at 2-4 hours after multiple oral doses. In some embodiments, the method achieves a maximum plasma concentration of Compound 1, or a pharmaceutically acceptable salt thereof, in the patient at 1-4 hours after a single oral dose. In some embodiments, the method achieves a maximum plasma concentration of Compound 1, or a pharmaceutically acceptable salt thereof, in the patient at 2-4 hours after multiple oral doses.

In some embodiments, the method achieves a steady-state plasma concentration of Compound 1, or a pharmaceutically acceptable salt thereof, in the patient within 7 days.

In some embodiments, Compound 1, or a pharmaceutically acceptable salt thereof, is administered daily during a 29-day treatment period.

In some embodiments, Compound 1, or a pharmaceutically acceptable salt thereof, may be used in combination with one or more second active agents to treat, prevent, and/or manage diseases and disorders described herein.

Some embodiments of the disclosure include methods of treating neurological and psychiatric diseases and disorders comprising administering to a patient a therapeutically effective amount of Compound 1, or a pharmaceutically acceptable salt thereof. Some embodiments include methods of preventing or managing neurological and psychiatric diseases and disorders comprising administering to a patient a therapeutically effective amount of Compound 1, or a pharmaceutically acceptable salt thereof, to prevent or manage the disease.

The Diagnostic and Statistical Manual of Mental Disorders, Fifth Ed., hereinafter, the “DSM-5”), published by the American Psychiatric Association in 2013, provides a standard diagnostic system upon which persons of skill rely for diagnosis of various diseases and disorders.

The term “mood disorder” as used herein includes depression, major depression, major depressive disorder, mild depression, severe depression without psychosis, severe depression with psychosis, melancholia (formerly endogenous depression), atypical depression, dysthymic disorder, manic depression, bipolar disorder, bipolar depression, bipolar I disorder, bipolar II disorder, bipolar III disorder, cyclothymic disorder, and chronic hypomania.

Psychiatric disorders are pathological conditions of the brain characterized by identifiable symptoms that result in abnormalities in cognition, emotion or mood, or the highest integrative aspects of behavior. These disorders may vary in severity of symptoms, duration, and functional impairment. Psychiatric disorders afflict millions of people worldwide resulting in tremendous human suffering and economic burden due to lost productivity. Mood disorders are a type of psychiatric disorder often defined as a group of heterogeneous, typically recurrent illnesses including unipolar (depressive) and bipolar (manic-depressive) disorders characterized by pervasive mood disturbances, psychomotor dysfunction, and vegetative symptoms. Suicide, the most serious complication in patients with mood disorders, is the cause of death in 15 to 25% of untreated patients with mood disorders; unrecognized or inadequately treated depression contributes to 50 to 70% of all completed suicides.

In some embodiments, the neurological disorder is: depression (e.g., major depressive disorder or dysthymia); bipolar disorder, seasonal affective disorder; cognitive deficit; fibromyalgia; pain (e.g., neuropathic pain); sleep related disorder (e.g., sleep apnea, insomnia, narcolepsy, cataplexy) including those sleep disorders which are produced by psychiatric conditions; chronic fatigue syndrome; attention deficit disorder (ADD); attention deficit hyperactivity disorder (ADHD); restless leg syndrome; schizophrenia; anxieties (e.g., general anxiety disorder, social anxiety disorder, panic disorder); obsessive compulsive disorder; post-traumatic stress disorder; seasonal affective disorder (SAD); premenstrual dysphoria; post-menopausal vasomotor symptoms (e.g., hot flashes, night sweats); neurodegenerative disease (e.g., Parkinson's disease, Alzheimer's disease and amyotrophic lateral sclerosis); manic disorder; dysthymic disorder; cyclothymic disorder; obesity; and substance abuse or dependency (e.g., cocaine addiction, nicotine addiction). In another embodiment, Compound 1, or a pharmaceutically acceptable salt thereof, is useful to treat, prevent, and/or manage two or more conditions/disorders, which are co-morbid, such as psychosis and depression.

Neurological disorders may also include cerebral function disorders, including without limitation, senile dementia, Alzheimer's type dementia, cognition, memory loss, amnesia/amnestic syndrome, epilepsy, disturbances of consciousness, coma, lowering of attention, speech disorder, Lennox syndrome, autism, and hyperkinetic syndrome.

In some embodiments, the disease or disorder which the methods of the present disclosure treat comprises one of more of a mood disorder, bipolar disorder (BPD), bipolar depression, sleep disorders, REM behavior disorder, psychosis disorders, Alzheimer's disease with agitation and/or psychosis, Parkinson's disease psychosis, schizophrenia, attenuated psychosis syndrome, prodromal schizophrenia, and schizoaffective disorder.

In some embodiments, the neurological or psychiatric disease or disorder is one or more of a mood disorder, bipolar disorder (BPD), bipolar depression, sleep disorders, REM behavior disorder, psychosis disorders, Alzheimer's disease with agitation and/or psychosis, Parkinson's disease psychosis, schizophrenia, attenuated psychosis syndrome, prodromal schizophrenia, and schizoaffective disorder.

In some embodiments, the neurological or psychiatric disease or disorder is selected from a psychosis, including schizophrenia (paranoid, disorganized, catatonic or undifferentiated), schizophreniform disorder, schizoaffective disorder, delusional disorder, brief psychotic disorder, shared psychotic disorder, psychoaffective disorder, aggression, delirium, Parkinson's psychosis, excitative psychosis, psychotic disorder due to a general medical condition, substance-induced or drug-induced (e.g., phencyclidine, ketamine and other dissociative anesthetics, amphetamine and other psychostimulants and cocaine) psychosis disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both positive, negative, and cognitive symptoms of schizophrenia and other psychoses; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); and Alzheimer's disease with agitation and/or psychosis.

In some embodiments, the neurological or psychiatric disease or disorder is selected from a depressive disorder including, but not limited to, unipolar depression, seasonal depression, post-partum depression, atypical depression, catatonic depression, elderly depression, endogenous depression, melancholic depression, perinatal depression, situational depression, chronic depression, bipolar depression, major depressive disorder (MDD), major depressive disorder with mixed features (MDD-MF), treatment resistant depression (TRD), and dysthymia, and are associated with depressed mood (sadness), poor concentration, insomnia, fatigue, appetite disturbances, excessive guilt and thoughts of suicide, premenstrual syndrome (PMS) and premenstrual dysphoric disorder (PDD), mood disorders due to a general medical condition, and substance-induced mood disorders.

In some embodiments, the neurological or psychiatric disease or disorder is selected from a bipolar disorder including, but not limited to, bipolar depression, bipolar I disorder, bipolar II disorder, cyclothymic disorder, substance/medication-induced bipolar and related disorders, bipolar and related disorder due to another medical condition, other specified bipolar and related disorder, and unspecified bipolar and related disorders.

In some embodiments, the neurological or psychiatric disease or disorder is selected from an eating disorder including, but not limited to, eating disorders such as obesity, bulimia nervosa, pica and compulsive eating disorders.

In some embodiments, the neurological or psychiatric disease or disorder is selected from a sleep disorder including, but not limited to, insomnia, disturbed sleep, jet lag, hypersomnia, cataplexy, sleep apnea, obstructive sleep apnea, REM sleep behavior disorder, Restless Leg Syndrome, periodic limb movement disorder, circadian rhythm sleep disorders, delayed sleep phase disorder, sleepwalking, night terrors, bed wetting, rapid eye movement sleep behavior disorder, shift work sleep disorder, excessive daytime sleepiness, non-24-hour sleep-wake disorder, sleep paralysis and narcolepsy.

In some embodiments, the neurological or psychiatric disease or disorder is a bipolar disorder. Bipolar disorders (including both bipolar I and bipolar II) are serious psychiatric disorders that have a prevalence of approximately 2% of the population and affect both genders alike. They are relapsing-remitting conditions characterized by cycling between elevated (i.e., manic) and depressed moods, which distinguishes them from other disorders such as major depressive disorder and schizophrenia.

Bipolar I is defined by the occurrence of a full manic episode, although most individuals experience significant depression. Symptoms of mania include elevated or irritable mood, hyperactivity, grandiosity, decreased need for sleep, racing thoughts, and in some cases, psychosis. The depressive episodes are characterized by anhedonia, sad mood, hopelessness, poor self-esteem, diminished concentration and lethargy. Bipolar II is defined as the occurrence of a major depressive episode and hypomanic (less severe mania) episode although patients spend considerably more time in the depressive state. Other related conditions include cyclothymic disorder.

In bipolar II disorder, depressive episodes alternate with hypomanias (relatively mild, nonpsychotic periods of usually <1 week). During the hypomanic period, mood brightens, the need for sleep decreases, and psychomotor activity accelerates beyond the patient's usual level. Often, the switch is induced by circadian factors (e.g., going to bed depressed and waking early in the morning in a hypomanic state). Hypersomnia and overeating are characteristic and may recur seasonally (e.g., in autumn or winter); insomnia and poor appetite occur during the depressive phase. For some persons, hypomanic periods are adaptive because they are associated with high energy, confidence, and supernormal social functioning. Many patients who experience pleasant elevation of mood, usually at the end of a depression, do not report it unless specifically questioned.

Patients with major depressive episodes and a family history of bipolar disorders (unofficially called bipolar III) often exhibit subtle hypomanic tendencies; their temperament is termed hyperthymic (i.e., driven, ambitious, and achievement-oriented).

In cyclothymic disorder, less severe hypomanic and mini-depressive periods follow an irregular course, with each period lasting a few days. Cyclothymic disorder is commonly a precursor of bipolar II disorder. But it can also occur as extreme moodiness without being complicated by major mood disorders. In such cases, brief cycles of retarded depression accompanied by low self-confidence and increased sleep alternate with elation or increased enthusiasm and shortened sleep. In another form, low-grade depressive features predominate; the bipolar tendency is shown primarily by how easily elation or irritability is induced by antidepressants. In chronic hypomania, a form rarely seen clinically, elated periods predominate, with habitual reduction of sleep to <6 hours. Persons with this form are constantly overcheerful, self-assured, overenergetic, full of plans, improvident, overinvolved, and meddlesome; they rush off with restless impulses and accost people.

Accordingly, in some embodiments, the neurological or psychiatric disease or disorder is one or more of bipolar I disorder, bipolar II disorder, cyclothymic disorder, other specified bipolar and related disorder, or unspecified bipolar and related disorder, and bipolar I disorder or bipolar II disorder with the specifiers of anxious distress, with mixed features, with rapid cycling, with melancholic features, with atypical features, with mood-congruent psychotic features, with mood incongruent psychotic features, with catatonia, with peripartum onset, and/or with seasonal pattern. A recent article by Hu et al [Prim Care Companion CNS Disord. 2014; 16(2): PCC.13r01599] highlights that bipolar disorder, while commonly encountered in the primary care setting, is often misdiagnosed or undiagnosed. The DSM-5 attempts to capture the large proportion of patients with subsyndromal mixed symptoms with the inclusion of the mixed specifier.

In some embodiments, the neurological or psychiatric disease or disorder is a depressive disorder. Depressive disorders include, but are not limited to, unipolar depression, seasonal depression, post-partum depression, atypical depression, catatonic depression, elderly depression, endogenous depression, melancholic depression, perinatal depression, situational depression, chronic depression, bipolar depression, major depressive disorder (MDD), major depressive disorder with mixed features (MDD-MF), treatment resistant depression (TRD), and dysthymia, and are associated with depressed mood (sadness), poor concentration, insomnia, fatigue, appetite disturbances, excessive guilt and thoughts of suicide, premenstrual syndrome (PMS) and premenstrual dysphoric disorder (PDD), mood disorders due to a general medical condition, and substance-induced mood disorders.

Depression is an affective disorder, the pathogenesis of which cannot be explained by any single cause or theory. Unfortunately, treatment options for depressed patients who have suboptimal clinical responses to therapy with an antidepressant are limited. Approximately thirty percent (30%) of patients initiating antidepressant therapy show suboptimal or delayed clinical responses to the first-line antidepressant agents that are commonly used to treat depression.

Typically, if a patient exhibits suboptimal or delayed clinical response after several weeks of therapy with an antidepressant, the clinician's initial approach is to increase the dose of the antidepressant. If the patient's response remains unsatisfactory after increasing the dose, the most common approaches that many clinicians will pursue are: a) switching to another antidepressant; or b) adding a second antidepressant; or c) attempting an augmentation therapy by administering agents such as lithium carbonate, thyroid hormone (triiodothyronine), psychostimulants, modafinil, atypical antipsychotics, buspirone, or pindolol.

In its full syndromal expression, clinical depression manifests as major depressive disorder, with episodic course and varying degrees of residual manifestations between episodes. The mood is typically depressed, irritable, and/or anxious. The patient may appear miserable, with furrowed brows, downturned corners of the mouth, slumped posture, poor eye contact, and monosyllabic (or absent) speech. The morbid mood may be accompanied by preoccupation with guilt, self-denigrating ideas, decreased ability to concentrate, indecisiveness, diminished interest in usual activities, social withdrawal, helplessness, hopelessness, and recurrent thoughts of death and suicide. Sleep disorders are common. In some, the morbid mood is so deep that tears dry up; the patient complains of an inability to experience usual emotions—including grief, joy, and pleasure—and of a feeling that the world has become colorless, lifeless, and dead.

Melancholia (formerly endogenous depression) is characterized by marked psychomotor slowing (of thinking and activity) or agitation (e.g., restlessness, wringing of the hands, pressure of speech), weight loss, irrational guilt, and loss of the capacity to experience pleasure. Mood and activity vary diurnally, with a nadir in the morning. Most melancholic patients complain of difficulty falling asleep, multiple arousals, and insomnia in the middle of the night or early morning. Sexual desire is often diminished or lost. Amenorrhea can occur. Anorexia and weight loss may lead to emaciation and secondary disturbances in electrolyte balance.

In atypical depression, reverse vegetative features dominate the clinical presentation; they include anxious-phobic symptoms, evening worsening, initial insomnia, hypersomnia that often extends into the day, and hyperphagia with weight gain. Unlike patients with melancholia, those with atypical depression show mood brightening to potentially positive events but often crash into a paralyzing depression with the slightest adversity. Atypical depressive and bipolar II disorders overlap considerably.

In dysthymic disorder, depressive symptoms typically begin insidiously in childhood or adolescence and pursue an intermittent or low-grade course over many years or decades; major depressive episodes may complicate it (double depression). In pure dysthymia, depressive manifestations occur at a subthreshold level and overlap considerably with those of a depressive temperament: habitually gloomy, pessimistic, humorless, or incapable of fun; passive and lethargic; introverted; skeptical, hypercritical, or complaining; self-critical, self-reproaching, and self-derogatory; and preoccupied with inadequacy, failure, and negative events.

Thorough evaluation of many persons with depression reveals bipolar traits, and as many as one in five patients with a depressive disorder also develops frank hypomania or mania. Most switches from unipolar to bipolar disorder occur within 5 years of the onset of depressive manifestations. Predictors of a switch include early onset of depression (<25 years old), postpartum depression, frequent episodes of depression, quick brightening of mood with somatic treatments (e.g., antidepressants, phototherapy, sleep deprivation, electroconvulsive therapy), and a family history of mood disorders for three consecutive generations.

Between episodes, patients with bipolar disorder exhibit depressive moodiness and sometimes high-energy activity; disruption in developmental and social functioning in bipolar depression is more common than in unipolar disorder. In bipolar disorder, depression episodes are shorter (3 to 6 months), age of onset is younger, onset of episodes is more abrupt, and cycles (time from onset of one episode to that of the next) are shorter than in unipolar disorder. Cyclicity is particularly accentuated in rapid-cycling forms of bipolar disorder (usually defined as >=4 episodes/year). In addition depressive episodes in bipolar disorder are a difficult component of BPD to treat. For example, psychiatrists indicate that about 25% of patients across all bipolar disorders are refractory during a manic episode, while about 70% are refractory during a depressive episode.

Accordingly, in some embodiments, the neurological or psychiatric disease or disorder is one or more of bipolar depression, major depressive disorder (MDD), persistent depressive disorder (Dysthymia), premenstrual dysphoric disorder (PMDD), major depressive disorder with mixed features (MDD-MF), depressive disorder due to another medical condition, other specified depressive disorder, unspecified depressive disorder, or treatment resistant depression (TRD), and MDD with the specifiers of anxious distress, with mixed features, with melancholic features, with atypical features, with mood-congruent psychotic features, with mood-incongruent psychotic features, with catatonia, with peripartum onset, and/or with seasonal pattern, and seasonal affective disorder.

It is to be understood that TRD is a term used in clinical psychiatry to describe cases of major depressive disorder (MDD) that do not respond adequately to appropriate courses of at least two antidepressants.

In some embodiments, a depressive disorder is associated with acute suicidality or suicide ideation. The United States Food and Drug Administration has adopted a “black box” label warning indicating that antidepressants may increase the risk of suicidal thinking and behavior in some children, adolescents and young adults (up to age 24) with a depressive disorder such as MDD. In some embodiments, the compositions and methods of the present disclosure do not increase the risk of suicidal thinking and/or behavior in children, adolescents and/or young adults with a depressive disorder, e.g., with MDD. In some embodiments, the present disclosure provides medicaments for and provides methods of treating one or more symptoms of a depressive disorder (e.g., MDD) in children, adolescents and/or young adults without increasing the risk of suicidal thinking and/or behavior.

In some embodiments, the neurological or psychiatric disease or disorder is schizophrenia. Schizophrenia is a disorder of unknown origin, which usually appears for the first time in early adulthood and is marked by characteristics such as psychotic symptoms, phasic progression and development, and/or deterioration in social behavior and professional capability. Characteristic psychotic symptoms are disorders of thought content (e.g., multiple, fragmentary, incoherent, implausible or simply delusional contents, or ideas of persecution) and of mentality (e.g., loss of association, flight of imagination, incoherence up to incomprehensibility), as well as disorders of perceptibility (e.g., hallucinations), emotions (e.g., superficial or inadequate emotions), self-perceptions, intentions, impulses, and/or inter-human relationships, and psychomotoric disorders (e.g., catatonia). Other symptoms are also associated with this disorder. Schizophrenia is classified into subgroups: the paranoid type, characterized by delusions and hallucinations and absence of thought disorder, disorganized behavior, and affective flattening; the disorganized type, also named “hebephrenic schizophrenia,” in which thought disorder and flat affect are present together; the catatonic type, in which prominent psychomotor disturbances are evident, and symptoms may include catatonic stupor and waxy flexibility; and the undifferentiated type, in which psychotic symptoms are present but the criteria for paranoid, disorganized, or catatonic types have not been met. The symptoms of schizophrenia normally manifest themselves in three broad categories: positive, negative and cognitive symptoms. Positive symptoms are those which represent an “excess” of normal experiences, such as hallucinations and delusions. Negative symptoms are those where the patient suffers from a lack of normal experiences, such as anhedonia and lack of social interaction. The cognitive symptoms relate to cognitive impairment in schizophrenics, such as lack of sustained attention and deficits in decision making.

Accordingly, in some embodiments, the neurological or psychiatric disease or disorder is one or more of schizotypal (personality) disorder, delusional disorder, brief psychotic disorder, schizophreniform disorder, schizophrenia, schizoaffective disorder, substance/medication-induced psychotic disorder, psychotic disorder due to another medical condition, other specified schizophrenia spectrum and other psychotic disorder, unspecified schizophrenia spectrum, and other psychotic disorder.

It is to be understood that schizoaffective disorder includes a condition that includes aspects of both schizophrenia and a mood disorder, such as, for example, a major depressive disorder, a bipolar disorder, etc.

In some embodiments, the neurological or psychiatric disease or disorder is anxiety disorder. Anxiety disorders are characterized by fear, worry, and uneasiness, usually generalized and unfocused as an overreaction to a situation. Anxiety disorders differ in the situations or types of objects that induce fear, anxiety, or avoidance behavior, and the associated cognitive ideation. Anxiety differs from fear in that anxiety is an emotional response to a perceived future threat while fear is associated with a perceived or real immediate threat. They also differ in the content of the associated thoughts or beliefs. Examples of anxiety disorders include separation anxiety disorder, selective mutism, specific phobia, social anxiety disorder (social phobia), panic disorder, panic attack specifier, agoraphobia, generalized anxiety disorder, substance/medication-induced anxiety disorder, anxiety disorder due to another medical condition, illness anxiety disorder, social (pragmatic) communication disorder, other specified anxiety disorder, and unspecified anxiety disorder; and stressor-related disorders, including reactive attachment disorder, disinhibited social engagement disorder, posttraumatic stress disorder (PTSD), acute stress disorder, and adjustment disorders.

In some embodiments, the neurological or psychiatric disease or disorder is a sleep disorder including those sleep disorders which are produced by psychiatric conditions, including, but not limited to, insomnia, disturbed sleep, jet lag, hypersomnia, cataplexy, sleep related disorder (e.g., sleep apnea, insomnia, narcolepsy, cataplexy), obstructive sleep apnea, REM sleep behavior disorder, Restless Leg Syndrome, periodic limb movement disorder, circadian rhythm sleep disorders, delayed sleep phase disorder, sleepwalking, night terrors, bed wetting, rapid eye movement sleep behavior disorder, shift work sleep disorder, excessive daytime sleepiness, non-24-hour sleep-wake disorder, sleep paralysis and narcolepsy.

[197] In some embodiments, the neurological or psychiatric disease or disorder is a social function disorder. In some embodiments, the social function disorder is a neurodevelopmental disorder, an obsessive-compulsive disorder or a disruptive, impulse-control and conduct disorder. In some embodiments, the social function disorder is a language disorder, a speech sound disorder, a childhood-onset fluency disorder (stuttering), a social communication disorder, a developmental coordination disorder, a stereotypical movement disorder, a tic disorder, Tourette's disorder, a persistent (chronic) motor or vocal tic disorder, a provisional tic disorder, another specified tic disorder, an unspecified tic disorder, an obsessive-compulsive disorder, or an impulse-control disorder. In some embodiments, the social function disorder is a language disorder, a speech sound disorder, a childhood-onset fluency disorder (stuttering), a social communication disorder, a developmental coordination disorder, a stereotypical movement disorder, a tic disorder, Tourette's disorder, a persistent (chronic) motor or vocal tic disorder, a provisional tic disorder, another specified tic disorder, or an unspecified tic disorder. In some embodiments, the social function disorder is a language disorder, a speech sound disorder, a childhood-onset fluency disorder (stuttering), or a social communication disorder. In some embodiments, the social function disorder is a language disorder, childhood-onset fluency disorder (stuttering), social communication disorder, developmental coordination disorder, stereotypical movement disorder, persistent (chronic) motor or vocal tic disorder, provisional tic disorder, other specified tic disorder, or unspecified tic disorder.

### EXAMPLES

### Example 1: 4-Week Clinical Study

Compound 1 was evaluated in human patients to study its efficacy and safety in the treatment of schizophrenia in a 4-week, randomized, placebo-controlled trial. Hospitalized patients aged 18 to 40 years of age were eligible for enrollment if they met DSM-5 criteria for schizophrenia and were experiencing an acute exacerbation of psychotic symptoms (PANSS total score≥80; item score≥4 on 2-or-more of delusions, conceptual disorganization, hallucinatory behavior, or unusual thought content). Patients were randomized, double-blind, to 4-weeks of flexible-dose treatment with the HCl salt of Compound 1 given orally once daily (doses of 50 or 75 mg). The primary efficacy endpoint was change from baseline to week 4 in the Positive and Negative Symptom Scale (PANSS) total score. Secondary efficacy endpoints included change from baseline to week 4 in the Clinical Global Impressions-Severity (CGI-S) score, PANSS subscale scores, the Brief Negative Symptom Scale (BNSS) total score and the Montgomery-Asberg Depression Rating Scale (MADRS) total score. Change from baseline in primary and secondary efficacy measures were analyzed using a mixed model for repeated measurement (MMRM) analysis.

Study Design:

Patients first underwent a screening/washout period for up to 14 days. Patients were randomized into placebo and treatment groups. The treatment group received 50 mg/day of Compound 1 for 3 days, followed by a flexible dose of 50 mg/day or 75 mg/day of Compound 1 on days 4-29. The placebo group received placebo for 29 days.

Key Inclusion Criteria:
Male and female, 18-40 years of age
Time since initial diagnosis of schizophrenia≥6 months
Time since current acute exacerbation of psychotic symptoms≤2 months
Prior hospitalizations for treatment of acute exacerbation of schizophrenia≤2
Screening and baseline PANSS total score≥80 and PANSS item score≥4 on two or more of: delusions (P1), conceptual disorganization (P2), hallucinatory behavior (P3), and unusual thought contents (G9)
Screening and baseline CGI-S score≥4

Study Endpoints:

Primary Endpoint:
Change from baseline in PANSS total score at week 4

Secondary Endpoints:
Change from baseline in CGI-S score at week 4
Change from baseline in PANSS subscale scores at week 4
Change from baseline in BNSS total score at week 4
Change from baseline in MADRS total score at week 4
The incidences of adverse events (AEs), serious adverse events (SAEs), and adverse events leading to discontinuation from study

Statistical Analysis Method:

The mixed-effect model for repeated measure (MMRM) was used. Change from baseline in PANSS total score was analyzed using an MMRM model, with fixed effects for treatment, visit (Day 4, Weeks 1-4); as a categorical variable), treatment-by-visit interaction, baseline PANSS total score, and pooled center. Centers were pooled by country. An unstructured covariance matrix was used to model the within-subject correlation. MMRM was also used to analyze the secondary endpoints.

Baseline Characteristics:

Baseline subject characteristics are shown in Table 1.

TABLE 1
Baseline subject characteristics

| | | Placebo | | Compound 1 | | Total | |
|---|---|---|---|---|---|---|---|
| | Statistic | (N = 125) | | (N = 120) | | (N = 245) | |
| PANSS Total Score | Mean ± SD | 99.7 | (7.76) | 101.4 | (8.40) | 100.5 | (8.11) |
| | Median | 100.0 | | 102.0 | | 101.0 | |
| | Min, Max | 80, 122 | | 82, 122 | | 80, 122 | |
| PANSS Positive | Mean ± SD | 25.4 | (3.05) | 25.8 | (3.30) | 25.6 | (3.17) |
| Subscale Score | Median | 25.0 | | 25.5 | | 25.0 | |
| | Min, Max | 17, 33 | | 18, 33 | | 17, 33 | |
| PANSS Negative | Mean ± SD | 24.9 | (3.97) | 24.7 | (3.93) | 24.8 | (3.94) |
| Subscale Score | Median | 25.0 | | 25.0 | | 25.0 | |
| | Min, Max | 10, 36 | | 15, 33 | | 10, 36 | |
| PANSS Positive vs. | | | | | | | |
| Negative Subscale Score | | | | | | | |
| Positive < Negative | n (%) | 57 | (45.6%) | 49 | (40.8%) | 106 | (43.3%) |
| Positive >= Negative | n (%) | 68 | (54.4%) | 71 | (59.2%) | 139 | (56.7%) |
| CGI-S Score | Mean (SD) | 4.9 | (0.48) | 5.0 | (0.44) | 5.0 | (0.46) |
| 4-5 | n (%) | 114 | (91.2%) | 107 | (89.2%) | 221 | (90.2%) |
| >5 | n (%) | 11 | (8.8%) | 13 | (10.8%) | 24 | (9.8%) |

Results:

In this randomized, placebo-controlled, 4-week study, Compound 1, in flexible doses of 50 or 75 mg/day, demonstrated statistically significant and clinically meaningful symptom improvement in patients with schizophrenia experiencing an acute exacerbation. Compound 1 exhibited robust, broad-spectrum activity across a range of positive, negative, depressive, and general psychopathology symptoms. Improvement in negative symptoms was especially notable, with an effect size of 0.48 on the Brief Negative Symptom Scale. The tolerability and safety profile of Compound 1 appeared to be similar to placebo in this 4-week trial.

Efficacy:

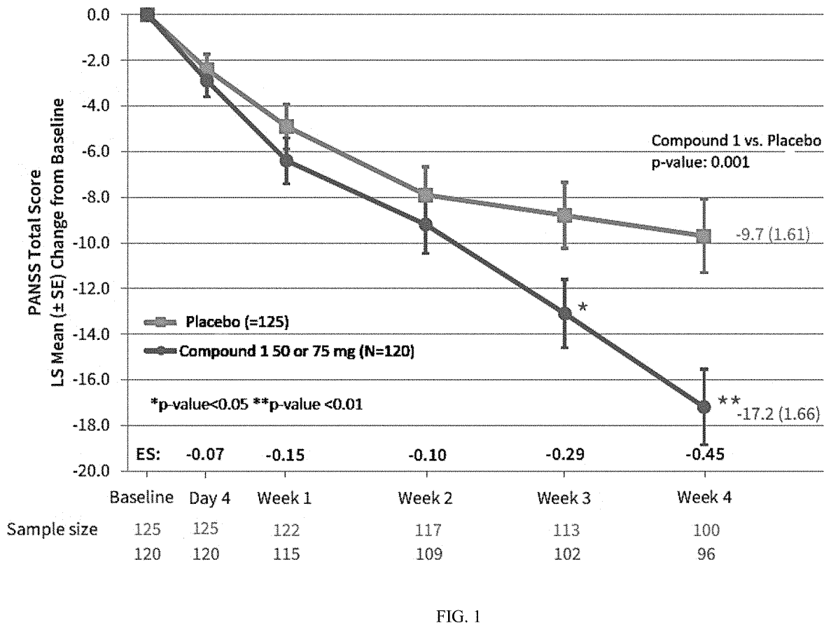

FIG. 1 shows the change from baseline in PANSS total score of patients during the 4-week study. The treatment group had a least squares mean change from baseline at week 4 of −17.2 compared to −9.7 for placebo, which corresponds to an effect size of 0.45.

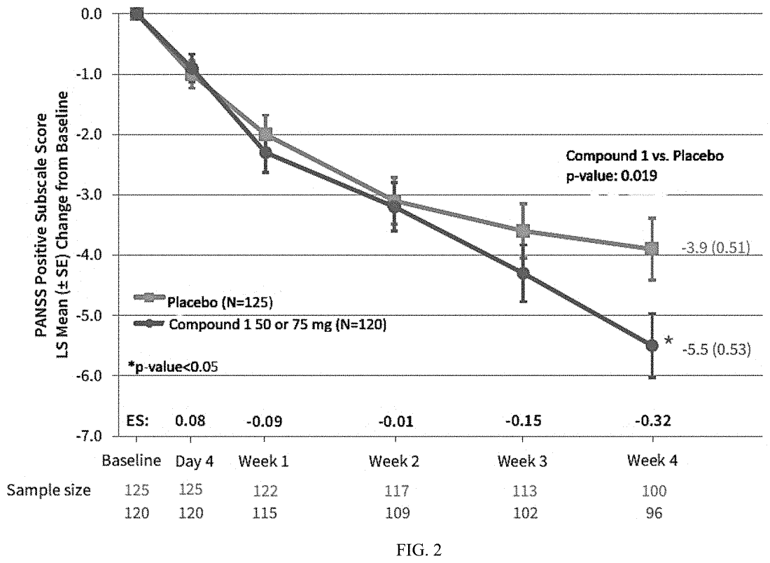

FIG. 2 shows the change from baseline in PANSS positive subscale score of patients during the 4-week study. The treatment group had a least squares mean change from baseline at week 4 of −5.5 compared to −3.9 for placebo, which corresponds to an effect size of 0.32.

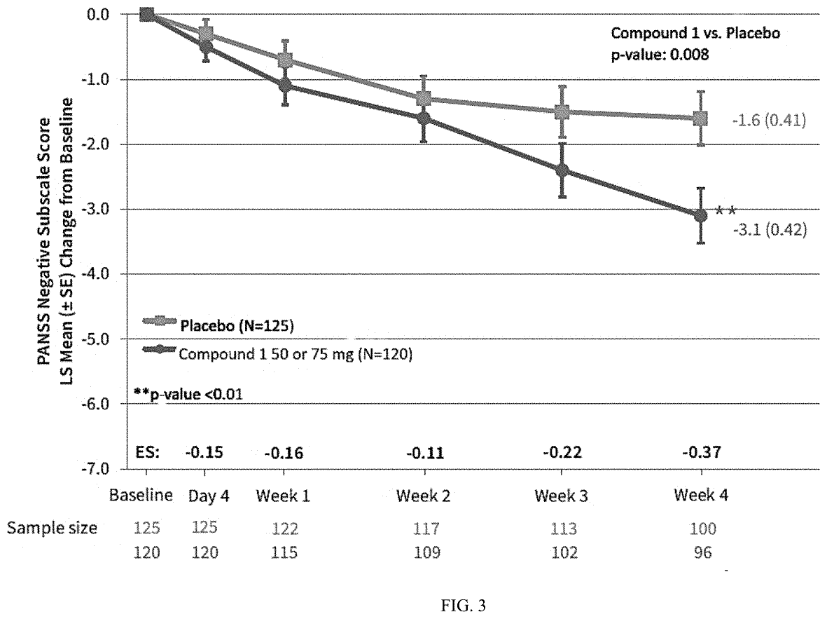

FIG. 3 shows the change from baseline in PANSS negative subscale score of patients during the 4-week study. The treatment group had a least squares mean change from baseline at week 4 of −3.1 compared to −1.6 for placebo, which corresponds to an effect size of 0.37.

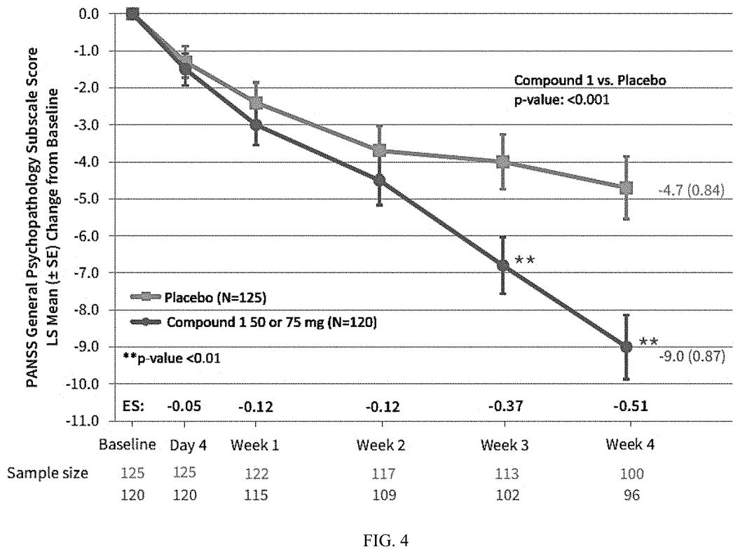

FIG. 4 shows the change from baseline in PANSS general psychopathology subscale score of patients during the 4-week study. The treatment group had a least squares mean change from baseline at week 4 of −9.0 compared to −4.7 for placebo, which corresponds to an effect size of 0.51.

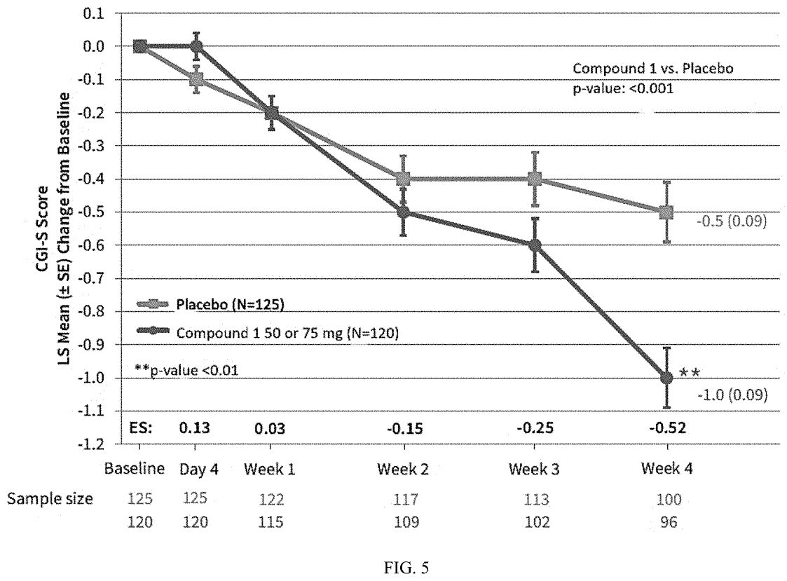

FIG. 5 shows the change from baseline in CGI-S score of patients during the 4-week study. The treatment group had a least squares mean change from baseline at week 4 of −1.0 compared to −0.5 for placebo, which corresponds to an effect size of 0.52.

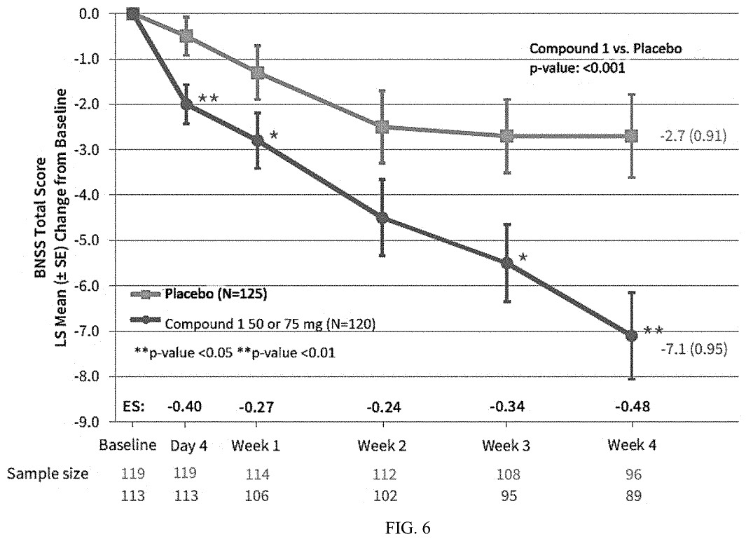

FIG. 6 shows the change from baseline in BNSS total score of patients during the 4-week study. The treatment group had a least squares mean change from baseline at week 4 of −7.1 compared to −2.7 for placebo, which corresponds to an effect size of 0.48.

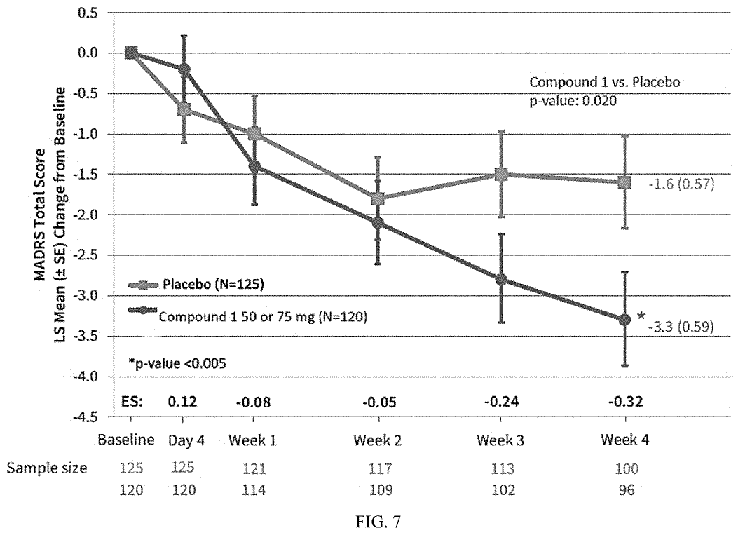

FIG. 7 shows the change from baseline in MADRS total score of patients during the 4-week study. The treatment group had a least squares mean change from baseline at week 4 of −3.3 compared to −1.6 for placebo, which corresponds to an effect size of 0.32.

Adverse Events:

Adverse events in the patients were monitored. Adverse events are untoward medical occurrences that started at the same time of or after the first dose of study medication. The incidence of adverse events in the treatment group was very low. Across all types of adverse events the incidence for the treatment group was similar to placebo. For certain adverse events, the incidence in the treatment group was less than placebo. The incidence of adverse events compares favorably to commercially available antipsychotic agents, including the atypical antipsychotics that have affinity to D2 dopamine receptors.

Table 2 summarizes the incidence of general adverse events occurring in >2% of patients in either the treatment group or placebo. The incidence of each of headache, insomnia, acute exacerbation of schizophrenia and anxiety was lower in the treatment group than in the placebo group.

TABLE 2
General adverse events

| | Placebo (N = 125) | Compound 1 (N = 120) |
|---|---|---|
| Preferred Term | n (%) | n (%) |
| Subjects with AE* | 63 (50.4%) | 55 (45.8%) |
| Headache | 15 (12.0%) | 11 (9.2%) |
| Schizophrenia | 10 (8.0%) | 8 (6.7%) |
| Somnolence | 6 (4.8%) | 8 (6.7%) |
| Agitation | 6 (4.8%) | 6 (5.0%) |
| Nausea | 4 (3.2%) | 6 (5.0%) |
| Insomnia | 13 (10.4%) | 4 (3.3%) |
| Diarrhea | 1 (0.8%) | 3 (2.5%) |
| Dyspepsia | 0 | 3 (2.5%) |
| Anxiety | 9 (7.2%) | 2 (1.7%) |

*Subjects with multiple AEs are counted only once.

Table 3 summarizes the incidence of extrapyramidal adverse events. Incidence of extrapyramidal adverse events in the treatment group was about the same as placebo.

TABLE 3
Extrapyramidal adverse events

| | Placebo | Compound 1 |
|---|---|---|
| | (N = 125) | (N = 120) |
| Preferred Term | n (%) | n (%) |
| Any subject experiencing an extrapyramidal | 4 (3.2%) | 4 (3.3%) |
| symptom AE* | | |
| Akathisia | 1 (0.8%) | 2 (1.7%) |
| Restlessness | 1 (0.8%) | 0 |
| Joint Stiffness | 1 (0.8%) | 0 |
| Musculoskeletal stiffness | 2 (1.6%) | 1 (0.8%) |
| Nuchal rigidity | 1 (0.8%) | 0 |
| Postural tremor | 0 | 1 (0.8%) |
| Tremor | 2 (1.6%) | 0 |

*Subjects with multiple AEs are counted only once.

Table 4 summarizes the incidence of cardiovascular adverse events. Incidence of cardiovascular adverse events in the treatment group were similar to placebo. Total of cardiovascular adverse events incidence in the treatment group was 4.2% compared to 4.0% for placebo.

TABLE 4
Cardiovascular adverse events

| | Placebo | Compound 1 |
|---|---|---|
| | (N = 125) | (N = 120) |
| SOC/Preferred Term | n (%) | n (%) |
| Cardiac Disorders | 2 (1.6%) | 3 (2.5%) |
| Atrial tachycardia | 0 | 1 (0.8%) |
| Bradycardia | 0 | 1 (0.8%) |
| Cardiovascular insufficiency* | 0 | 1 (0.8%) |
| Palpitations | 1 (0.8%) | 0 |
| Postural tachycardia syndrome | 1 (0.8%) | 0 |
| Vascular Disorders | 1 (0.8%) | 2 (1.7%) |
| Blood pressure increased | 0 | 1 (0.8%) |
| Hypertension | 0 | 1 (0.8%) |
| Hypotension | 0 | 1 (0.8%) |
| Hot flush | 1 (0.8%) | 0 |
| Dizziness | 2 (1.6%) | 0 |

*Cardiovascular insufficiency resulted in death.

Table 5 summarizes the incidence of serious adverse events. The incidence of serious adverse events in the treatment group was less than placebo.

TABLE 5
Serious adverse events

| | Placebo | | Compound 1 | |
|---|---|---|---|---|
| | (N = 125) | | (N = 120) | |
| Preferred Term | n (%) | # Events | n (%) | # Events |
| Subjects with any SAE* | 3 (2.4%) | 4 | 2 (1.7%) | 2 |
| Schizophrenia | 3 (2.4%) | 3 | 1 (0.8%) | 1 |
| Cardiovascular | 0 | 0 | 1 (0.8%) | 1 |
| insufficiency** | | | | |
| Suicide attempt | 1 (0.8%) | 1 | 0 | 0 |

*Subjects with multiple AEs are counted only once.
**Cardiovascular insufficiency resulted in death.

Table 6 summarizes the incidence of adverse events leading to discontinuation from study. The incidence of such adverse events was similar between treatment group and placebo.

TABLE 6
Adverse events leading to discontinuation

| | Placebo | | Compound 1 | |
|---|---|---|---|---|
| | (N = 125) | | (N = 120) | |
| | | # | | # |
| Preferred Term | n (%) | Events | n (%) | Events |
| Any AE leading to discon- | 8 (6.4%) | 8 | 11 (9.2%) | 11 |
| tinuation from study * | | | | |
| Schizophrenia | 6 (4.8%) | 6 | 8 (6.7%) | 8 |
| Psychotic disorder | 0 | 0 | 1 (0.8%) | 1 |
| Insomnia | 0 | 0 | 1 (0.8%) | 1 |
| Cardiovascular insuffi- | 0 | 0 | 1 (0.8%) | 0 |
| ciency** | | | | |
| Suicide attempt | 1 (0.8%) | 1 | 0 | 0 |
| Palpitations | 1 (0.8%) | 1 | 0 | 0 |

* Subjects with multiple AEs are counted only once.
**Cardiovascular insufficiency resulted in death.

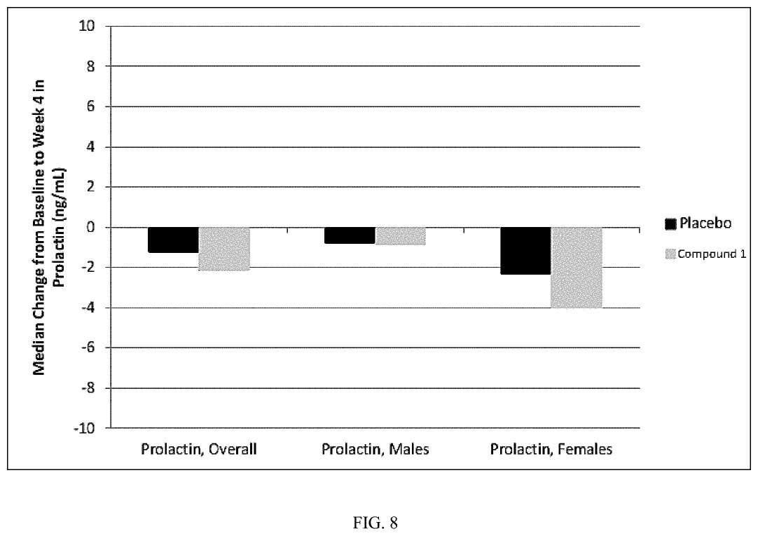

FIG. 8 shows the median change from baseline in prolactin levels at week 4. The treatment group on average experienced a decrease in prolactin. Table 7 summarizes the prolactin shifts from baseline at week 4. There was no clinically significant impact of Compound 1 on prolactin.

TABLE 7
Prolactin level change from baseline

| | Placebo | Compound 1 | |
|---|---|---|---|
| Prolactin | (N = 125) | (N = 120) | |
| Overall, n | 113 | 114 | |
| Low to normal | 2 (1.8%) | 5 | (4.4%) |
| Low to high | 0 | 0 | |
| Normal to high | 9 (8.0%) | 19 | (16.7%)* |
| Males, n (normal range: 2.64-13.13 | 71 | 74 | |
| ng/mL) | | | |
| Low to normal | 2 (2.8%) | 5 | (6.8%) |
| Low to high | 0 | 0 | |
| Normal to high | 9 (12.7%) | 16 | (21.6%) |
| Females, n (normal range: 2.74-26.72 | 42 | 40 | |
| ng/mL) | | | |
| Low to normal | 0 | 0 | |
| Low to high | 0 | 0 | |
| Normal to high | 0 | 3 | (7.5%) |

*Six of the subjects in the Compound 1 group who went from normal to high prolactin levels received another antipsychotic prior to final visit.

Table 8 summarizes the incidence of orthostatic hypotension and orthostatic tachycardia. Orthostatic hypotension is defined as a decrease of ≥20 mmHg in systolic blood pressure or ≥10 mmHg in diastolic blood pressure after the subject had been standing for at least 2 to 4 minutes, compared to the systolic blood pressure and diastolic pressure measured in the supine position, respectively. Orthostatic tachycardia is defined as a heart rate increase of ≥20 beats per minute (bpm) and a heart rate of >100 bpm after the subject was standing for at least 2 to 4 minutes, compared to the heart rate measured in the supine position. The incidence of orthostatic hypotension and orthostatic tachycardia in the treatment group was similar to placebo, with the incidence of orthostatic hypotension in the treatment group being less than placebo.

TABLE 8
Orthostatic hypotension and orthostatic tachycardia

| | Placebo | Compound 1 |
|---|---|---|
| | (N = 125) | (N = 120) |
| Orthostatic hypotension | 9 (7.2%) | 5 (4.2%) |
| ≥20 mmHg decrease in SBP from supine to | 7 (5.6%) | 2 (1.7%) |
| standing | | |
| ≥10 mm Hg decrease in DBP from supine to | 6 (4.8%) | 3 (2.5%) |
| standing | | |
| Orthostatic tachycardia (≤20 bpm increase | 3 (2.4%) | 5 (4.2%) |
| from supine to standing in heart rate | | |
| and >100 bpm after standing) | | |

Table 9 summarizes the incidence of QT prolongation as measured by the QTcF interval. Patient data was collected via electrocardiogram (ECG). The number and percentage of subjects with QTc values in the following categories were identified. The same criteria apply to both QTcF and QTcB.

>450 msec at any post-baseline time point (including unscheduled visits) not present at baseline

>480 msec at any post-baseline time point (including unscheduled visits) not present at baseline

>500 msec at any post-baseline time point (including unscheduled visits) not present at baseline

[symbol] 30 msec increase from baseline for at least one post-baseline measurement (including unscheduled visits) and <60 msec increase from baseline for all post-baseline measurements (including unscheduled visits)

[symbol] 60 msec increase from baseline for at least one post-baseline measurement (including unscheduled visits)

For both the treatment group and placebo group, there were no incidences of QT prolongation.

TABLE 9
QTcF interval

| | Placebo | Compound 1 |
|---|---|---|
| | (N = 125) | (N = 120) |
| QTcF Interval, n | 113 | 113 |
| >450 msec at any post-baseline time point not | 0 | 0 |
| present at baseline | | |
| >480 msec at any post-baseline time point not | 0 | 0 |
| present at baseline | | |
| >500 msec at any post-baseline time point not | 0 | 0 |
| present at baseline | | |
| ≥30 msec increase from baseline for at least | 0 | 0 |
| one post-baseline measurement and <60 msec | | |
| increase from baseline for all post-baseline | | |
| measurements | | |
| ≥60 msec increase from baseline for at least | 0 | 0 |
| one post-baseline measurement | | |

Table 10 summarizes the extrapyramidal symptoms as measured by the Barnes Akathisia Rating Scale (BARS), the Abnormal Involuntary Movement Scale (AIMS), and the Simpson-Angus Scale (SAS).

TABLE 10
AIMS, BARS, and SAS scores.

| | Placebo | | Compound 1 | |
|---|---|---|---|---|
| | (N = 125) | | (N = 120) | |
| AIMS Total Score Classification - Shift | 125 | | 120 | |
| from Baseline (Overall Post-Baseline), | | | | |
| n | | | | |
| Normal to Abnormal | 0 | | 2 | (1.7%) |
| AIMS Global Severity Score - | 125 | | 120 | |
| Categorical Summary of Change from | | | | |
| Baseline (LOCF Endpoint), n | | | | |
| Worsened | 1 | (0.8%) | 2 | (1.7%) |
| Unchanged | 124 | (99.2%) | 117 | (97.5%) |
| Improved | 0 | | 0 | |
| BARS Global Clinical Assessment - | 125 | | 120 | |
| Categorical Summary of Change from | | | | |
| Baseline (LOCF Endpoint), n | | | | |
| Worsened | 5 | (4.0%) | 1 | (0.8%) |
| Unchanged | 116 | (92.8%) | 107 | (89.2%) |
| Improved | 4 | (3.2%) | 12 | (10.0%) |
| SAS Mean Score Classification Shift | 125 | | 120 | |
| (Overall Post-Baseline) | | | | |
| Normal to Abnormal | 3 | (2.4%) | 2 | (1.7%) |

Accordingly, various methods of the present disclosure result in low incidence of adverse events, for example, adverse events less than, the same as, or about the same as or similar to placebo. This is in contrast to many typical and atypical antipsychotics, which have affinity to dopamine D2 receptors, and which produce higher incidence of adverse events.

### Example 2: 26-Week Extension Study

A 26-week open-label extension study was performed for subjects with schizophrenia who completed the treatment phase from Example 1. Patients who met the entry criteria transitioned immediately from the Example 1 study to the extension study. A total of 157 patients enrolled in the extension study. Patients were dosed orally once daily with the HCl salt of Compound 1 (referred to in the Tables as “Compound 1”) at 50 mg/day for Days 1-3 of the extension study and then at a flexible dose of 25, 50 or 75 mg/day through the remainder of the 26 weeks.

Safety and tolerability were monitored throughout the study by collection of physical examination results, ECGs, vital signs, AEs, clinical laboratory parameters, C-SSRS, body weight and BMI. Effectiveness was evaluated using the PANSS total and subscale scores, as well as CGI-S, BNSS, and MADRS scores. Subjects provided information on subjective drug effects via a questionnaire.

The primary endpoints of the study were the incidence of overall AEs, SAEs, and AEs leading to discontinuation. Secondary endpoints included:
Absolute values and changes from double-blind (DB) Baseline of Example 1 in clinical laboratory tests (hematology, serum chemistry, urinalysis, glucose and lipid panel, prolactin, glycosylated hemoglobin (HbA1c));
Absolute values and changes from DB Baseline of Example 1 in clinical evaluations (vital signs body weight, BMI, blood pressure [supine and standing], heart rate [supine and standing], 12 lead ECGs); and
Changes from DB Baseline of Example 1 (see Table 1) in PANSS total score, PANSS subscale scores (positive, negative, and general psychopathology), CGI-S score, BNSS total score, and MADRS total score.

Results:

105 subjects (66.9%) completed the 26-week study; 52 subjects (33.1%) discontinued due to adverse event (18; 11.5%), withdrawal by subject (16; 10.2%), other (9; 5.7%), lack of efficacy (8; 5.1%) or noncompliance (1; 0.6%).

Efficacy measures were recorded over the course of the 26-week extension study.

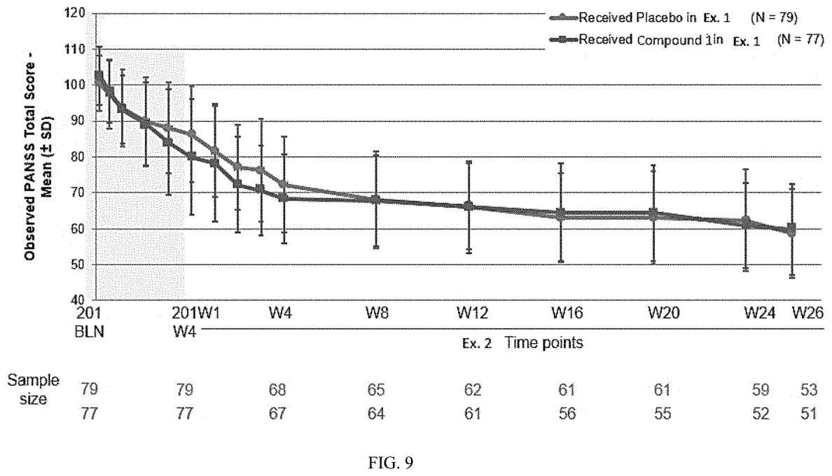

FIG. 9 shows the PANSS total score during the extension study with the PANSS total score data from the Example 1 study shown for reference.

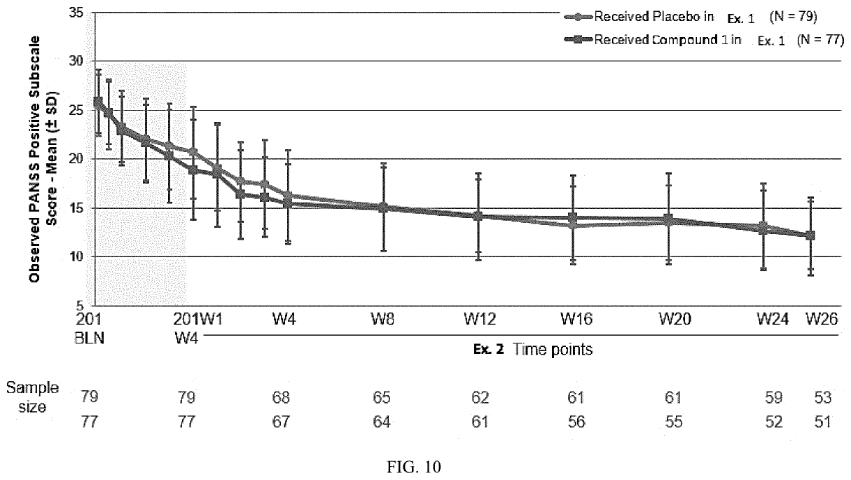

FIG. 10 shows the PANSS positive subscale score during the extension study with the PANSS positive subscale score data from the Example 1 study shown for reference.

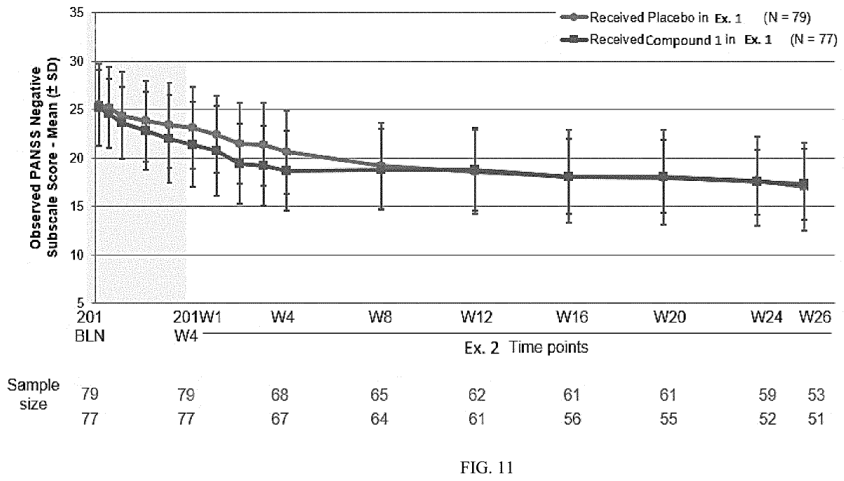

FIG. 11 shows the PANSS negative subscale score during the extension study with the PANSS negative subscale score data from the Example 1 study shown for reference.

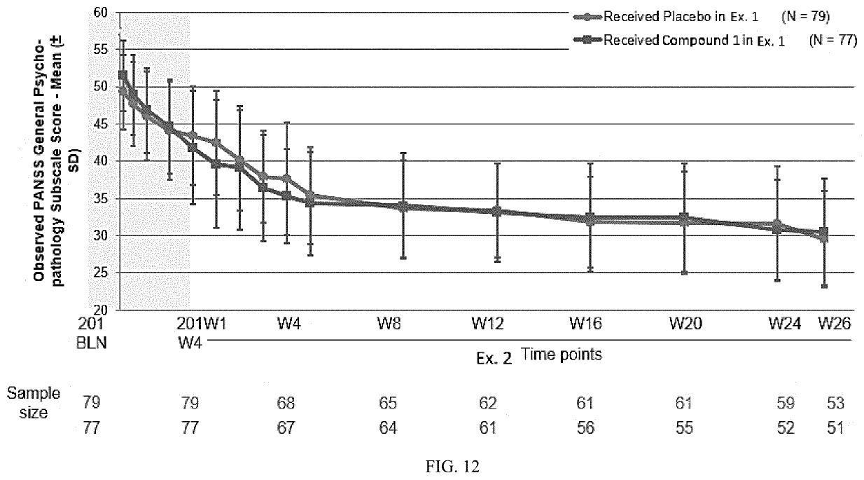

FIG. 12 shows the PANSS general psychopathology subscale score during the extension study with the PANSS general psychopathology subscale score data from the Example 1 study shown for reference.

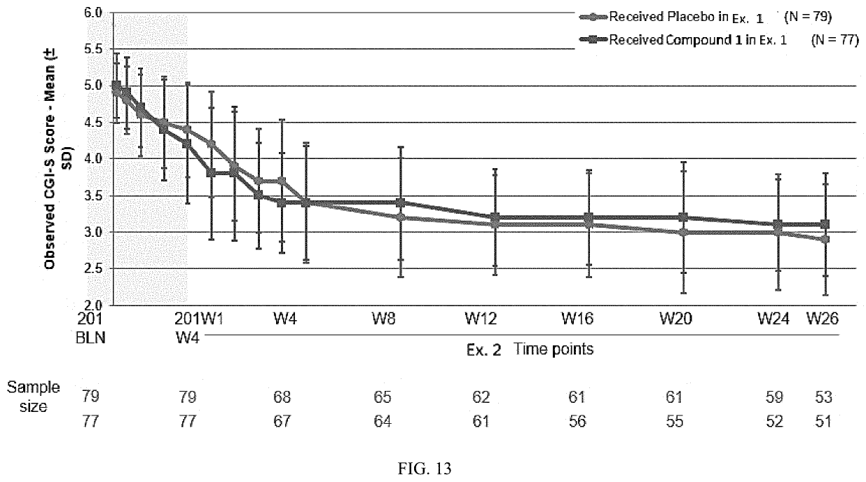

FIG. 13 shows the CGI-S score during the extension study with the CGI-S score data from the Example 1 study shown for reference.

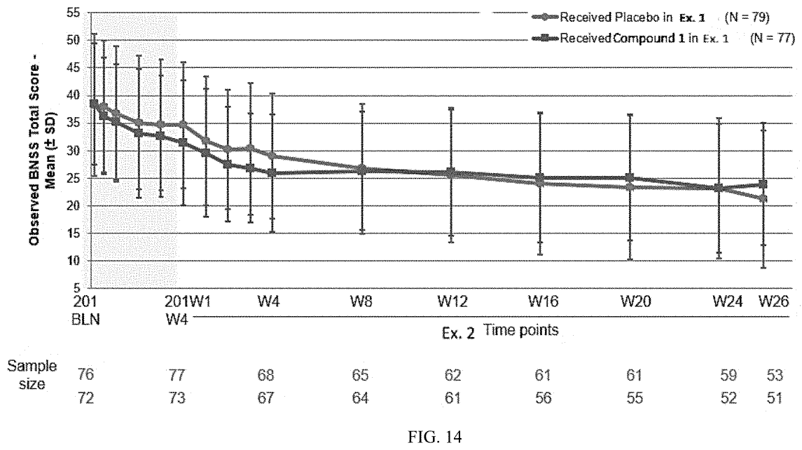

FIG. 14 shows the BNSS total score during the extension study with the BNSS total score data from the Example 1 study shown for reference.

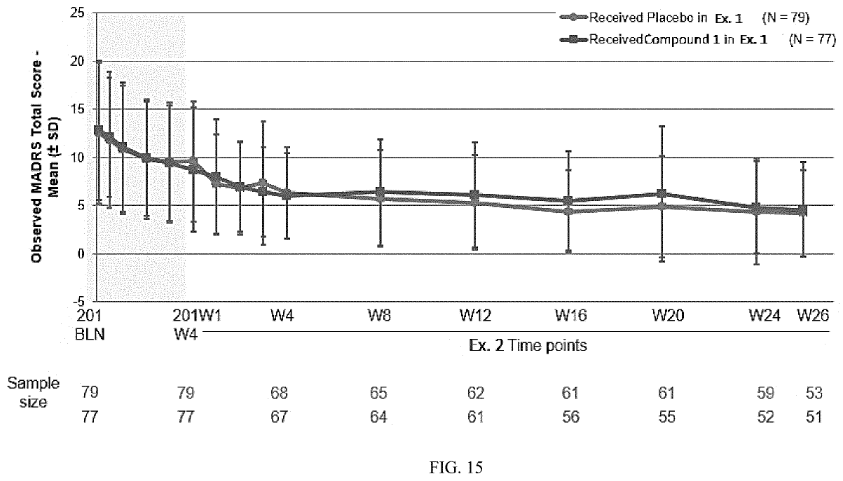

FIG. 15 shows the MADRS total score during the extension study with the MADRS total score data from the Example 1 study shown for reference.

Adverse events were monitored and recorded during the extension study. The incidence of adverse events remained low in both (i) the subjects who previously received placebo and received active treatment for the first time during the extension study, and (ii) the subjects who continued to receive active treatment from the Example 1 study into the extension study. Tables 11-16 show the adverse events experienced during the extension study.

TABLE 11
General Adverse Events

| | Placebo to | Compound 1 to | |
|---|---|---|---|
| | Compound 1 | Compound | Total |
| | (N = 79) | 1 (N = 77) | (N = 156) |
| Preferred Term | n (%) | n (%) | n (%) |
| Subjects with an | 46 (58.2%) | 42 (54.5%) | 88 (56.4%) |
| AE* | | | |
| Schizophrenia | 11 (13.9%) | 8 (10.4%) | 19 (12.2%) |
| Headache | 11 (13.9%) | 7 (9.1%) | 18 (11.5%) |
| Insomnia | 7 (8.9%) | 6 (7.8%) | 13 (8.3%) |
| Anxiety | 4 (5.1%) | 4 (5.2%) | 8 (5.1%) |
| Nasopharyngitis | 3 (3.8%) | 4 (5.2%) | 7 (4.5%) |
| Somnolence | 5 (6.3%) | 2 (2.6%) | 7 (4.5%) |
| Nausea | 4 (5.1%) | 2 (2.6%) | 6 (3.8%) |
| Influenza | 3 (3.8%) | 2 (2.6%) | 5 (3.2%) |
| Irritability | 2 (2.5%) | 3 (3.9%) | 5 (3.2%) |
| Weight decreased | 3 (3.8%) | 2 (2.6%) | 5 (3.2%) |
| Blood prolactin | 1 (1.3%) | 3 (3.9%) | 4 (2.6%) |
| increased | | | |

*Subjects with multiple AEs are counted only once.

TABLE 12
Extrapyramidal Symptoms

| | Placebo to | Compound 1 to | |
|---|---|---|---|
| | Compound 1 | Compound 1 | Total |
| | (N = 79) | (N = 77) | (N = 156) |
| Preferred Term | n (%) | n (%) | n (%) |
| Any subject experiencing | 1 (1.3%) | 4 (5.2%) | 5 (3.2%) |
| an extrapyramidal | | | |
| symptom AE* | | | |
| Parkinsonism | 1 (1.3%) | 1 (1.3%) | 2 (1.3%) |
| Dyskinesia | 0 | 1 (1.3%) | 1 (0.6%) |
| Tremor | 0 | 1 (1.3%) | 1 (0.6%) |
| Restlessness | 0 | 1 (1.3%) | 1 (0.6%) |

*Subjects with multiple EPS AEs are counted only once.

TABLE 13
Prolactin-related Adverse Events

| | Placebo to | Compound 1 to | |
|---|---|---|---|
| | Compound 1 | Compound 1 | Total |
| | (N = 79) | (N = 77) | (N=156) |
| Preferred Term | n (%) | n (%) | n (%) |
| Any subject experiencing | 2 (2.5%) | 4 (5.2%) | 6 (3.8%) |
| a prolactin-related AE* | | | |
| Hyperprolactinemia | 1 (1.3%) | 0 | 1 (0.6%) |
| Blood prolactin increased | 1 (1.3%) | 3 (3.9%) | 4 (2.6%) |
| Menstruation delayed | 0 | 1 (1.3%) | 1 (0.6%) |

*Subjects with multiple prolactin-related AEs are counted only once.

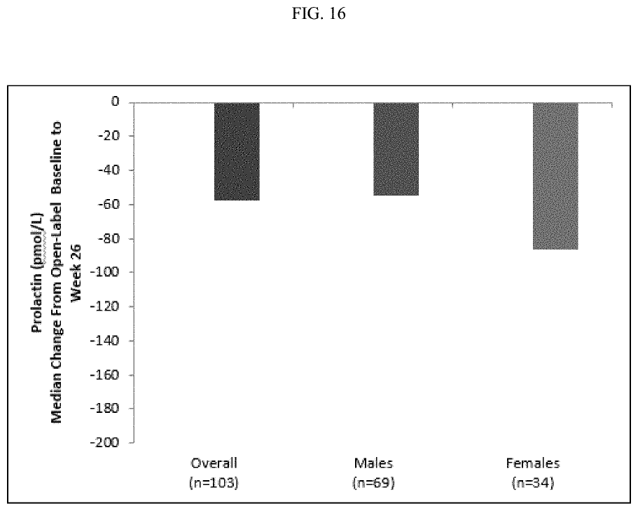

The change in prolactin levels from baseline at week 26 are shown in FIG. 16.

TABLE 14
Cardiovascular Adverse Events

| | Placebo to | Compound 1 to | |
|---|---|---|---|
| | Compound 1 | Compound 1 | Total |
| | (N = 79) | (N = 77) | (N = 156) |
| SOC/Preferred Term | n (%) | n (%) | n (%) |
| Cardiac Disorder | 5 (6.3%) | 2 (2.6%) | 7 (4.5%) |
| Sinus tachycardia | 2 (2.5%) | 1 (1.3%) | 3 (1.9%) |
| Atrioventricular block | 0 | 1 (1.3%) | 1 (0.6%) |
| first degree | | | |
| Angina pectoris | 1 (1.3%) | 0 | 1 (0.6%) |
| Sinus arrhythmia | 1 (1.3%) | 0 | 1 (0.6%) |
| Tachycardia | 1 (1.3%) | 0 | 1 (0.6%) |
| Vascular Disorders | 2 (2.5%) | 0 | 2 (1.3%) |
| Hypotension | 2 (2.5%) | 0 | 2 (1.3%) |
| Investigations | | | |
| Heart rate increased | 1 (1.3%) | 0 | 1 (0.6%) |
| Blood pressure increased | 1 (1.3%) | 0 | 1 (0.6%) |
| Nervous System | | | |
| Disorders | | | |
| Dizziness | 1 (1.3%) | 1 (1.3%) | 2 (1.3%) |

*Subjects with multiple AEs are counted only once.

TABLE 15
Serious Adverse Events

| | Placebo to | | Compound 1 to | | | |
|---|---|---|---|---|---|---|
| | Compound 1 | | Compound 1 | | | |
| | (N = 79) | | (N = 77) | | Total (N = 156) | |
| | | # | | # | | # |
| SOC/Preferred Term | n( %) | Events | n (%) | Events | n (%) | Events |
| Subjects with Any SAE* | 9 (11.4%) | 10 | 6 (7.8%) | 6 | 15 (9.6%) | 16 |
| Psychiatric Disorders | 9 (11.4%) | 10 | 5 (6.5%) | 5 | 14 (9.0%) | 15 |
| Schizophrenia | 7 (8.9%) | 7 | 4 (5.2%) | 4 | 11 (7.1%) | 11 |
| Acute psychosis | 1 (1.3%) | 1 | 0 | 0 | 1 (0.6%) | 1 |
| Depression | 1 (1.3%) | 1 | 0 | 0 | 1 (0.6%) | 1 |
| Psychotic disorder | 0 | 0 | 1 (1.3%) | 1 | 1 (0.6%) | 1 |
| Suicidal ideation | 1 (1.3%) | 1 | 0 | 0 | 1 (0.6%) | 1 |
| Reproductive System | 0 | 0 | 1 (1.3%) | 1 | 1 (0.6%) | 1 |
| and Breast Disorders | | | | | | |
| Uterine Hemorrhage | 0 | 0 | 1 (1.3%) | 1 | 1 (0.6%) | 1 |

*Subjects with multiple SAEs are counted only once

TABLE 16
Adverse Events Leading to Discontinuation

| | Placebo to | | Compound 1 to | | | |
|---|---|---|---|---|---|---|
| | Compound 1 | | Compound 1 | | | |
| | (N = 79) | | (N = 77) | | Total (N = 156) | |
| | | # | | # | | # |
| Preferred Term | n (%) | Events | n (%) | Events | n (%) | Events |
| Any AE leading to | 9 (11.4%) | 9 | 9 (11.7%) | 9 | 18 (11.5%) | 18 |
| discontinuation from | | | | | | |
| study* | | | | | | |
| Schizophrenia | 7 (8.9%) | 7 | 7 (9.1%) | 7 | 14 (9.0%) | 14 |
| Acute Psychosis | 1 (1.3%) | 1 | 0 | 0 | 1 (0.6%) | 1 |
| Anxiety | 0 | 0 | 1 (1.3%) | 1 | 1 (0.6%) | 1 |
| Depression | 1 (1.3%) | 1 | 0 | 0 | 1 (0.6%) | 1 |
| Psychotic disorder | 0 | 0 | 1 (1.3%) | 1 | 1 (0.6%) | 1 |

*Subjects with multiple AEs are counted only once

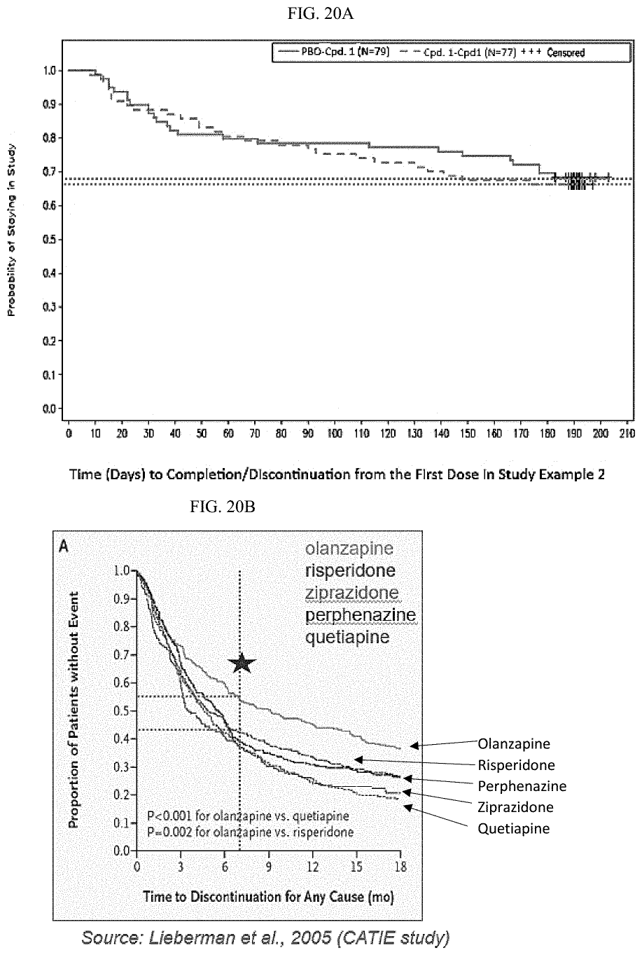

FIG. 20A shows the time to all causes of discontinuation in the extension study. FIG. 20B shows the time to discontinuation for several other drugs: olanzapine, risperidone, ziprazidone, perphenazine, and quetiapine.

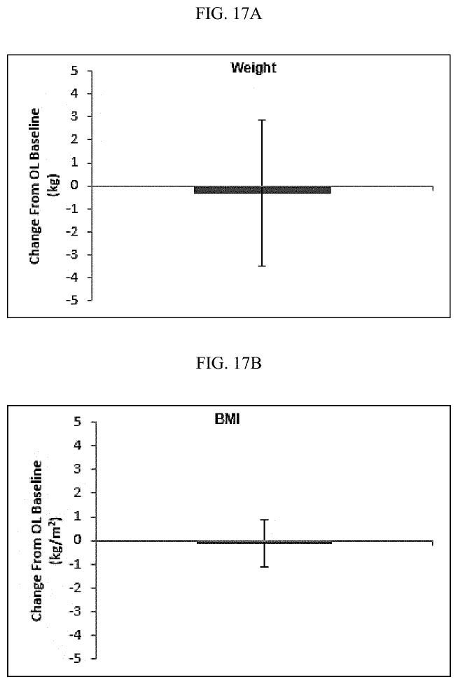

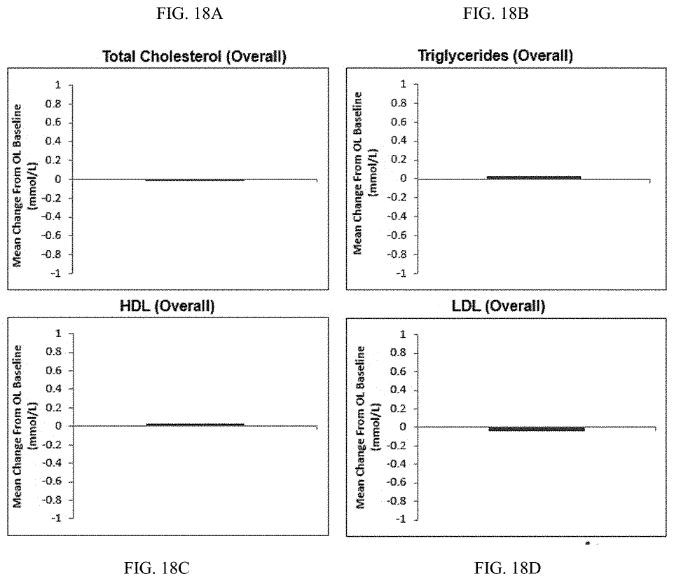

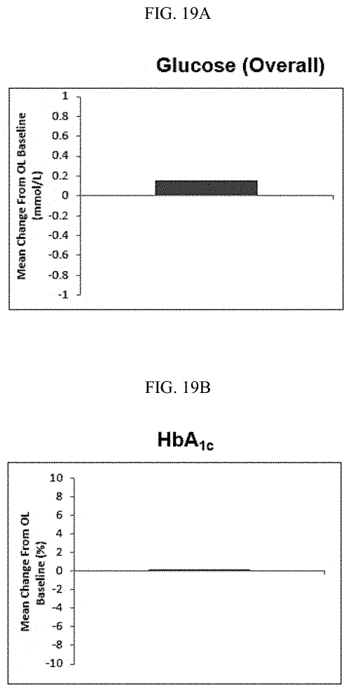

Additional clinical measurements were taken during the study. The change in weight and BMI from open-label baseline (i.e., at start of extension study) at week 26 are shown in FIGS. 17 A and B. The change in lipid measurements (total cholesterol, triglycerides, HDL, LDL) from open-label baseline are shown in FIG. 18 A-D. The change in glycemic measures (glucose, HbA1c) from open-label baseline are shown in FIGS. 19 A and B.

Functional improvement was also measured by UPSA-B score, a performance-based skills assessment. Compound 1 improved UPSA-B total score in the subjects from an average of about 76 to an average of about 84 (effect size of 0.66) during the 26-week period.

Overall, the extension study demonstrated a high completion rate; continued improvement in symptoms of schizophrenia (i.e., improved efficacy scores); very low incidence of EPS-related, prolactin-related, and cardiovascular-related adverse events; and minimal changes in weight, lipids and glycemic measures.

### Example 3: Class-Effect Adverse Events Across Antipsychotics

The antipsychotic class of pharmaceutical compounds is, in part, characterized by certain adverse event risks associated with their use in treating schizophrenia, bipolar and depression patient populations. The Medical Dictionary for Regulatory Activities (MedDRA) is an internationally used set of terms relating to medical conditions, medicines and medical devices, including adverse events. Using MedDRA's standardization of terms (preferred terms), a list of preferred terms of antipsychotic-class related adverse events was established based on reportings to the FDA real-world Adverse Event Reporting Database (FAERS). In particular, FAERS was used to identify preferred terms associated with the 11 most recently FDA-approved antipsychotics (aripiprazole, asenapine, brexpiprazole, cariprazine, iloperidone, lurasidone, olanzapine, paliperidone, quetiapine, risperidone, and ziprasidone). The preferred terms cover a variety of medical systems and organs symptoms. A total of over 9,500 adverse event records were generated using 2018 2nd Quarter data deployed into the Empirica Signal server.

The preferred terms for adverse events of the pool of 11 antipsychotics were ranked by relative risk using a calculated empirical bayes geometric mean (EBGM). Preferred terms that correspond to individual symptoms of schizophrenia and/or bipolar disorders, such as those that correspond with individual items of a psychiatric rating scale used in clinical trials of schizophrenia or bipolar disorder (e.g., PANSS, MADRS) were selected and flagged as disease-related and were not analyzed as side effects of medication. A higher EBGM value for a given drug corresponds with a greater statistical association between the preferred term/adverse event and the drug, compared to all other drugs and all other preferred terms/adverse events. Here, a rank ordering by EBGM values was created to list the preferred terms/adverse events describing the effect of antipsychotics (calculated as an overall pool of 11 antipsychotics) as a class. Accordingly, a compound that causes a clinically significant portion of the treated patient population to have adverse events with preferred terms among the high-ranking (for example, the preferred terms having EGBM values above a threshold) can be considered to have an adverse event profile similar to the class of antipsychotics.

In an example, the preferred terms of association for the pool of 11 antipsychotics are shown in Table 17 below. A compound exhibiting a clinically significant portion of a patient population with adverse events matching these exemplified preferred terms can be considered to have an adverse event profile similar to the class of antipsychotics.

TABLE 17
Selected Preferred Terms of Greatest Association
for the Pool of 11 Antipsychotics

| Adverse Event | Empirical Bayes Geometric Mean |
|---|---|
| (Preferred Term) | (EBGM) |
| Hyperprolactinaemia | 30.7 |
| Blood prolactin abnormal | 24.7 |
| Blood prolactin increased | 20.5 |
| Galactorrhoea | 19.4 |
| Cogwheel rigidity | 17.9 |
| Obesity | 17.9 |
| Metabolic syndrome | 17.7 |
| Akathisia | 15.4 |
| Oromandibular dystonia | 15.3 |
| Parkinsonism | 12.3 |
| Drooling | 10.4 |
| Oculogyric crisis | 9.35 |
| Obsessive-compulsive disorder | 9.17 |
| Muscle rigidity | 8.6 |
| Type 2 diabetes mellitus | 8.44 |
| Diabetes mellitus | 8.39 |
| Overweight | 8.01 |
| Parkinsonian gait | 7.69 |
| Tongue spasm | 7.42 |
| Tardive dyskinesia | 7.24 |
| Bradykinesia | 7.18 |
| Tic | 6.59 |
| Psychomotor retardation | 6.51 |
| Extrapyramidal disorder | 6.42 |
| Enuresis | 6.4 |
| Glucose tolerance impaired | 6.22 |
| Salivary hypersecretion | 6.21 |
| Dystonia | 6.09 |
| Glycosuria | 6.09 |
| Restlessness | 6.02 |
| Torticollis | 6.02 |
| Impaired fasting glucose | 5.88 |
| Dermatillomania | 5.88 |
| Body mass index increased | 5.81 |
| Hyperkinesia | 5.69 |
| Hepatitis viral | 5.64 |
| Dyskinesia | 5.59 |
| Blood triglycerides increased | 5.52 |
| Electrocardiogram QT prolonged | 5.44 |
| Dyssomnia | 5.34 |
| Orthostatic hypertension | 5.29 |
| Bruxism | 5.11 |
| Increased appetite | 5.1 |
| Excessive eye blinking | 5.01 |
| Pancreatitis chronic | 4.94 |
| Weight increased | 4.86 |
| Dyslipidaemia | 4.75 |
| Restless legs syndrome | 4.22 |
| Tongue biting | 4.2 |
| Nuchal rigidity | 4.13 |

The over 9,500 preferred terms for adverse events of the pool of 11 antipsychotics were used to query clinical trial data from Example 1 (4 week study). The ranking, by EBGM, of the preferred terms for Compound 1 is provided in Table 18. Compound 1 demonstrated a clinically insignificant occurrence of adverse events (e.g., hyperprolactinaemia, blood prolactin abnormal, blood prolactin increased, galactorrhoea, cogwheel rigidity, obesity, metabolic syndrome, etc.) associated with the current antipsychotic class, as defined by the preferred terms of greatest relative risk in real-world adverse event reporting databases (e.g., class-related adverse events). Also, preferred terms observed in subjects who were administered placebo as a comparator to Compound 1 showed similar occurrence of adverse events. Accordingly, Compound 1 does not exhibit an adverse event profile matching the antipsychotic class effect.

### Example 4: Pharmacokinetics

The pharmacokinetics (PK), safety, and tolerability of Compound 1 were evaluated in single ascending doses (5 mg to 125 mg and 25 mg to 150 mg) in healthy adult male subjects and in adult male and female patients with schizophrenia, respectively, or as multiple ascending doses (10, 25, 50, 75, and 100 mg, once daily) in adult male and female patients with schizophrenia. Blood samples from time 0 to 144 hours post-dose were collected for PK analysis. Safety evaluations included adverse events, vital signs, clinical laboratory tests, physical and neurological examinations, C-SSRS, 12-lead ECGs, and safety EEGs.

Healthy Adult Male Subjects, Single Ascending Doses

The safety, tolerability and maximum tolerated dose (MTD) of a single oral dose of Compound 1 was tested in 39 normal healthy adult male subjects. To be included, the subject had to be a healthy male between the ages of 18-50 (inclusive), have a BMI between 16-32 kg/m2 (inclusive), have no diagnosis of schizophrenia, and not be using CNS active drugs or CYP2D6 inhibitors concomitantly.

Single doses of Compound 1, at concentrations of 5 mg, 10 mg 25 mg 50 mg, 100 mg and 125 mg were given to the subjects. Six subjects were present in each group, except for the 125 mg group, where nine subjects were administered the dose, and there were 13 placebo subjects. There were no deaths, nor were there clinically significant treatment-emergent changes in laboratory parameters in this study. The results of the plasma PK parameters are shown below in Table 19:

TABLE 19
Plasma PK Parameters Following a Single Oral Dose of Compound 1 In Healthy Adult Male Subjects

| | 5 mg | | 10 mg | | 25 mg | | 50 mg | | 100 mg | | 125 mg | |
|---|---|---|---|---|---|---|---|---|---|---|---|---|
| Parameter | (N = 6) | | (N = 6) | | (N = 6) | | (N = 6) | | (N = 5) | | (N = 9) | |
| Cₘₐₓ, ng/mL (CV %) | 17.3 | (3.7) | 25.7 | (5.7) | 65.4 | (20.4) | 139 | (22.7) | 287 | (58.2) | 379 | (62.3) |
| AUC₀₋ₗₐₛₜ, h · g/mL | 104 | (27) | 223 | (31) | 643 | | 1116 | (15) | 2700 | (63) | 4188 | (32) |
| (CV %) | | | | | | | | | | | | |
| tₘₐₓ, median, h | 1.25 | | 1.75 | | 2.51 | | 2.50 | | 3.00 | | 3.00 | |
| t_{1/2} median, h | 6.8 | (53.0) | 5.7 | (30.8) | 11.3 | (63.9) | 12.0 | (60.3) | 12.2 | (61.6) | 13.6 | (52.5) |
| V_{z}/F, L (CV %) | 489 | (64) | 376 | (64) | 657 | (48) | 763 | (65) | 6450 | (223) | 599 | (41) |
| CL/F, L/h (CV %) | 49.9 | (22.7) | 47.4 | (22.5) | 49.2 | (66.3) | 45.7 | (16.5) | 969 | (236) | 34.4 | (49.8) |

Adult Male and Female Subjects with Schizophrenia, Single Ascending Doses

A study was performed to evaluate the safety, tolerability and MTD of a single oral dose of Compound 1 in male and female subjects with schizophrenia. To be included, the subjects had to be a male or female between the ages of 18-55 (inclusive) and have a BMI between 19.5 kg/m2 and 37 kg/m2 (inclusive). Additionally, the subjects had to meet Diagnostic and Statistical Manual of Mental Disorders Fourth Edition; Text Revision (DSM-IV-TR) criteria for a primary diagnosis of schizophrenia, and not be using CNS active drugs or CYP2D6 inhibitors concomitantly.

Single doses of Compound 1 at concentrations of 25 mg, 50 mg, 100 mg, and 150 mg were given to the subjects. Nine subjects were present in each group, and there were twelve placebo subjects. There were no deaths, nor were there clinically significant treatment-emergent changes in laboratory parameters in this study. The results of the plasma PK parameters are shown below in Table 20:

TABLE 20
Study 2: Plasma PK Parameters Following Single Oral Dose of Compound 1

| | 25 mg | | 50 mg | | 100 mg | | 150 mg | |
|---|---|---|---|---|---|---|---|---|
| Parameter | (N = 9) | | (N = 9) | | (N = 9) | | (N = 9) | |
| Cₘₐₓ, ng/mL (CV %) | 80.0 | (18.6) | 208 | (80.6) | 366 | (96.8) | 450 | (152) |
| AUC_{0-∞}, h · ng/mL (CV %) | 694 | (43.1) | 1791 | (16.4) | 3644 | (20.9) | 50.86 | (43.2) |
| tₘₐₓ, median, h | 1.0 | | 1.5 | | 1.5 | | 4.0 | |
| t_{,1/2}, h (CV %) | 14.4 | (8.0) | 12.4 | (5.6) | 17.1 | (16) | 17.5 | (7.3) |
| V_{z}/F, l/h (CV %) | 750 | (328) | 491 | (207) | 685 | (217) | 789 | (343) |

Study Design: Adult Male and Female Subjects with Schizophrenia, Multiple Ascending Doses: Two-Part Clinical Study: Multiple Dose and 28-Day Open Label

This study was conducted in two parts: a multiple dose study and a 28-day open label study. Compound 1 was evaluated in human adult male and female subjects with a diagnosis of schizophrenia to study its safety, tolerability, and pharmacokinetics in the treatment of schizophrenia. The study had two separate parts, enrolling separate cohorts of patients, but utilizing the same study entry criteria. Part A was a multicenter, randomized, single-blind, placebo-controlled, ascending multiple oral dose study, while Part B was a single site, non-randomized, open-label, study that evaluated the safety, tolerability, and pharmacokinetics of 28 days of treatment with a 75 mg/day dose of Compound 1. Efficacy assessments were performed during open-label treatment in Part B.

Study Entry Criteria:

Male and female subjects, 18 to 55 years old (inclusive), were eligible for enrollment if they met Diagnostic and Statistical Manual of Mental Disorders Fourth Edition; Text Revision (DSM-IV-TR) criteria for a primary diagnosis of schizophrenia. Subjects had to have a body mass index (BMI) of 19.5-37 kg/m² (inclusive); be clinically stable for the previous 6 months; and have a CGI-S score≤4; had a PANSS total score≤80 (with a score≤4 [moderate-or-less] on the following PANSS items: hostility [P7], uncooperativeness [G8]). The subjects were required to remain drug-free during the study period, including no use of antipsychotic medication, antidepressants or mood stabilizers, or prescription or over-the-counter medication including vitamins and dietary supplements. Permitted medications included OTC analgesics, e.g., acetaminophen, hydrocortisone cream, female contraceptives, and medications for stable conditions (e.g., hypertension or hypercholesterolemia), and limited use of lorazepam and zolpidem were allowed during the washout and treatment period.

Study Design: Multiple Dose (Part A):

Sixty subjects were randomized in five ascending dosage cohorts (N=12) and assigned to the following Compound 1 dosage groups: 10 mg, 25 mg, 50 mg, 75 mg, 100 mg (administered orally in a single daily dose while fasting). In each cohort, subjects were randomized in a 3:1 ratio to receive either Compound 1 (N=9) or matching placebo (N=3) for seven days.

Of the 60 subjects randomized, 71.7% were male, the mean age was 41.8 (range, 24-55), 85.0% were African-American, and the mean PANSS total score was 59.4. All but one subject completed the study per protocol. This subject discontinued study due to an SAE of psychotic disorder (judged not to be related to study drug).

Table 21 shows the pharmacokinetic parameters following (A) a single oral dose of the ascending concentrations of Compound 1 on Day 1 and (B) multiple doses of Compound 1 on Day 7:

TABLE 21
Pharmacokinetic Parameters Following (A) Single
Oral Dose, or (B) Multiple Doses of Compound 1

| | 10 mg | 25 mg | 50 mg | 75 mg | 100 mg |
|---|---|---|---|---|---|
| Parameter | (N = 9) | (N = 9) | (N = 9) | (N = 9) | (N = 9) |
| | A. Single Dose (Day 1) | | | | |
| Cₘₐₓ, ng/mL (CV %) | 27.0 (38.1) | 95.2 (31.1) | 198 (35.1) | 281 (24.9) | 375 (27.8) |
| tₘₐₓ, median, h | 2.0 | 2.0 | 4.0 | 4.0 | 4.0 |
| AUC₀₋₂₄, h · ng/mL (CV %) | 196 | 928 | 1620 | 2296 | 3781 |
| | B. Multiple Doses (Day 7) | | | | |
| Cₘₐₓ, mean, ng/mL (CV %) | 28.3 (38.3) | 112 (26.6) | 203 (16.8) | 246 (38.2) | 431 (12.2) |
| tₘₐₓ, median, h | 2.0 | 2.0 | 4.0 | 2.0 | 2.0 |
| t_{,1/2}, h | 21.5 | 21.0 | 20.2 | 21.3 | 21.1 |
| AUC₀₋₂₄, h · ng/mL | 217 | 1158 | 2039 | 2553 | 4718 |
| V_{SS}/F, l/h | 2474 | 725 | 716 | 1070 | 650 |
| CL_{SS}/F, l/h | 97.7 | 23.8 | 24.7 | 40.5 | 21.9 |

Means shown, except for tₘₐₓ where the median is reported
Cₘₐₓ, maximum plasma concentration;
CV %, percent coefficient of variation;
tmax, time to Cmax, t_{1/2}, elimination half-life;
AUC₀₋₂₄, area under the plasma concentration time curve from 0-24 hours post-dose;
Vss/F, apparent volume of distribution at steady state;
Cl_{SS}/F, clearance at steady state

In the dose range of 10-100 mg/day, Compound 1 was found to be dose proportional for Cₘₐₓ at Day 7 (β=1.17 [95% CI: 0.98-1.37]), and approximately dose proportional for AUC₀₋₂₄ (β=1.30 [95% CI: 1.10-1.50]). The mean Vₛₛ/F and mean CLₛₛ/F of Compound 1 at Day 7 did not appear to change substantially with increases in dose.

Study Design: Open-Label Dosing for 28 Days (Part B):

In the open-label study, adult patients (N=16) diagnosed with schizophrenia were admitted to the clinic and completed a washout of their prior antipsychotic medications. After successful washout, subjects were dosed with Compound 1 (75 mg/day) for 28 days. Patients remained in the clinic for the first two weeks of dosing and outpatient for the remaining two weeks of dosing. Safety assessments included incidence of adverse events, clinical laboratory measures, and movement disorder scales (BARS, AIMS and M-SAS). The effect of Compound 1 on the positive and negative syndrome (PANSS) scale and clinical global impression-severity (CGI-S) was also assessed.

A total of 14 subjects completed the 28-day open-label study. Two subjects discontinued the study after two weeks due to multiple mild adverse events. Of the 16 subjects randomized, 50% were male, the mean age was 31.8 (range, 23-40), 75.0% were African-American, and the mean PANSS total score was 73.3.

No exacerbations of schizophrenia symptoms were observed in any subject. There were no clinically significant treatment-emergent changes in laboratory parameters; ECG parameters, including QTcB and QTcF intervals; neurological examinations; or movement disorder effects as measured by the Barnes Akathisia Scale, Abnormal Involuntary Movement Scale, or the modified Simpson-Angus Scale in either Part A or Part B of the study, nor were there any deaths.

The pharmacokinetic parameters following multiple 75 mg/day doses of Compound 1 (Part B, Day 13) were as follows: Cₘₐₓ (CV %), 316 ng/mL (17.5%); tₘₐₓ (median), 4.0 hours; AUC₀₋₂₄, 3487 h-ng/mL. Visual inspection of mean trough plasma concentrations of Compound 1 showed that steady state was achieved by Day 7.

In addition, treatment with Compound 1 demonstrated improvement in efficacy measures (PANSS total score, CGI-S) compared with baseline. Furthermore, ad hoc subgroup analyses showed a significantly greater decrease from baseline in PANSS total scores at the end of the 28-day treatment period in subjects who had less frequent hospitalizations per year of illness compared with subjects who had more frequent hospitalizations per year of illness.

In summary, no safety issues were observed with multiple oral doses of Compound 1 in doses ranging from 10-100 mg/d for seven days, or in a dose of 75 mg/d for 28 days. There were no clinically significant, treatment-emergent changes in vital signs, physical examination, laboratory parameters, or ECG parameters, including QTcF intervals. No subject had treatment-emergent suicidal ideation or behavior. Treatment with Compound 1 at 75 mg/d for 28 days was associated with improvement in PANSS total score that was greater in patients with high baseline PANSS total scores, lower age, and fewer hospitalizations. Results from this study demonstrate an acceptable safety and tolerability profile of Compound 1 (75 mg/day) up to 28 days in patients with schizophrenia.

### Example 5: Preparation of (S)-4,5-dihydro-7H-thieno[2,3-c]pyran-7-yl)-N-methylmethanamine (“(S)-TPMA”) HCl of Crystalline Form A (i.e., Crystalline Form A of the HCl Salt of Compound 1)

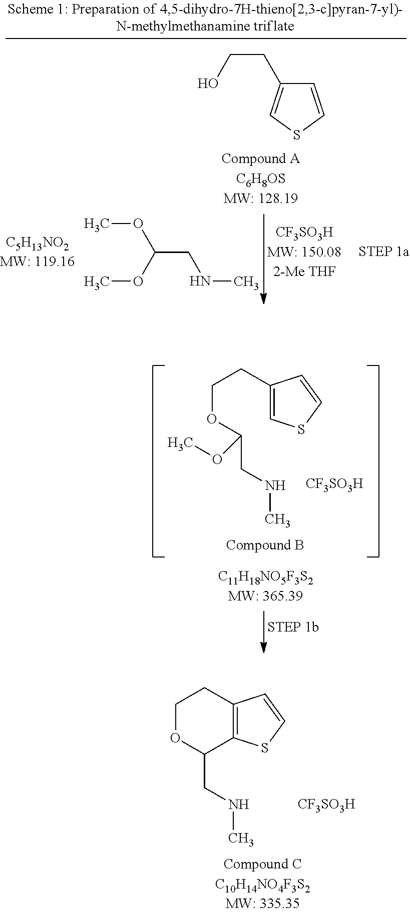

77 g of 3-thiopheneethanol (Compound A) was added to a solution of 69 g of N-methylaminoacetaldehyde dimethyl acetal in 595 ml (508 g) of 2-methyl tetrahydrofuran (THF). After stirring for 5 minutes 99 g (58.2 ml) trifluoromethanesulfonic acid was added. It is important to note that trifluoromethanesulfonic acid is a very hazardous substance. The reaction was heated to reflux for 1 hour (80±2° C.). The reaction was then distilled at atmospheric pressure to remove the byproduct methanol and to reduce the reaction volume to a targeted volume of 460 ml over 4-8 hours. The reaction was judged complete when 1.0% or less (HPLC Peak Area % of peaks of interest, Compounds A, B and C) of compound 1B remained by a sample HPLC analysis.

If the amount of Compound B was greater than or equal to 1%, an appropriate amount of 2-methyl THF was added and distillation continued to the target volume. If the target volume was reached before the completion of reaction (about 4 hours), 300 ml 2-methyl THF was added to the reaction for continuation of the distillation. After reaction completion, the reaction was cooled to about 40-50° C. and concentrated to a target volume of 325 ml under vacuum distillation. 218 g (325 ml) of Toluene was then added over about 15 minutes and the reaction slurry formed was then stirred for 1 hour at 50±2° C., and then cooled to 20±2° C. linearly over 1 hour 45 minutes while being stirred. The slurry was filtered and the product cake was washed with a 2-methyl THF and toluene mixture (1:1 volume/volume). The wet-cake was dried under vacuum at 40±5° C. to constant weight to yield racemic TPMA triflate (Compound C) as an off-white solid and a yield of about 79% was obtained.

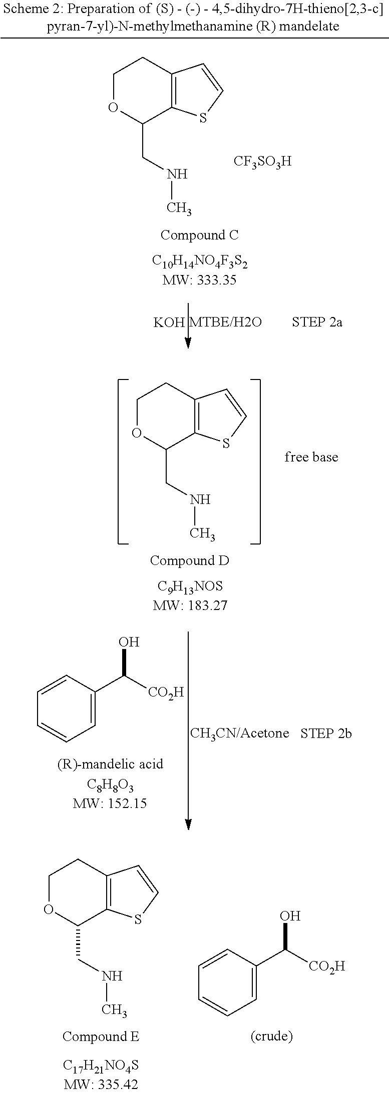

To a suspension of 555.3 g of TPMA triflate (Compound 1C) in 1668 ml methyl tert-butyl ether (MTBE) was added 1076 g of 1.77 N aqueous KOH. After stirring for 10 minutes the pH was checked and if less than 13 small portions of 1.77 N KOH were added until the pH was 13 or greater. The aqueous and organic layers were allowed to settle and separate and separately collected. The MTBE (upper) organic phase layer was held for further processing. The aqueous (bottom) phase layer was extracted twice with MTBE (first with 835 ml and second with 150 ml), the organic (MBTE) layer being collected each time. The MTBE layers (organic layers) were combined, and washed with 20% aqueous NaCl solution (492.9 g) stirred and the phases allowed to settle for 10 minutes. The aqueous layer was removed and the remaining MTBE organic layer was distilled at atmospheric pressure to reduce the reaction volume to a targeted level of 1.9 L. After completion, the process stream was cooled to about 45° C. and concentrated to a target volume of 890 ml under vacuum distillation while maintaining the temperature at 35-45° C. The water content after vacuum distillation was found to be about 0.37% buy weight. A filtration was then performed to remove insoluble materials using a wash of 204 ml MTBE, and the process stream (filtrate) was transferred to a clean reactor. 2512 mL of acetonitrile was added and a solvent switch was performed via vacuum distillation at 35-45° C. to the targeted volume of 800 ml, and the reactor washed with 150 ml of acetonitrile and added to the process stream. The resulting process stream, acetonitrile solution of TPMA free base (Compound D). Acetonitrile was then added, if needed, to the acetonitrile solution of TPMA free base (Compound D) to achieve about a 33 weight % of Compound D.

A solution of 250.3 g of (R)-mandelic acid in 1828 ml of acetone was warmed to 48±2° C. The TPMA free base solution in acetonitrile (917.7 g solution of 302.1 g of Compound D in acetonitrile) was then added at a rate maintaining the reaction temperature below 51° C. After stirring at 48±2° C. for about 10 minutes the process stream was cooled to 45±2° C. and charged with 1.5 g of (S)-TPMA (R)-mandelate seed crystals. The process stream was stirred at 45±2° C. for about 30 minutes and cooled linearly to 21±2° C. over 90 minutes. After holding at 45±2° C. for about 30 minutes the process stream was cooled linearly to 10±2° C. over 45 minutes. The reaction slurry was then stirred for 60 minutes at 10±2° C., filtered and the product cake was washed with acetone/CH₃CN mixture (2.3:1 weight/weight). The wet-cake was dried under vacuum at 40±2° C. to a constant weight to yield crude (S)-TPMA (R)-mandelate (Compound E) as a white crystalline solid, and a yield of about 41% was obtained.

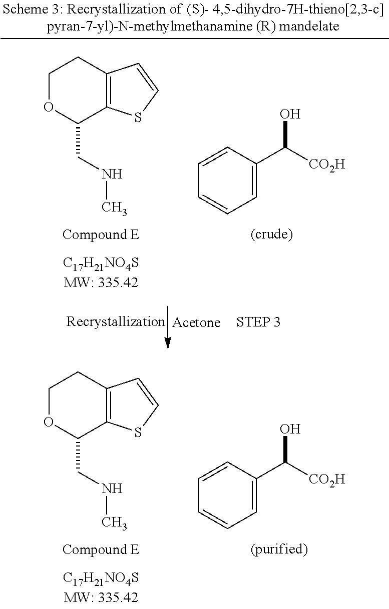

A slurry of crude (S)-TPMA (R)-mandelate (Compound E) from Scheme 2 (200.1 g) in 4205 ml of acetone was warmed to about 56° C. (boiling point of acetone) and stirred until a clear solution was obtained. After cooling the solution to 47±2° C. over approximately 20 minutes (S)-TPMA (R)-mandelate seed crystals were added. The process stream was stirred at 47±2° C. for about 30 minutes and cooled linearly to 21±2° C. over 90 minutes. After holding at 21±2° C. for about 30 minutes the slurry was cooled linearly to 10±2° C. over 45 minutes and then stirred for 1 hour at 10±2° C., filtered, and the product cake was washed with acetone (twice with 401 ml each time). The wet-cake was dried under vacuum at about 40±2° C. to a constant weight to yield (S)-TPMA (R)-mandelate (purified Compound E) as a white crystalline solid, and a yield of about 77% was obtained.

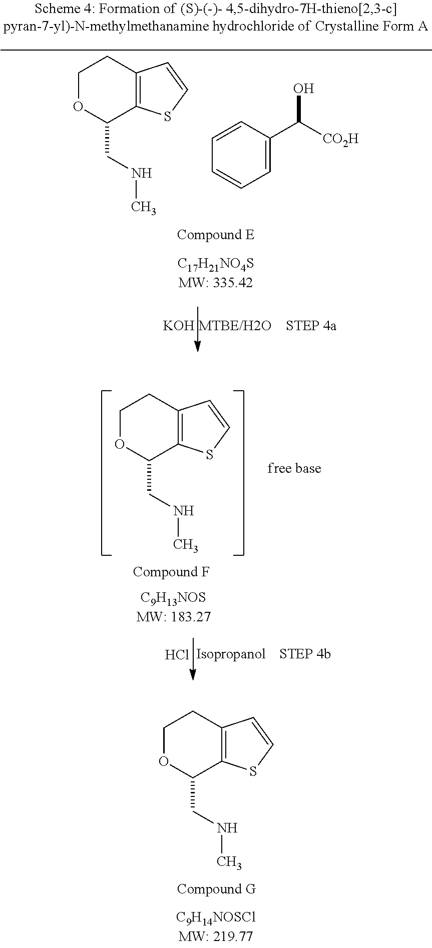

Scheme 4 of the present example provides a reactive crystallization of (S)-(−)-4,5-dihydro-7H-thieno[2,3-c]pyran-7-yl)-N-methylmethanamine HCl, ((S)-TPMA HCl), as crystalline Form A. As (S)-TPMA HCl crystallizes it displays two distinct morphologies (polymorphs), the first a block like crystal (Form A) and the second a needle like crystal (Form B). Based on single crystal x-ray diffraction studies, described herein, Form A has a monoclinic crystal system while Form B has an orthorhombic crystal system.

To a suspension of (S)-(4,5-dihydro-7H-thieno[2,3-c]pyran-7-yl)-N-methylmethanamine (R)-mandelate salt (Compound E) from Scheme 3 (100 g) in 305 ml of MTBE, 172.5 ml of a 10% KOH aqueous solution was added. After stirring for 10 minutes at 20±2° C. the aqueous and organic layers were separated. The organic MTBE (upper) layer was saved for further processing. If the pH of the aqueous layer was less than 13, small portions of the 19% KOH solution were added to raise the pH to 13. The aqueous (bottom) layer was back extracted twice with MTBE (first with 208 ml MTBE, second with 155 ml MTBE), the organic layer being saved for further processing each time. The saved organic layers were combined, and the combined organic layer was subjected to azeotropic distillation to remove water and distilled at atmospheric pressure to a target volume of 140 ml. The process stream was then filtered, to remove insoluble material (e.g. salt precipitated due to removal of water), and the filtrate transferred to a clean reactor. 775 ml of Isopropanol was added (resulting in a total process stream volume of about 1030 ml) and a solvent switch was performed via vacuum distillation at less than 45° C. to provide a 10%-15% solution of (S)-(4,5-dihydro-7H-thieno[2,3-c]pyran-7-yl)-N-methylmethanamine in isopropanol.

In various embodiments, the amount of isopropanol added was selected so to adjust the freebase (Compound F) weight % concentration to 6-7%. The reaction mixture was cooled to 20±2° C., filtered, the filter washed with 78 ml isopropanol, and the filtrate transferred to a clean reactor. 201.6 g of a 6% HCl (w/w) solution in isopropanol was then added into the reactor over 45 minutes at about 20±2° C. It is to be understood that in various embodiments, the target amount of HCl is about 10% excess relative to the freebase (Compound F) molar equivalence. The HCl was added as follows, the first 10% was added over 15 mins, the next 30% was added over 15 mins, and the remainder was then added over 15 mins. A retreat curve impeller at 160 rpm to 270 rpm in a 5 L scale reactor was used, with a process stream volume of about 740 ml, and produced reasonable-sized particles and particle distributions with no obvious agglomeration observed. The slurry formed was warmed up to 40±2° C. linearly over 20 minutes and held at 40±2° C. for about 30 minutes. It was then cooled linearly to 20±2° C. over 20 minutes. After stirring at 20±2° C. for about 30 minutes the slurry was filtered and the product cake was washed with isopropanol (first with 86 ml, second with 92 ml). The cake was dried under vacuum at 40±2° C. to a constant weight to yield (S)-(−)-TPMA hydrochloride (Compound G) as a white crystalline solid, and a yield of about 84% was obtained.

In Step 4b of Scheme 4, slow addition, that is here, low supersaturation generation rate, favors the formation of desired block (S)-(−)-TPMA HCl crystals (Form A) while decreasing the generation the undesired needles (Form B) and higher temperature favored the formation of the block like Form A crystals over Form B.

An ¹H NMR spectrum of the (S)-(−)-(4,5-dihydro-7H-thieno[2,3-c]pyran-7-yl)-N-methylmethanamine hydrochloride (Compound G) obtained in this Example 2 has the following characteristics: ¹H NMR (300 MHz, DMSO-d₆); δ (ppm): 2.53 (s, 3H, —CH₃); 2.5-2.8 (m, 2H, —CH₂—); 3.15-3.37 (2dd, 2H, CH₂—NH); 3.77 and 4.13 (2ddd, 2H, CH₂—O); 5.19 (dd, 1H, O—CH—C═); 6.95 (d, J=5 Hz, 1H, HC═); 7.49 (dd, J=5 Hz, 1H, HC═); 9.12 (br, 2H, NH₂⁺).

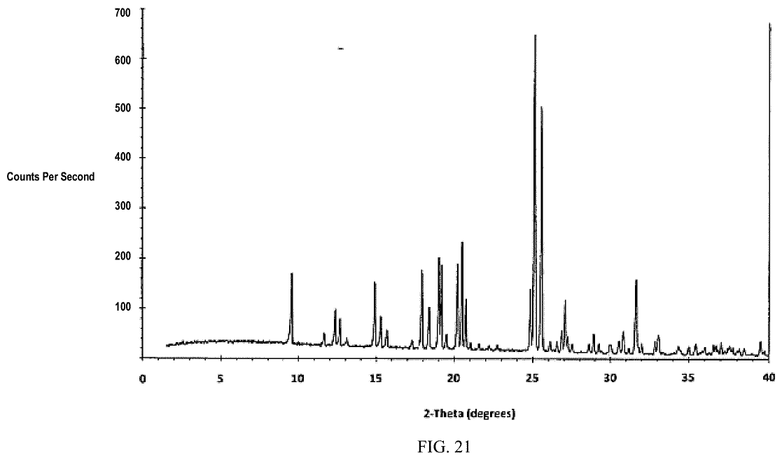

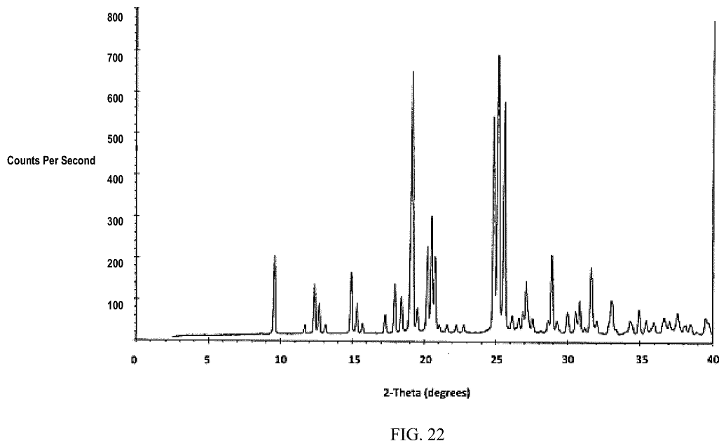

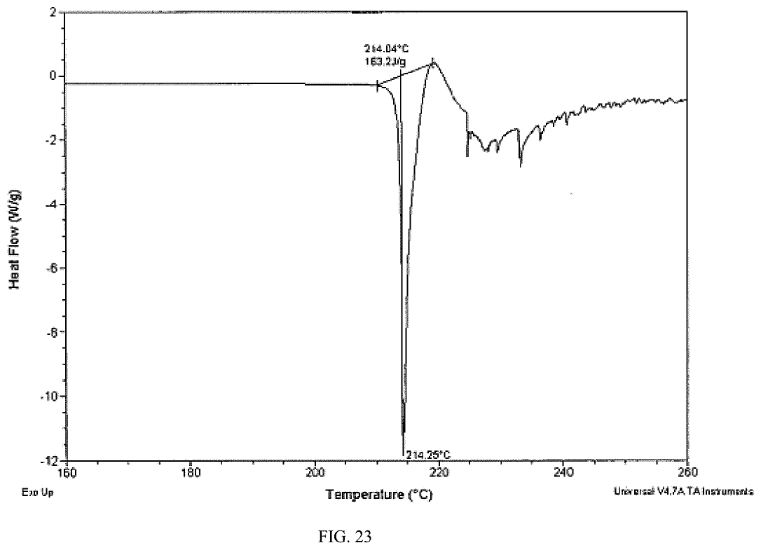

FIGS. 21 and 22 present XRPD patterns for (S)-(4,5-dihydro-7H-thieno[2,3-c]pyran-7-yl)-N-methylmethanamine hydrochloride of Form A; FIG. 21 is the XRPD measured in transmission mode and FIG. 22 in reflection mode. FIG. 23 is a DSC thermogram for (S)-(4,5-dihydro-7H-thieno[2,3-c]pyran-7-yl)-N-methylmethanamine hydrochloride, of polymorph Form A.

Various preferred embodiments [A] to [CB] of the invention can be described in the text below:

### Embodiment A

A method of treating a neurological or psychiatric disease or disorder, in a patient in need thereof, without causing a clinically significant risk of adverse events, comprising administering to the patient a therapeutically effective amount of Compound 1

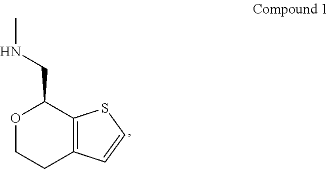

or a pharmaceutically acceptable salt thereof, wherein the patient does not experience a clinically significant adverse event.

### Embodiment B

A method of treating a neurological or psychiatric disease or disorder, in a patient in need thereof, without causing a clinically significant risk of adverse events, comprising administering to the patient a therapeutically effective amount of Compound 1

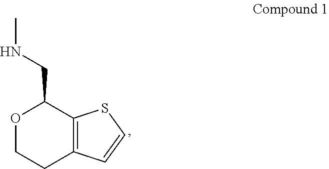

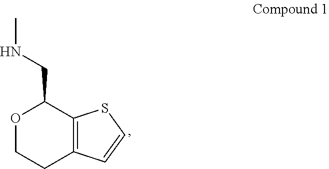

or a pharmaceutically acceptable salt thereof.

### Embodiment D

A method of treating schizophrenia, in a patient in need thereof, without causing a clinically significant risk of adverse events, comprising administering to the patient a therapeutically effective amount of Compound 1

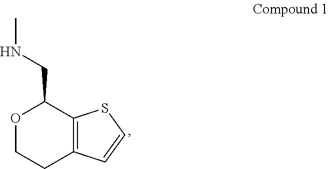

or a pharmaceutically acceptable salt thereof.

### Embodiment E

A method of treating a patient having schizophrenia without causing a clinically significant risk of adverse events, comprising administering to the patient a therapeutically effective amount of Compound 1

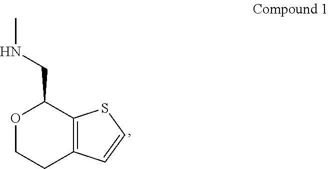

or a pharmaceutically acceptable salt thereof.

### Embodiment F

A method of treating a neurological or psychiatric disease or disorder in a patient, comprising administering to the patient a therapeutically effective amount of Compound 1

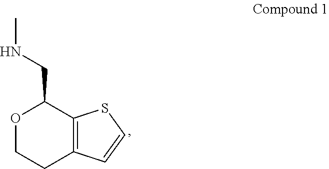

or a pharmaceutically acceptable salt thereof, wherein the method minimizes adverse events associated with antipsychotic agents with affinity to dopamine D2 in the patient.

### Embodiment G

A method of treating a neurological or psychiatric disease or disorder in a patient, comprising administering to the patient a therapeutically effective amount of an antipsychotic agent with no direct affinity to dopamine D2 receptors, wherein the method is substantially devoid of adverse events in the patient, wherein the adverse events are associated with antipsychotic agents with affinity to dopamine D2.

### Embodiment H

A method of Embodiment [G] above, or according to other embodiments of the invention, wherein the antipsychotic agent with no direct affinity to dopamine D2 receptors is Compound 1

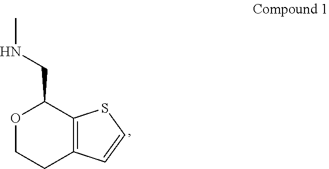

or a pharmaceutically acceptable salt thereof.

### Embodiment I

A method of minimizing adverse events in a patient in need of treatment for a neurological or psychiatric disease or disorder, the method comprising administering to the patient a therapeutically effective amount of an antipsychotic agent with no direct affinity to dopamine D2 receptors, wherein the antipsychotic agent is Compound 1

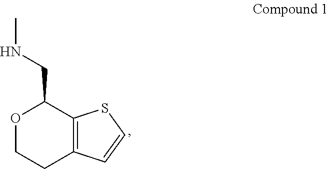

or a pharmaceutically acceptable salt thereof, and wherein the method minimizes adverse events associated with antipsychotic agents with affinity to dopamine D2 receptors.

### Embodiment J

A method of any one of Embodiments [A] to [I] above, or according to other embodiments of the invention, wherein the neurological or psychiatric disease or disorder is schizophrenia.

### Embodiment K

A method of Embodiment [J] above, or according to other embodiments of the invention, further comprising treating negative symptoms of schizophrenia.

### Embodiment L

A method of any one of Embodiments [A] to [I] above, or according to other embodiments of the invention, wherein the neurological or psychiatric disease or disorder is schizophrenia spectrum disorder, schizophrenia negative symptoms, attenuated psychosis syndrome, prodromal schizophrenia, delusional disorder, psychosis, psychotic disorder, delirium, Tourette's syndrome, post-traumatic stress disorder, behavior disorder, affective disorder, depression, bipolar disorder, major depressive disorder, dysthymia, manic disorder, seasonal affective disorder, obsessive-compulsive disorder, narcolepsy, REM behavior disorder, substance abuse or dependency, Lesch-Nyhan disease, Wilson's disease, autism, Alzheimer's disease agitation and psychosis, or Huntington's chorea.

### Embodiment M

A method of any one of Embodiments [A] to [J] or [L] above, or according to other embodiments of the invention, wherein said neurological or psychiatric disease or disorder is selected from schizophrenia, attenuated psychosis syndrome, prodromal schizophrenia, schizoid personality disorder, and schizotypal personality disorder.

### Embodiment N

A method of Embodiment [L] above, or according to other embodiments of the invention, wherein said psychosis is selected from organic psychosis, drug-induced psychosis, Parkinson's disease psychosis, and excitative psychosis.

### Embodiment O

A method of any one of Embodiments [A] to [N] above, or according to other embodiments of the invention, wherein the patient fails to respond adequately to antipsychotic agents which are at least one typical antipsychotic agent or at least one atypical antipsychotic agent.

### Embodiment P

A method of any one of Embodiments [A] to [O] above, or according to other embodiments of the invention, wherein Compound 1, or a pharmaceutically acceptable salt thereof, comprises an HCl salt of Compound 1.

### Embodiment Q

A method of any one of Embodiments [A] to [P] above, or according to other embodiments of the invention, wherein Compound 1, or a pharmaceutically acceptable salt thereof, is administered orally.

### Embodiment R

A method of any one of Embodiments [A] to [Q] above, or according to other embodiments of the invention, wherein Compound 1, or a pharmaceutically acceptable salt thereof, is administered daily.

### Embodiment S

A method of any one of Embodiments [A] to [R] above, or according to other embodiments of the invention, wherein Compound 1, or a pharmaceutically acceptable salt thereof, is administered at about 50 mg or about 75 mg per day.

### Embodiment T

A method of any one of Embodiments [A] to [S] above, or according to other embodiments of the invention, wherein Compound 1, or a pharmaceutically acceptable salt thereof, is administered daily during a 29-day treatment period.

### Embodiment U

A method of any one of Embodiments [A] to [S] above, or according to other embodiments of the invention, wherein Compound 1, or a pharmaceutically acceptable salt thereof, is administered daily during a 26-week treatment period.

### Embodiment V

A method of any one of Embodiments [A] to [U] above, or according to other embodiments of the invention, wherein a risk of adverse events in the patient is about the same as or similar to placebo.

### Embodiment W

A method of any one of Embodiments [A] to [V] above, or according to other embodiments of the invention, wherein the method minimizes cardiovascular adverse events.

### Embodiment X

A method of any one of Embodiments [A] to [W] above, or according to other embodiments of the invention, wherein the method results in a cardiovascular event in less than or equal to 5% of patients.

### Embodiment Y

A method of any one of Embodiments [A] to [W] above, or according to other embodiments of the invention, wherein the patient has an elevated risk of a cardiovascular adverse event from administration of an antipsychotic agent.

### Embodiment Z

A method of Embodiment [T] above, or according to other embodiments of the invention, wherein the method results in a cardiovascular adverse event in less than or equal to 5% of patients during the 29-day treatment period.

### Embodiment AA

A method of Embodiment [U] above, or according to other embodiments of the invention, wherein the method results in a cardiovascular adverse event in less than or equal to 6% of patients during the 26-week treatment period.

### Embodiment AB

A method of any one of Embodiments [A] to [V] above, or according to other embodiments of the invention, wherein the method results in a cardiovascular adverse event in a percentage of patients that is about the same as or similar to placebo.

### Embodiment AC

A method of any one of Embodiments [W] to [AB] above, or according to other embodiments of the invention, wherein a cardiovascular adverse event is characterized as atrial tachycardia, bradycardia, cardiovascular insufficiency, palpitations, postural tachycardia syndrome, increased blood pressure, hypertension, hypotension, hot flush, QT prolongation, orthostatic hypotension, or orthostatic tachycardia.

### Embodiment AD

A method of any one of Embodiments [A] to [V] above, or according to other embodiments of the invention, wherein the method minimizes extrapyramidal adverse events.

### Embodiment AE

A method of any one of Embodiments [A] to [V] or [AD] above, or according to other embodiments of the invention, wherein the method results in an extrapyramidal adverse event in less than or equal to 5% of patients.

### Embodiment AF

A method of any one of Embodiments [A] to [V] or [AD] above, or according to other embodiments of the invention, wherein the patient has an elevated risk of an extrapyramidal adverse event from administration of an antipsychotic agent.

### Embodiment AG

A method of any one of Embodiments [AD] to [AF] above, or according to other embodiments of the invention, wherein an extrapyramidal adverse event is characterized as akathisia, restlessness, joint stiffness, musculoskeletal stiffness, nuchal rigidity, postural tremor, or tremor.

### Embodiment AH

A method of any one of Embodiments [A] to [V] above, or according to other embodiments of the invention, wherein the method results in an extrapyramidal adverse event in a percentage of patients that is no more than placebo.

### Embodiment AI

A method of any one of Embodiments [A] to [V] above, or according to other embodiments of the invention, wherein the method is substantially devoid of QT prolongation.

### Embodiment AJ

A method of any one of Embodiments [A] to [V] or [AI] above, or according to other embodiments of the invention, wherein the method results in QT prolongation in less than or equal to 5% of patients.

### Embodiment AK

A method of any one of Embodiments [A] to [V] or [AI] above, or according to other embodiments of the invention, wherein the patient has an elevated risk of QT prolongation from administration of an antipsychotic agent.

### Embodiment AL

A method of Embodiment [T] above, or according to other embodiments of the invention, wherein the method is substantially devoid of QT prolongation during the 29-day treatment period.

### Embodiment AM

A method of any one of Embodiments [A] to [V] above, or according to other embodiments of the invention, wherein the method results in QT prolongation in a percentage of patients that is no more than placebo.

### Embodiment AN

A method of any one of Embodiments [AI] to [AM] above, or according to other embodiments of the invention, wherein QT prolongation is characterized as one or both of:
a QTcF interval in the patient of greater than 450 msec at any time point not present at baseline; and
an increase in QTcF interval from baseline of greater than or equal to 30 msec for at least one post-baseline measurement.

### Embodiment AO

A method of any one of Embodiments [A] to [V] above, or according to other embodiments of the invention, wherein the method minimizes hyperprolactinemia in the patient.

### Embodiment AP

A method of any one of Embodiments [A] to [V] above, or according to other embodiments of the invention, wherein the method results in hyperprolactinemia in a percentage of patients that is no more than placebo.

### Embodiment AQ

A method of any one of Embodiments [A] to [V] above, or according to other embodiments of the invention, wherein the method minimizes orthostatic hypotension in the patient.

### Embodiment AR

A method of any one of Embodiments [A] to [V] or [AQ] above, or according to other embodiments of the invention, wherein the method results in orthostatic hypotension in less than or equal to 5% of patients.

### Embodiment AS

A method of any one of Embodiments [A] to [V] or [AQ] above, or according to other embodiments of the invention, wherein the patient has an elevated risk of orthostatic hypotension from administration of an antipsychotic agent.

### Embodiment AT

A method of any one of Embodiments [A] to [V] above, or according to other embodiments of the invention, wherein the method results in orthostatic hypotension in a percentage of patients that is no more than placebo.

### Embodiment AU

A method of any one of Embodiments [A] to [V] above, or according to other embodiments of the invention, wherein the method minimizes orthostatic tachycardia in the patient.

### Embodiment AV

A method of any one of Embodiments [A] to [V] or [AU] above, or according to other embodiments of the invention, wherein the method results in orthostatic tachycardia in less than or equal to 5% of patients.

### Embodiment AW

A method of any one of Embodiments [A] to [V] or [AU] above, or according to other embodiments of the invention, wherein the patient has an elevated risk of orthostatic tachycardia from administration of an antipsychotic agent.

### Embodiment AX

A method of any one of Embodiments [A] to [V] above, or according to other embodiments of the invention, wherein the method results in orthostatic tachycardia in a percentage of patients that is about the same as or similar to placebo.

### Embodiment AY

A method of any one of Embodiments [A] to [AX] above, or according to other embodiments of the invention, wherein the method results in (i) a reduction from baseline in PANSS total score of at least 17.2 or (ii) an effect size in PANSS total score of at least 0.45.

### Embodiment AZ

A method of Embodiment [AY] above, or according to other embodiments of the invention, wherein the result is measured after 29 days of treatment.

### Embodiment BA

A method of any one of Embodiments [AX] or [AY] above, or according to other embodiments of the invention, wherein the method results in a reduction from baseline in PANSS total score of at least about 30 after 30 weeks of treatment.

### Embodiment BB

A method of any one of Embodiments [A] to [BA] above, or according to other embodiments of the invention, wherein the method results in (i) a reduction from baseline in PANSS positive subscale score of at least 5.5 or (ii) an effect size in PANSS positive subscale score of at least 0.32.

### Embodiment BC

A method of Embodiment [BB] above, or according to other embodiments of the invention, wherein the result is measured after 29 days of treatment.

### Embodiment BD

A method of any one of Embodiments [BB] or [BC] above, or according to other embodiments of the invention, wherein the method results in a reduction from baseline in PANSS positive subscale score of at least about 10 after 30 weeks of treatment.

### Embodiment BE

A method of any one of Embodiments [A] to [BD] above, or according to other embodiments of the invention, wherein the method results in (i) a reduction from baseline in PANSS negative subscale score of at least 3.1 or (ii) an effect size in PANSS negative subscale score of at least 0.37.

### Embodiment BF

A method of Embodiment [BE] above, or according to other embodiments of the invention, wherein the result is measured after 29 days of treatment.

### Embodiment BG

A method of any one of Embodiments [BE] or [BF] above, or according to other embodiments of the invention, wherein the method results in a reduction from baseline in PANSS negative subscale score of at least about 5 after 30 weeks of treatment.

### Embodiment BH

A method of any one of Embodiments [A] to [BG] above, or according to other embodiments of the invention, wherein the method results in (i) a reduction from baseline in PANSS general psychopathology subscale score of at least 9 or (ii) an effect size in PANSS general psychopathology subscale score of at least 0.51.

### Embodiment BI

A method of Embodiment [BH] above, or according to other embodiments of the invention, wherein the result is measured after 29 days of treatment.

### Embodiment BJ

A method of any one of Embodiments [BH] or [BI] above, or according to other embodiments of the invention, wherein the method results in a reduction from baseline in PANSS general psychopathology subscale score of at least about 15 after 30 weeks of treatment.

### Embodiment BK

A method of any one of Embodiments [A] to [BJ] above, or according to other embodiments of the invention, wherein the method results in (i) a reduction from baseline in CGI-S score of at least 1 or (ii) an effect size in CGI-S score of at least 0.52.

### Embodiment BL

A method of Embodiment [BK] above, or according to other embodiments of the invention, wherein the result is measured after 29 days of treatment.

### Embodiment BM

A method of any one of Embodiments [BK] or [BL] above, or according to other embodiments of the invention, wherein the method results in a reduction from baseline in CGI-S score of at least about 1.5 after 30 weeks of treatment.

### Embodiment BN

A method of any one of Embodiments [A] to [BM] above, or according to other embodiments of the invention, wherein the method results in (i) a reduction from baseline in BNSS total score of at least 7.1 or (ii) an effect size in BNSS total score of at least 0.48.

### Embodiment BO

A method of Embodiment [BN] above, or according to other embodiments of the invention, wherein the result is measured after 29 days of treatment.

### Embodiment BP

A method of any one of Embodiments [BN] or [BO] above, or according to other embodiments of the invention, wherein the method results in a reduction from baseline in BNSS total score of at least about 10 after 30 weeks of treatment.

### Embodiment BQ

A method of any one of Embodiments [A] to [BP] above, or according to other embodiments of the invention, wherein the method results in (i) a reduction from baseline in MADRS total score of at least 3.3 or (ii) an effect size in MADRS total score of at least 0.32.

### Embodiment BR

A method of Embodiment [BQ] above, or according to other embodiments of the invention, wherein the result is measured after 29 days of treatment.

### Embodiment BS

A method of any one of Embodiments [BQ] or [BR] above, or according to other embodiments of the invention, wherein the method results in a reduction from baseline in MADRS total score of at least about 5 after 30 weeks of treatment.

### Embodiment BT

A method of any one of Embodiments [A] to [V] above, or according to other embodiments of the invention, comprising treating a symptom of insomnia, anxiety, or headache in the patient.

### Embodiment BU

A method of any one of Embodiments [A] to [V] above, or according to other embodiments of the invention, wherein the method minimizes insomnia, anxiety, headache or any combination thereof in the patient.

### Embodiment BV

A method of any one of Embodiments [BT] or [BU] above, or according to other embodiments of the invention, wherein the risk of insomnia, anxiety, headache, or any combination thereof in the patient is less than placebo.

### Embodiment BW

A method of any one of Embodiments [A] to [BV] above, or according to other embodiments of the invention, wherein Compound 1, or a pharmaceutically acceptable salt thereof, is administered orally and daily at a first dose for 1 to 3 days, followed by administering to the patient Compound 1, or a pharmaceutically acceptable salt thereof, daily at a therapeutic dose, wherein the first dose is less than the therapeutic dose, wherein the neurological or psychiatric disease or disorder is schizophrenia.

### Embodiment BX

A method of any one of Embodiments [A] to [BW] above, or according to other embodiments of the invention, wherein Compound 1, or a pharmaceutically acceptable salt thereof, is administered daily at the first dose on days 1-3, and Compound 1, or a pharmaceutically acceptable salt thereof, is administered daily at the therapeutic dose on days 4-29.

### Embodiment BY

A method of any one of Embodiments [BW] or [BX] above, or according to other embodiments of the invention, wherein the first dose is 50 mg and the therapeutic dose is 75 mg.

### Embodiment BZ

A method of treating schizophrenia in a patient, comprising:
orally administering or having administered to the patient 75 mg daily of Compound 1

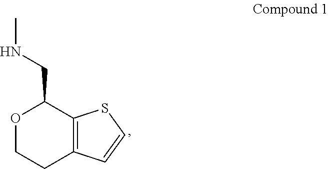

or a pharmaceutically acceptable salt thereof, during a treatment period;
determining or having determined if the patient has experienced an adverse event during the treatment period; and
reducing or having reduced administration to 50 mg daily of Compound 1, or a pharmaceutically acceptable salt thereof, if the patient experiences an adverse event during the treatment period.

### Embodiment CA

A method of treating a symptom of insomnia, anxiety, or headache, in a patient having schizophrenia, comprising administering to the patient a therapeutically effective amount of Compound 1

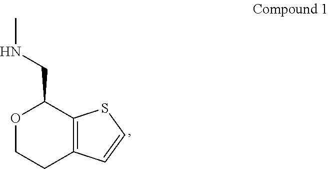

or a pharmaceutically acceptable salt thereof.

### Embodiment CB

A method of any one of Embodiments [A] to [CA] above, or according to other embodiments of the invention, wherein Compound 1, or a pharmaceutically acceptable salt thereof, is Compound 1 hydrochloride of crystalline Form A.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

We claim:

1. (canceled)

2. A method of treating a neurological or psychiatric disease or disorder, in a patient in need thereof, without causing a clinically significant risk of adverse events, comprising administering to the patient a therapeutically effective amount of Compound 1 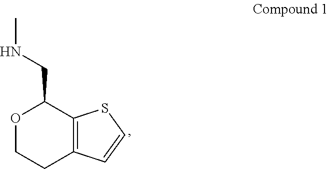
or a pharmaceutically acceptable salt thereof.

3. A method of treating a patient having a neurological or psychiatric disease or disorder without causing a clinically significant risk of adverse events, comprising administering to the patient a therapeutically effective amount of Compound 1 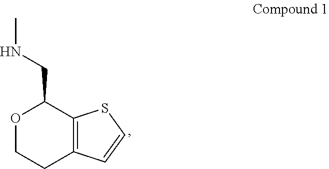
or a pharmaceutically acceptable salt thereof.

4. (canceled)

5. (canceled)

6. A method of treating a neurological or psychiatric disease or disorder in a patient, comprising administering to the patient a therapeutically effective amount of Compound 1 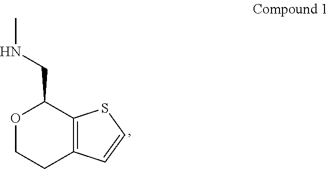
or a pharmaceutically acceptable salt thereof, wherein the method minimizes adverse events associated with antipsychotic agents with affinity to dopamine D2 in the patient.

7. A method of treating a neurological or psychiatric disease or disorder in a patient, comprising administering to the patient a therapeutically effective amount of an antipsychotic agent with no direct affinity to dopamine D2 receptors, wherein the method is substantially devoid of adverse events in the patient, wherein the adverse events are associated with antipsychotic agents with affinity to dopamine D2.

8. The method of claim 7, wherein the antipsychotic agent with no direct affinity to dopamine D2 receptors is Compound 1 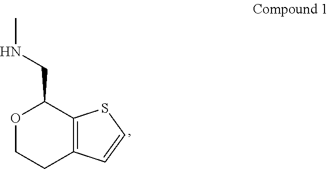
or a pharmaceutically acceptable salt thereof.

9. A method of minimizing adverse events in a patient in need of treatment for a neurological or psychiatric disease or disorder, the method comprising administering to the patient a therapeutically effective amount of an antipsychotic agent with no direct affinity to dopamine D2 receptors, wherein the antipsychotic agent is Compound 1 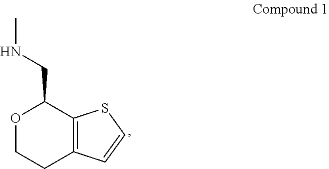
or a pharmaceutically acceptable salt thereof, and wherein the method minimizes adverse events associated with antipsychotic agents with affinity to dopamine D2 receptors.

10. The method of claim 2, wherein the neurological or psychiatric disease or disorder is schizophrenia.

11. The method of claim 2, wherein the neurological or psychiatric disease or disorder is schizophrenia spectrum disorder, schizophrenia negative symptoms, attenuated psychosis syndrome, prodromal schizophrenia, delusional disorder, psychosis, psychotic disorder, delirium, Tourette's syndrome, post-traumatic stress disorder, behavior disorder, affective disorder, depression, bipolar disorder, major depressive disorder, dysthymia, manic disorder, seasonal affective disorder, obsessive-compulsive disorder, narcolepsy, REM behavior disorder, substance abuse or dependency, Lesch-Nyhan disease, Wilson's disease, autism, Alzheimer's disease agitation and psychosis, or Huntington's chorea.

12. The method of claim 2, wherein the neurological or psychiatric disease or disorder is selected from schizophrenia, attenuated psychosis syndrome, prodromal schizophrenia, schizoid personality disorder, and schizotypal personality disorder.

13. The method of claim 11, wherein said psychosis is selected from organic psychosis, drug-induced psychosis, Parkinson's disease psychosis, and excitative psychosis.

14. The method of claim 2, wherein Compound 1, or a pharmaceutically acceptable salt thereof, comprises an HCl salt of Compound 1.

15. The method of claim 2, wherein a risk of adverse events in the patient is about the same as or similar to placebo.

16. The method of claim 2, wherein the method minimizes cardiovascular adverse events.

17. The method of claim 2, wherein the method results in a cardiovascular adverse event in a percentage of patients that is about the same as or similar to placebo

18. The method of claim 16, wherein a cardiovascular adverse event is characterized as atrial tachycardia, bradycardia, cardiovascular insufficiency, palpitations, postural tachycardia syndrome, increased blood pressure, hypertension, hypotension, hot flush, QT prolongation, orthostatic hypotension, or orthostatic tachycardia.

19. The method of claim 2, wherein the method minimizes extrapyramidal adverse events.

20. The method of claim 19, wherein an extrapyramidal adverse event is characterized as akathisia, restlessness, joint stiffness, musculoskeletal stiffness, nuchal rigidity, postural tremor, or tremor.

21. The method of claim 2, wherein the method results in an extrapyramidal adverse event in a percentage of patients that is no more than placebo.

22. The method of claim 2, wherein the method is substantially devoid of QT prolongation.

23. The method of claim 2, wherein the method results in QT prolongation in a percentage of patients that is no more than placebo.

24. The method of claim 22, wherein QT prolongation is characterized as one or both of:
a QTcF interval in the patient of greater than 450 msec at any time point not present at baseline; and
an increase in QTcF interval from baseline of greater than or equal to 30 msec for at least one post-baseline measurement.

25. The method of claim 2, wherein the method minimizes hyperprolactinemia in the patient.

26. The method of claim 2, wherein the method results in hyperprolactinemia in a percentage of patients that is no more than placebo.

27. The method of claim 2, wherein the method minimizes orthostatic hypotension in the patient.

28. The method of claim 27, wherein the method results in orthostatic hypotension in less than or equal to 5% of patients.

29. The method of claim 27, wherein the patient has an elevated risk of orthostatic hypotension from administration of an antipsychotic agent.

30. The method of claim 2, wherein the method results in orthostatic hypotension in a percentage of patients that is no more than placebo.

31. The method of claim 2, wherein the method minimizes orthostatic tachycardia in the patient.

32. The method of claim 31, wherein the method results in orthostatic tachycardia in less than or equal to 5% of patients.

33. The method of claim 31, wherein the patient has an elevated risk of orthostatic tachycardia from administration of an antipsychotic agent.

34. The method of claim 2, wherein the method results in orthostatic tachycardia in a percentage of patients that is about the same as or similar to placebo.
